(19) United States
(12) Patent Application Publication
(10) Pub. No.: US 2025/0092138 A1
(43) Pub. Date: Mar. 20, 2025

(54) COMPOSITIONS INCLUDING SOLID FORMS OF POLYPEPTIDES AND RELATED METHODS

(71) Applicants: Massachusetts Institute of Technology, Cambridge, MA (US); Merck Sharp & Dohme Corp, Rahway, NJ (US)

(72) Inventors: Patrick S. Doyle, Sudbury, MA (US); Jeremy Schieferstein, Medford, MA (US); Paul Reichert, Montville, NJ (US); Chakravarthy Narasimhan, Scotch Plains, NJ (US); Amir Erfani-Khaneghahi, Somerville, MA (US)

(73) Assignees: Massachusetts Institute of Technology, Cambridge, MA (US); Merck Sharp & Dohme Corp, Rahway, NJ (US)

(21) Appl. No.: 18/018,660

(22) PCT Filed: Jul. 30, 2021
(86) PCT No.: PCT/US2021/043916

§ 371 Date: Jan. 30, 2023

Related U.S. Application Data
(60) Provisional application No. 63/059,477, filed on Jul. 31, 2020.

(51) Int. Cl. C07K 16/28; A61K 9/06

(52) U.S. Cl. CPC C07K 16/2818; A61K 9/06; C07K 2317/24

(57) ABSTRACT

Compositions including solid forms of polypeptides such as crystalline antibodies, and related methods, are generally described. The compositions may include carriers such as hydrogels that at least partially encapsulate the solid form of the polypeptides (e.g., crystals, amorphous solids). Encapsulation with certain of the materials described may result in compositions containing relatively high loadings of polypeptides while in some instances retaining structural and functional properties of the polypeptides useful for certain types of administration to subjects (e.g., for prophylactic or therapeutic applications). In some instances, compositions having relatively low dynamic viscosities while having relatively high polypeptide loadings are provided.

### RELATED APPLICATION

This application claims the benefit under 35 U.S.C. § 119 (e) of U.S. Provisional Patent Application No. 63/059,477, filed on Jul. 31, 2020, and entitled “Compositions Including Solid Forms of Polypeptides and Related Methods,” which is incorporated herein by reference in its entirety for all purposes.

### TECHNICAL FIELD

Compositions including solid forms of polypeptides such as crystalline antibodies are generally described.

### BACKGROUND

Polypeptides (e.g., proteins) may be administered to subjects for any of a variety of prophylactic and/or therapeutic reasons. Examples of polypeptides that may be suitable for such uses include antibodies, such as monoclonal antibodies. The effectiveness and ease with which a composition comprising polypeptides can be administered may depend on the concentration of the polypeptide as well as the flow properties of the composition.

Accordingly, the development of improved formulations for the administration of polypeptides is desirable.

### SUMMARY

Compositions including solid forms of polypeptides such as crystalline antibodies, and related methods, are generally described. The compositions may include carriers such as hydrogels that at least partially encapsulate the solid form of the polypeptides (e.g., crystals, amorphous solids). Encapsulation with certain of the materials described may result in compositions containing relatively high loadings of polypeptides while in some instances retaining structural and functional properties of polypeptides useful for certain types of administration to subjects (e.g., for prophylactic or therapeutic applications). In some instances, compositions having relatively low dynamic viscosities while having relatively high polypeptide loadings are provided. The subject matter of the present invention involves, in some cases, interrelated products, alternative solutions to a particular problem, and/or a plurality of different uses of one or more systems and/or articles.

In one aspect, compositions are provided. In some embodiments, a composition comprises a hydrogel and a crystal comprising a solid form of a polypeptide at least partially encapsulated by the hydrogel.

In some embodiments, a composition comprises crystals comprising a solid form of a polypeptide present in an amount of greater than or equal to 1 mg/mL and less than or equal to 500 mg/mL, wherein the composition has a dynamic viscosity that is at least 1.1 times lower than that of an aqueous suspension having an equivalent concentration of crystalline polypeptides under otherwise essentially identical conditions.

In some embodiments, a composition comprises crystals comprising a solid form of a polypeptide associated with one or more hydrogels such that less than or equal to 10 wt % of the crystals are aggregated.

In some embodiments, a composition comprises a hydrogel particle and a solid form of a polypeptide at least partially encapsulated by the hydrogel particle.

In some embodiments, the crystalline polypeptides are at least partially encapsulated by a carrier.

In some embodiments, the carrier comprises a hydrogel.

In some embodiments, the crystals comprising the solid form of a polypeptide are present in an amount of greater than or equal to 50 mg/mL and less than or equal to 500 mg/mL, and wherein the composition has a dynamic viscosity that is at least 4 times lower than that of an aqueous suspension having an equivalent concentration of crystalline polypeptides under otherwise essentially identical conditions.

In some embodiments, the hydrogel comprises covalently cross-linked polymer chains, ionically cross-linked polymer chains, and/or thermally cross-linked polymer chains.

In some embodiments, the cross-linked polymer chains are formed from polymer chains having a molecular weight of less than or equal to 75 kDa.

In some embodiments, the ionically cross-linked polymer chains are cross-linked via a metal ion.

In some embodiments, the hydrogel comprises a cross-linked polyalkylene oxide.

In some embodiments, the polyalkylene oxide comprises polyethylene glycol.

In some embodiments, the hydrogel comprises a cross-linked polysaccharide.

In some embodiments, the polysaccharide comprises alginate.

In some embodiments, the polysaccharide comprises at least partially oxidized alginate.

In some embodiments, the polysaccharide comprises agarose.

In some embodiments, the hydrogel comprises polypeptide chains.

In some embodiments, the hydrogel comprises gelatin.

In some embodiments, at least some of the hydrogel is in the form of particles having the shape of sphere, spheroid, or fiber.

In some embodiments, the particles have an average largest cross-sectional dimension of greater than or equal to 100 microns and less than or equal to 300 microns.

In some embodiments, the particles have an average largest cross-sectional dimension of greater than or equal to 10 microns and less than or equal to 100 microns.

In some embodiments, the particles have an average largest cross-sectional dimension of greater than or equal to 1 micron and less than or equal to 30 microns.

In some embodiments, the particles have an average largest cross-sectional dimension of greater than or equal to 1 micron and less than or equal to 10 microns.

In some embodiments, the particles have an average largest cross-sectional dimension of less than or equal to 1 micron.

In some embodiments, the composition comprises crystalline polypeptides in an amount of greater than or equal to 5 wt %.

In some embodiments, upon exposure to a phosphate buffered saline solution, less than or equal to 90% of the polypeptide is released into the liquid 5 hours after the exposure.

In some embodiments, the polypeptide is active for a period of greater than or equal to 24 months at 5° C. following formation of the composition, as measured by an enzyme-linked immunosorbent activity assay.

In some embodiments, no more than 10% of the polypeptide is degraded or aggregated after a period of greater than or equal to 24 months at 5° C. following formation of the composition.

In some embodiments, at least 90% of the polypeptide is folded in its native state after a period of greater than or equal to 24 months at 5° C. following formation of the composition.

In some embodiments, the crystal comprising the solid form of the polypeptide is crystalline for a period of greater than or equal to 24 months at 5° C. days following formation of the composition, as measured by a second order nonlinear imaging of chiral crystals technique.

In some embodiments, the composition has a dynamic viscosity that is at least 50 times lower than that of an aqueous suspension having an equivalent concentration of non-encapsulated non-crystalline polypeptides under otherwise essentially identical conditions.

In some embodiments, the composition has a dynamic viscosity of less than or equal to 0.3 Pas at a temperature of 25° C. and under a shear rate of 100 s⁻¹.

In some embodiments, the polypeptide can serve as a therapeutic polypeptide, as a prophylactic polypeptide, or as both a therapeutic polypeptide and as prophylactic polypeptide.

In some embodiments, the polypeptides are a first therapeutic and/or prophylactic agent, and the composition further comprises a second therapeutic and/or prophylactic agent.

In some embodiments, the polypeptide is an antibody.

In some embodiments, the polypeptide is a monoclonal antibody.

In some embodiments, polypeptide is a monoclonal antibody of any subtype of IgG.

In some embodiments, the polypeptide is an anti-PD-1 antibody.

In some embodiments, the polypeptide is an anti-PD-1 antibody comprising light chain (LC) complementarity determining regions (CDRs) LC-CDR1, LC-CDR2 and LC-CDR3 comprising a sequence of amino acids as set forth in SEQ ID NOs: 1, 2 and 3, respectively, and heavy chain (HC) CDRs HC-CDR1, HC-CDR2 and HC-CDR3 comprising a sequence of amino acids as set forth in SEQ ID NOs: 6, 7 and 8, respectively.

In some embodiments, the polypeptide is an anti-PD-1 antibody comprising a heavy chain variable region comprising a sequence of amino acids as set forth in SEQ ID NO: 9, or a variant of SEQ ID NO: 9 and a light chain variable region comprising a sequence of amino acids as set forth in SEQ ID NO: 4 or a variant of SEQ ID NO: 4.

In some embodiments, the polypeptide is an anti-PD-1 antibody comprising a heavy chain variable region comprising a sequence of amino acids as set forth in SEQ ID NO: 9 and a light chain variable region comprising a sequence of amino acids as set forth in SEQ ID NO:4.

In some embodiments, the anti-PD-1 antibody is a monoclonal antibody comprising a heavy chain comprising a sequence of amino acids as set forth in SEQ ID NO: 10 or a variant of SEQ ID NO: 10 and a light chain comprising a sequence of amino acids as set forth in SEQ ID NO: 5 or a variant of SEQ ID NO: 5.

In some embodiments, the anti-PD-1 antibody is a monoclonal antibody comprising a heavy chain comprising a sequence of amino acids as set forth in SEQ ID NO: 10 and a light chain comprising a sequence of amino acids as set forth in SEQ ID NO: 5.

In some embodiments, the anti-PD-1 antibody is pembrolizumab or a pembrolizumab variant.

In some embodiments, the anti-PD-1 antibody is pembrolizumab.

In some embodiments, the crystal or crystals comprise a solid form of pembrolizumab complexed to caffeine.

In some embodiments, the crystal or crystals comprise a solid form of pembrolizumab or a pembrolizumab variant produced by a method comprising:
(a) mixing:
   (i) an aqueous buffered solution of pembrolizumab or a pembrolizumab variant,
   (ii) polyethylene glycol (PEG), and
   (iii) an additive selected from the group consisting of caffeine, theophylline, 2′deoxyguanosine-5′-monophosphate, a bioactive gibberellin, and a pharmaceutically acceptable salt of said bioactive gibberellin;
      to form a crystallization solution;
(b) incubating the crystallization solution for a period of time sufficient for crystal formation; and
(c) harvesting the crystalline pembrolizumab or pembrolizumab variant from the solution.

In some embodiments, the additive is caffeine.

In some embodiments, the crystal or crystals comprise a solid form of pembrolizumab or a pembrolizumab variant produced by a method comprising exposing a solution comprising pembrolizumab or the pembrolizumab variant to a precipitant solution at a temperature that is at least 25° C. and is no greater than 50° C. for a time sufficient for crystal formation, wherein the precipitant solution has a pH of 4.0 to 5.0 and comprises 1.0 M to 2.5 M ammonium dihydrogen phosphate.

In some embodiments, the precipitant solution comprises (a) 1.5 M to 2.0 M ammonium dihydrogen phosphate and 100 to 120 mM tris-HCl or (b) 1.9 M ammonium dihydrogen phosphate and 0.09 M ammonium hydrogen phosphate.

In some embodiments, at least some of the solid form of the polypeptide is in the form of a crystal.

In some embodiments, at least some of the solid form of the polypeptide is in the form of an amorphous solid.

In another aspect, methods of delivering a polypeptide are provided. In some embodiments, a method comprises administering to a patient a composition comprising a hydrogel and a solid form of the polypeptide, wherein the polypeptide is at least partially encapsulated by the hydrogel.

In some embodiments, administering the composition to the patient comprises injecting the patient with the composition.

In some embodiments, injecting the patient comprises passing the composition through an aperture such that the composition experiences a shear rate of greater than or equal to 4000 s⁻¹.

In some embodiments, the polypeptide can serve as a therapeutic polypeptide, as a prophylactic polypeptide, or as both a therapeutic polypeptide and as prophylactic polypeptide.

In some embodiments, at least some of the solid form of the polypeptide is in the form of a crystal.

In some embodiments, at least some of the solid form of the polypeptide is in the form of an amorphous solid.

In some embodiments, the polypeptide is an antibody.

In some embodiments, the polypeptide is a monoclonal antibody.

In some embodiments, the polypeptide is a monoclonal antibody of any subtype of IgG.

In some embodiments, the polypeptide is an anti-PD-1 antibody.

In some embodiments, the polypeptide is an anti-PD-1 antibody comprising light chain (LC) complementarity determining regions (CDRs) LC-CDR1, LC-CDR2 and LC-CDR3 comprising a sequence of amino acids as set forth in SEQ ID NOs: 1, 2 and 3, respectively, and heavy chain (HC) CDRs HC-CDR1, HC-CDR2 and HC-CDR3 comprising a sequence of amino acids as set forth in SEQ ID NOs: 6, 7 and 8, respectively.

In some embodiments, the polypeptide is an anti-PD-1 antibody comprising a heavy chain variable region comprising a sequence of amino acids as set forth in SEQ ID NO: 9, or a variant of SEQ ID NO: 9 and a light chain variable region comprising a sequence of amino acids as set forth in SEQ ID NO: 4 or a variant of SEQ ID NO: 4.

In some embodiments, the polypeptide is an anti-PD-1 antibody comprising a heavy chain variable region comprising a sequence of amino acids as set forth in SEQ ID NO: 9 and a light chain variable region comprising a sequence of amino acids as set forth in SEQ ID NO:4.

In some embodiments, the anti-PD-1 antibody is a monoclonal antibody comprising a heavy chain comprising a sequence of amino acids as set forth in SEQ ID NO: 10 or a variant of SEQ ID NO: 10 and a light chain comprising a sequence of amino acids as set forth in SEQ ID NO: 5 or a variant of SEQ ID NO: 5.

In some embodiments, the anti-PD-1 antibody is a monoclonal antibody comprising a heavy chain comprising a sequence of amino acids as set forth in SEQ ID NO: 10 and a light chain comprising a sequence of amino acids as set forth in SEQ ID NO: 5.

In some embodiments, the anti-PD-1 antibody is pembrolizumab or a pembrolizumab variant.

In some embodiments, the anti-PD-1 antibody is pembrolizumab.

In some embodiments, the crystal or crystals comprise a solid form of pembrolizumab complexed to caffeine.

In some embodiments, the crystal or crystals comprise a solid form of pembrolizumab or a pembrolizumab variant produced by a method comprising:
(a) mixing:
   (i) an aqueous buffered solution of pembrolizumab or a pembrolizumab variant,
   (ii) polyethylene glycol (PEG), and
   (iii) an additive selected from the group consisting of caffeine, theophylline, 2′deoxyguanosine-5′-monophosphate, a bioactive gibberellin, and a pharmaceutically acceptable salt of said bioactive gibberellin;
      to form a crystallization solution;
(b) incubating the crystallization solution for a period of time sufficient for crystal formation; and
(c) harvesting the crystalline pembrolizumab or pembrolizumab variant from the solution.

In some embodiments, the additive is caffeine.

In some embodiments, the crystal or crystals comprise a solid form of pembrolizumab or a pembrolizumab variant produced by a method comprising exposing a solution comprising pembrolizumab or the pembrolizumab variant to a precipitant solution at a temperature that is at least 25° C. and is no greater than 50° C. for a time sufficient for crystal formation, wherein the precipitant solution has a pH of 4.0 to 5.0 and comprises 1.0 M to 2.5 M ammonium dihydrogen phosphate.

In some embodiments, the precipitant solution comprises (a) 1.5 M to 2.0 M ammonium dihydrogen phosphate and 100 to 120 mM tris-HCl or (b) 1.9 M ammonium dihydrogen phosphate and 0.09 M ammonium hydrogen phosphate.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows a schematic illustration of an exemplary composition comprising a solid form of a polypeptide, according to some embodiments;

FIG. 2A shows a schematic illustration of an exemplary composition comprising a hydrogel and a solid form of a polypeptide at least partially encapsulated by the hydrogel, according to certain embodiments;

FIG. 2B shows a schematic illustration of an exemplary method of making a composition comprising a hydrogel and a solid form of a polypeptide at least partially encapsulated by the hydrogel, according to certain embodiments;

FIGS. 3A-3B shows schematic diagrams of an exemplary formulation strategy for hydrogel/crystal microspheres, according to certain embodiments;

FIGS. 4A-4B are images of mAb2 crystal suspensions, according to certain embodiments;

FIG. 4C shows a plot of the size distribution of mAb2 crystals, according to certain embodiments;

FIGS. 5A-5E show an exemplary microfluidic method for production of hydrogel particles (FIG. 5A-5B) and characteristic microspheres and associated plots of size distribution (FIGS. 5C-5E), according to certain embodiments;

FIGS. 6A-6F are microscopic images of hydrogel microsphere samples loaded with 0 mg mL⁻¹ (FIG. 6A), 100 mg mL⁻¹ (FIG. 6B), or 300 mg mL⁻¹ (FIG. 6C) of mAb2 crystals, according to certain embodiments;

FIG. 7 shows an image of a hydrogel containing 5 mg/mL mAb2 crystals under high magnification, according to certain embodiments;

FIGS. 8A-8C show plots and analysis of thermogravimetric data of mAb2 antibodies in hydrogels using differential thermograms, according to certain embodiments;

FIGS. 9A-9B show plots and thermogravimetric analysis of hydrogels loaded with mAb2 crystals, according to certain embodiments;

FIGS. 10A-10B are images and associated data plots demonstrating the in vitro release from hydrogel microspheres loaded with mAb2 crystals, according to certain embodiments;

FIGS. 11A-11D are plots of flow curves for mAb2 samples in the form of a suspension of non-encapsulated crystals (squares), hydrogel microspheres with encapsulated crystals (triangles), and a comparable volume fraction of hydrogel microsphere without mAb2 (circles), according to certain embodiments;

FIG. 12A shows a schematic procedure for rheometry measurements of compositions, according to certain embodiments;

FIG. 12B shows a plot of flow curves of concentrated mAb2 solutions, according to certain embodiments;

FIG. 12C shows a plot of flow curves of hydrogel microsphere suspensions without mAb2 present, according to certain embodiments;

FIG. 12D shows a plot of flows curves of a hydrogel microsphere suspension under rough and smooth conditions to test for slip, according to certain embodiments;

FIGS. 13A-13D show the chemical precursors and schematic of a hydrogel formation, according to certain embodiments.

FIGS. 14A-14B show results from of gelation time measurements for a hydrogel as a function of pH and base added, according to certain embodiments.

FIGS. 15A-15C show results from the production of vinylsulfone/thiol crosslinked hydrogel microspheres, according to certain embodiments;

FIGS. 16A-16B show images demonstrating the qualitative dissolution of mAb2 crystals into phosphate-buffered saline (PBS) from vinylsulfone/thiol crosslinked hydrogel microspheres, according to certain embodiments;

FIG. 17 is a graph depicting the quantitative release of mAb2 crystals from the vinylsulfone/thiol crosslinked hydrogels, measured with the Bradford method, according to certain embodiments;

FIG. 18 is a plot showing data from resulting particle diameters after the production of alginate hydrogel particles containing mAb2 crystals, according to certain embodiments;

FIGS. 19A-19B are images of mAb2 crystal-loaded alginate microparticles, according to certain embodiments;

FIGS. 20A-20C are bright-field microscopy images showing the qualitative dissolution of mAb2 crystals from the alginate hydrogel microspheres, according to certain embodiments;

FIG. 21 is a plot showing data of the release of mAb2 crystals from the alginate hydrogels over 120 minutes, according to certain embodiments;

FIGS. 22A-22C show bright field (FIGS. 22A and 22C) and crossed-polarized (FIG. 22B) microscopy images of mAb2 crystal-laden PEG-VS hydrogel particles having diameters of 10-30 microns, according to certain embodiments;

FIGS. 23A-23B show bright field (FIG. 23A) and crossed-polarized (FIG. 23B) microscopy images of mAb2 crystal-laden PEG-VS hydrogel particles having diameters of 1-5 microns, according to certain embodiments;

FIGS. 24A-24B show bright field (FIG. 24A) and crossed-polarized (FIG. 24B) microscopy images of mAb2 crystal-laden PEG-VS hydrogel particles having diameters of less than 1 micron, according to certain embodiments;

FIG. 25 shows a schematic process diagram of the experimental setup for a centrifugal extrusion method, according to certain embodiments;

FIG. 26A shows a plot of aspect ratios of hydrogel particles as a function of collection distance, and associated microscopy images, according to certain embodiments;

FIG. 26B shows a plot of aspect ratios of hydrogel particles as a function of centrifugation speed, and associated microscopy images, according to certain embodiments;

FIG. 27 shows data for antibody crystal dissolution versus concentration of calcium chloride (CaCl₂)) in an aqueous solution that receives the droplets (top), and associated images of the resulting particles with increasing CaCl₂) concentration (bottom), according to certain embodiments;

FIG. 28A shows microscopy images of hydrogels of 1% VLVG and VLVM in the absence of mAb2 crystals (top) and hydrogels of 1% VLVG with 250 mg/mL of encapsulated mAb2 crystals (bottom), according to certain embodiments;

FIG. 28B shows microscopy images of hydrogels of 1% MVG and MVM in the absence of mAb2 crystals (top) and hydrogels of 1% MVG with 250 mg/mL of encapsulated mAb2 crystals (bottom), according to certain embodiments;

FIG. 29A shows a microscopy image of a precursor composition comprising amorphous mAb2, according to certain embodiments;

FIG. 29B shows a microscopy image of an amorphous mAb2-laden alginate hydrogel particle composition, according to certain embodiments;

FIGS. 30A-30B show bright field (FIG. 30A) and crossed-polarized (FIG. 30B) microscopy images of mAb2 crystal-laden alginate hydrogel fiber particles, according to certain embodiments;

FIGS. 31A-31C show microscopy images of mAb2 crystal-laden gelatin hydrogel particles formed via thermal gelling and batch emulsification polymerization techniques, according to certain embodiments;

FIG. 32A shows microscopy images of hydrogel particles formed with partially oxidized alginates, according to certain embodiments;

FIG. 32B shows microscopy images of mAb2 crystal-laden hydrogel particles formed with partially oxidized alginates, according to certain embodiments;

FIG. 33A shows plots of cell viability of NIH Raw 264.7 cells exposed to alginate hydrogel particles at varying concentrations, according to certain embodiments;

FIG. 33B shows plots of the amount of the cytokine TNF α (Tumor Necrosis Factor alpha) secretion from NIH Raw 264.7 cells exposed to alginate hydrogel particles at varying concentrations, according to certain embodiments; and

FIG. 34 shows bright field (VIS; left column), ultraviolet two-photon excited fluorescence (UV-TPEF; middle column), and second harmonic generation (SGH; right) microscopy images of a free suspension of amorphous mAb2 (top row, labeled as “AS” for amorphous suspension) and amorphous mAb2 solid-laden alginate hydrogel particles (bottom row, labeled as “Encap” for encapsulated), according to certain embodiments . . .

### DETAILED DESCRIPTION

Compositions including solid forms of polypeptides such as crystalline antibodies, and related methods, are generally described. The compositions may include carriers such as hydrogels that at least partially encapsulate the solid form of the polypeptides (e.g., crystals, amorphous solids). Encapsulation with certain of the materials described may result in compositions containing relatively high loadings of polypeptides while in some instances retaining structural and functional properties of polypeptides useful for certain types of administration to patients (e.g., for prophylactic or therapeutic applications). In some instances, compositions having relatively low dynamic viscosities while having relatively high polypeptide loadings are provided.

Polypeptides such as proteins are common prophylactic and therapeutic pharmaceutical agents administered to patients. However, convenient administration of polypeptides to patients poses a number of challenges. For example, subcutaneous administration of polypeptides is an alternative route of administration for antibodies, which are often administered via intravenous infusion every few weeks. Such intravenous infusion requires the aid of a healthcare professional and may take place over several hours. Subcutaneous administration (and other routes of administration) can require a high concentration of polypeptides (e.g., antibodies) (>100 mg mL⁻¹) to meet reasonable volume requirements for injection (<1.5 mL). However, it has been realized in the context of this disclosure that conventional formulations having high concentrations of polypeptides tend to result in self-association of the polypeptides and cluster formation in solution, which can manifest as high viscosity and/or present immunogenicity and bioavailability issues. High concentration polypeptide solutions may also be susceptible to accelerated degradation due to polypeptide aggregation, which may impact the activity, pharmacokinetics, and safety of the polypeptide. The development of suitable formulations (e.g., comprising polypeptides) for which a desirable combination of properties for administration (e.g., subcutaneous injection) is a significant goal toward greater patient convenience, including greater patient compliance and less invasive administration options.

Certain existing approaches to engineering the properties of a composition (e.g., reducing viscosity) include changing buffer conditions, adding thinning excipient, or making minor modifications to the polypeptides themselves. However, these approaches can require laborious optimization that may need to be repeated for different polypeptides. The use of solid forms of polypeptides may impart a formulation with certain flow properties, greater solubility, enhanced stability, and tunable release properties. Solid forms such as the crystalline form of polypeptides (e.g., proteins), while traditionally used for purification and structural characterization, may also be utilized to stabilize high concentration formulations of polypeptides such as antibodies, analogously to approaches used with small molecules. However solid form approaches to delivery of polypeptides are complex due to potential challenges related to retaining polypeptide functional and structural stability across phases and conditions. Further, some suspensions of polypeptide crystals exhibit different viscosities when compared to aqueous polypeptide solutions of equivalent concentrations. Due to difficulties in developing polypeptide crystal formulations (e.g. finding safe, suitable crystallization and stabilization conditions; scale-up of crystallization batch size), there has been limited commercial success outside of crystalline insulin.

The inventors of the present disclosure have realized that it may be possible to provide compositions comprising solid forms of polypeptides having satisfactory properties (e.g., loading, flow properties, stability, safety). One approach realized herein is the use of carrier materials (e.g., a solid carrier) for associating (e.g., encapsulating) solid forms of polypeptides (e.g., antibodies) and, in some instances, affording advantageous flow and/or stability properties. For example, it has been realized that hydrogel materials may be a suitable carrier for solid forms of polypeptides, in some cases resulting in compositions capable of relatively high loadings, relatively low viscosities, and good stability (e.g., for prophylactic or therapeutic applications).

In one aspect, compositions comprising polypeptides are described. The composition may, for example, comprise a solid form of a polypeptide in the form of a crystal (e.g., crystalline polypeptides) or in the form of an amorphous solid, as described in more detail below. The compositions described herein may, in some instances, be suitable for administration to a patient (e.g., a mammal such as a human).

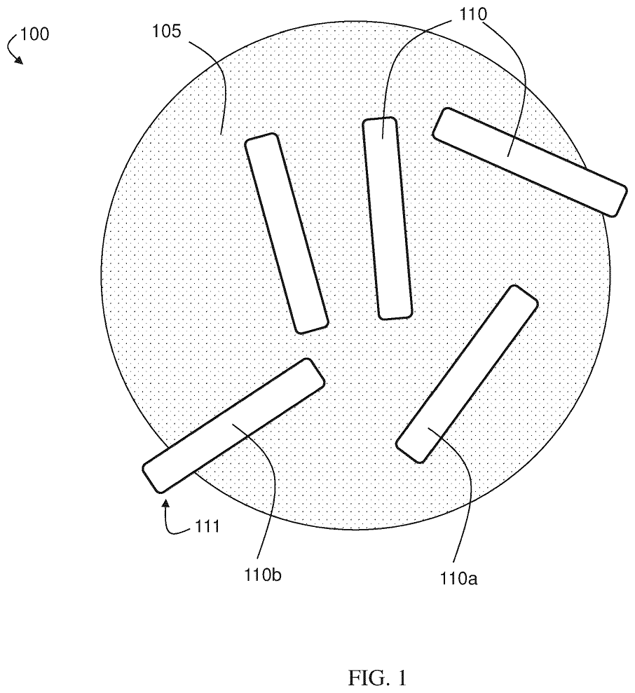

In some, but not necessarily all, embodiments, the composition comprises a carrier associating (e.g., at least partially encapsulating) the solid form of the polypeptides (e.g., the crystalline or amorphous polypeptides). FIG. 1 shows a schematic illustration of composition 100 comprising carrier 105 and crystals 110 each comprising a solid form of a polypeptide (e.g., an antibody), according to certain embodiments. In some embodiments, crystals comprising a solid form of a polypeptide are associated with one or more carriers (e.g., hydrogels) such that less than or equal to 10 wt % (e.g., less than or equal to 5 wt %, less than or equal to 2 wt %, less than or equal to 1 wt %, or none) of the crystals are aggregated, as determined by size-exclusion chromatography-high performance liquid chromatography (SEC-HPLC). For example, the composition may comprise a solid form of a polypeptide associated with one or more hydrogels such that less than or equal to 10 wt % (e.g., less than or equal to 5 wt %, less than or equal to 2 wt %, less than or equal to 1 wt %, or none) of the crystals are aggregated after a period of greater than or equal to 1 hour, greater than or equal to 1 day, greater than or equal to 1 week, greater than or equal to 1 month, greater than or equal to 2 months, greater than or equal to 3 months, and/or up to 6 months, up to 12 months, up to 24 months or greater at 5° C. following formation of the composition.

Association between a solid form of a polypeptide (e.g., crystalline polypeptides) and a carrier can be in the form of specific or non-specific interactions, such as physical interactions (e.g., mechanical confinement, physisorption, etc.) or chemical interactions (e.g., electrostatic interactions, van der Waals interactions, hydrophobic interactions, etc.).

One type of the association is encapsulation. In FIG. 1, crystals 110 are at least partially encapsulated by carrier 105. A molecule or particle is at least partially encapsulated by a carrier (e.g., a hydrogel as described below) if at least a portion of the molecule or particle is confined within the carrier (e.g., for example within a pore of the carrier). It should be understood that a portion (e.g., up to 10 volume percent (vol %), up to 25 vol %, up to 50 vol %, up to 75 vol %, or up to 90 vol %) of a volume occupied molecule or particle, such as a crystal, may protrude from the volume occupied by a carrier (e.g., hydrogel) and still be at least partially encapsulated by the carrier. For example, referring back to FIG. 1, while crystal 110a is completely confined within carrier 105 and crystal 110b includes portion 111 protruding from carrier 105, both crystal 110a and crystal 110b are considered at least partially encapsulated by carrier 105. In some embodiments, at least 10%, at least 25%, at least 50%, at least 75%, at least 90%, at least 95%, at least 99%, or all of the volume of the crystals are confined within the carrier when the crystals are at least partially (e.g., entirely) encapsulated by the carrier. As an illustrative calculation, if a composition has a total volume of crystals comprising a solid form of a polypeptide of 0.1 cm³, and a volume totaling 0.025 cm³ of those crystals is confined within the carrier (e.g., as determined by a suitable imaging technique), then at least 25% of the volume of the crystals are confined within the carrier because 0.025 cm³/0.01 cm³×100%=25%. In some embodiments, at least 10%, at least 25%, at least 50%, at least 75%, at least 90%, at least 95%, at least 99%, or all of the weight of the crystals are confined within the carrier. As an illustrative calculation, if a composition has a total weight of crystals comprising a solid form of a polypeptide of 10 mg, and a weight totaling 2.5 mg of those crystals is confined within the carrier (e.g., as determined by dissolving and quantifying crystals not confined within the carrier), then at least 25% of the weight of the crystals are confined within the carrier because 2.5 mg/10 mg×100%=25%.

Encapsulation of a solid form of a polypeptide (e.g., an antibody) may, in some instances, provide for any of a variety of advantages. For example, a carrier may be useful for masking otherwise poor flow properties of the solid form of the polypeptides (e.g., high viscosity), stabilizing the polypeptides, and/or modulating release of the polypeptide (e.g., following administration).

Any of a number of certain types of carriers may be employed in compositions described in this disclosure. A carrier generally refers to a substance supporting or conveying another substance. A suitable carrier can be selected, with the benefit of this disclosure, based on any of a number of criteria, including the size of the molecule/particle to be carried/associated/encapsulated, the solubility of the molecule/particle to be carried/associated/encapsulated (e.g., in a delivery medium), a desired release rate, desired stability, desired biocompatibility, desired pharmacological properties, desired rheological properties, and desired loading/density. The carrier may have a distinct chemical composition than that of a molecule/particle associated with (e.g., at least partially encapsulated by) the carrier. In some embodiments, the carrier comprises a solid material. The solid material may provide a matrix in which the solid form of the polypeptides may reside. The matrix may comprise a network of polymer chains, such as in hydrogels, described in further detail below. The carrier may be a composition that is capable of being suspended in a liquid to form a suspension solution. In some embodiments, the carrier is a particulate or vesicular composition. The carrier may be, for example, a colloidal carrier, which generally refers to a broad class of substances capable of encapsulating molecules or particles within a microparticle or nanoparticle shell. Examples of carriers include, but are not limited to, micelles, liposomes, polymeric particles, hydrogels (as described in more detail below), inorganic particles (e.g., porous particles), dendrimers, microspheres, nanospheres, quantum dots, and combinations thereof. In some embodiments, a composition comprising a carrier at least partially encapsulating polypeptides further comprises a liquid component. For example, a liquid solvent such as an aqueous buffer may be present within the composition.

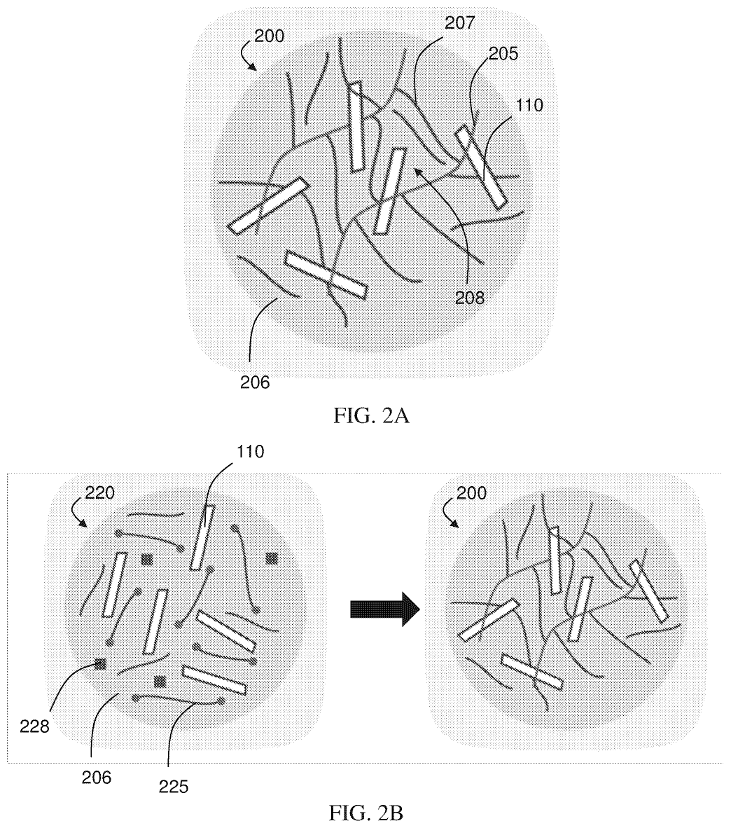

As mentioned above, in some embodiments, the carrier composition comprises a hydrogel. The hydrogel may be a carrier of the composition as described above. FIG. 2A is a schematic illustration of one such embodiment, showing composition 200 comprising a hydrogel at least partially encapsulating crystals 110 comprising a solid form of a polypeptide. A hydrogel generally refers to a three-dimensional network of cross-linked polymer chains that is highly absorbent of water. In some embodiments, the hydrogel can comprise water in an amount of at least 50 weight percent (wt %), at least 60 wt %, at least 75 wt %, at least 90 wt %, or more. Referring again to FIG. 2A, composition 200 comprises a hydrogel comprising a network of polymer chains 205 with cross-links 207 and aqueous solvent 206. Crystals 110 may be present, for example, in pores 208 of the hydrogel of composition 200.

It has been discovered in the context of the present disclosure that a variety of types of hydrogels may be suitable for the compositions and methods relating to polypeptides provided. For example, certain hydrogels formulations may have desirable flow characteristics (e.g., rheological properties such as shear-thinning, thixotropy, viscosity in certain ranges) or structural characteristics (e.g., pore size) for administration of relatively high concentrations of polypeptides (e.g., antibodies). Hydrogels may generally be characterized by the nature of the base polymer of the polymer chains of the hydrogel, or by the nature of the cross-linking.

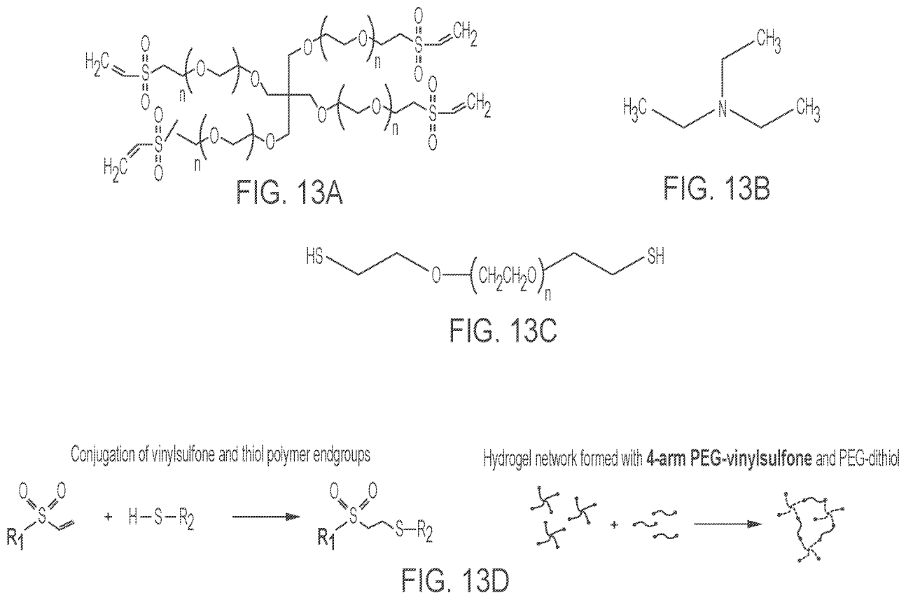

In some embodiments, the hydrogel comprises covalently cross-linked polymer chains. In such embodiments, covalent chemical bonds connect different polymer chains. The covalent bonds may be formed via a variety of chemistries. For example, a photo-initiated chemical reaction may cause a covalent cross-linking. One such example is a radical polymerization, where a reaction mixture is irradiated with electromagnetic radiation (e.g., ultraviolet light) that initiates a cross-linking reaction (e.g., via photoinitiator). Some radical polymerizations involve precursor polymer chains comprising certain functional groups amenable to radical coupling chemistry, such as acrylate or methacrylate groups. Examples of photoinitiators include, but are not limited to, alkylphenones, acetophenones, benzoin ethers, acyl phosphine oxides, and benzophenones. Another example of a suitable covalent cross-linking chemistry is thiol-ene reaction chemistry, where, for example, a polymer chain comprising a thiol functional group covalently bonds with a polymer chain comprising a vinyl sulfone functional group to form a thioether covalent cross-link. FIGS. 13A-13D show exemplary schematics showing one example of hydrogel formation via thiol-ene chemistry. Thiol-ene cross-linking may be accelerated using a catalyst. One example of suitable catalyst is a base, such as an amine (e.g., triethylamine). Another example of a suitable covalent cross-linking chemistry is Michael addition chemistry (e.g., involving vinyl sulfone or maleimide functional groups or the like).

In some embodiments, the hydrogel comprises ionically cross-linked polymer chains. In such embodiments, electrostatic interactions (e.g., via ionic bonds) connect different polymer chains. The ionically cross-linked polymer chains may be cross-linked via a metal ion (e.g., a multivalent metal that non-covalently attracts the different polymer chains). Examples of suitable ions include, but are not limited to calcium ions (Ca²⁺), magnesium ions (Mg²⁺), etc. However, in some embodiments, different polymer chains of the hydrogel may be attracted electrostatically even in the absence of species such as a metal ion. For example, the hydrogel may comprise a first type of polymer chain carrying a positively charged moiety (e.g., an ammonium group) and a second type of polymer chain carrying a negatively charged moiety (e.g., a carboxylate group), and the oppositely charged moieties may for an ionic cross-link.

In some embodiments, the hydrogel comprises thermally cross-linked polymer chains. In such embodiments, a cross-linking may be induced via a temperature change (e.g., cooling or heating). Examples of hydrogels that may undergo thermal cross-linking (e.g., via a temperature method) include, but are not limited to, those comprising thermosensitive polymers such as gelatin and agarose.

The base polymers of the polymer chains may have any of a variety of suitable structures. In some embodiments, the polymer chains of the hydrogel comprise a cross-linked polyalkylene oxide. The repeating unit of the polyalkylene oxide may have any of a variety of a number of carbons, such as from 2 to 18. In some embodiments, the repeating unit of the polyalkylene oxide may have a backbone having any of a variety of a number of carbons, such as from 2 to 10. In some embodiments, the polymer chains of the hydrogel comprise a cross-linked polyethylene glycol (PEG). It should be understood that chemical entities (e.g., polyethylene glycol) described herein encompass optional substitutions/derivatizations. For example, a polymer chain comprising a polyalkylene oxide may further comprise terminal functional groups. As a specific example, polymer chains comprising terminal acrylate terminal groups (e.g., polyethylene glycol diacrylate (PEGDA)) may be used to form a hydrogel comprising cross-linked polymer chains comprising polyethylene glycol. The polymer chains may be branched or unbranched. In some embodiments, the hydrogel comprises a cross-linked polysaccharide. Examples of suitable polysaccharides (which are understood to comprise optional substitutions) include, but are not limited to, alginate, agarose, chitosan, hyaluronic acid, and cellulose. In some embodiments, the hydrogel comprises polypeptide chains. The polypeptide chains may be cross-linked (e.g., covalently, ionically, thermally). In some embodiments, the hydrogel comprises gelatin. Other potentially suitable polymers that may be part of the cross-linked polymer chains of the hydrogel include, but are not limited to, polylactide, poly(glycolic acid), poly(propylene fumarate), polycaprolactone, polyhydroxybutyrate, polyacrylates, poly(vinylpyrrolidone), poly(ethylenimine), and poly(vinylalcohol). Some exemplary hydrogels and conditions for their synthesis are provided in Daly, A. C., Riley, L., Segura, T., & Burdick, J. A. (2019). “Hydrogel microparticles for biomedical applications.” Nature Reviews Materials, 5(1), 20-43, which is incorporated by reference herein in its entirety.

In some, but not necessarily all embodiments, the cross-linked chains of the hydrogel are formed of polymer chains having relatively low molecular weight. It has been observed in the context of this disclosure that relatively low molecular weight polymer chains with relatively low viscosity (e.g., less than or equal to 20 mPa sec), may be used to form hydrogel particles. Such low molecular weight polymer chains may result in hydrogels (e.g., hydrogel particles) having relatively low viscosity (e.g., dynamic viscosity). Use of relatively low-molecular weight and relatively low viscosity polymer chains for forming hydrogel particles may be advantageous in at least some applications (e.g., administering compositions via needles) at least because such hydrogel particles may, in some instances, have enhanced biodegradability. In some embodiments, the cross-linked polymer chains are formed from polymer chains having a molecular weight of less than or equal to 75 kDa, less than or equal to 50 kDa, less than or equal to 25 kDa, or less. In some instances, the cross-linked polymer chains are formed from polymer chains having a molecular weight as low as 20 kDa, as low as 10 kDa, or less. Unless clearly described otherwise, molecular weights of polymers described in this disclosure refer to weight average molecular weights. In some embodiments, the cross-linked polymer chains are formed from polymer chains having a dynamic viscosity of less than or equal to 20 mPa sec, less than or equal to 10 mPa sec. and/or as low as 5 mPa sec or less.

In some, but not necessarily all embodiments, the cross-linked chains of the hydrogel are formed of polysaccharide polymer chains that are at least partially oxidized. For example, the polymer chains may be partially oxidized. It has been observed in the context of this disclosure that at least partial oxidation of polymer chains can, in some instances, enhance biodegradability. In some embodiments, the polysaccharide polymer chains comprise at least partially oxidized alginate. An oxidized polysaccharide may be prepared using any of a variety of suitable oxidizing agents, such as periodate. As a non-limiting example, an alginate polysaccharide can be treated with sodium periodate, resulting in cleavage of the carbon-carbon bond of at least some cis diol groups of uronate residues of the polymer chains (e.g., forming aldehyde groups). In some embodiments, the extent of oxidation of the polysaccharide chains of the at least partially oxidized polymer (e.g., oxidized alginate) is less than or equal to 5 mole percent (mol %), less than or equal to 3 mol %, less than or equal to 1.5 mol %, or less. In some embodiments, the extent of oxidation of the polysaccharide chains of the at least partially oxidized polymer (e.g., oxidized alginate) is greater than or equal to 0.1 mol %, greater than or equal to 0.2 mol %, greater than or equal to 0.5 mol %, greater than or equal to 1 mol %, or greater. The extent of oxidation may be varied by controlling the amount of oxidizing agent (e.g., periodate) exposed to the polymer chains.

In some embodiments, the composition comprises a polypeptide. A polypeptide generally has one or more chains of amino acids linked by peptide (amide) bonds. The amino acids may comprise the standard 20 natural amino acids, or they may include other amino acids (e.g., non-natural and/or non-proteinogenic amino acids such as selenocysteine and pyrrolysine). In some embodiments, the polypeptide has a relatively small number of amino acids. For example, the polypeptide may have as few as 50, as few as 40, as few as 30, as few as 25, as few as 20, as few as 15, as few as 10, or as few as 5 amino acids in its peptide chain. However, in some embodiments, the polypeptide has a relatively large number of amino acids. While in some instances the polypeptide lacks a defined conformation, in other instances the polypeptide has a stable conformation (e.g., for a biological function). In some embodiments the polypeptide is a protein (e.g., a folded protein), which generally has greater than or equal to 50 (e.g., greater than or equal to 60, greater than or equal to 75, greater than or equal to 100, or more) amino acids in its peptide chain and one or more biological functions, such as molecular recognition/affinity-based binding, catalyzing chemical reactions, or providing structural support for a cell. In other embodiments, the polypeptide is a non-protein peptide, such as a peptide hormone (e.g., glucagon or glucagon-like peptide-1). In some embodiments, the polypeptide has greater than or equal to 5, greater than or equal to 10, greater than or equal to 20, greater than or equal to 50, greater than or equal to 100, greater than or equal to 200, greater than or equal to 500, and/or up to 1,000, up to 2,000, up to 5,000, up to 10,000, up to 20,000, or more amino acids in its peptide chain. In certain embodiments, the polypeptide comprises multiple peptide chains. In some embodiments, the multiple peptide chains are not covalently linked. In other embodiments, the multiple peptide chains are covalently linked.

In some embodiments in which the polypeptide comprises a protein, the polypeptide is an antibody. An antibody is an immunoglobulin (Ig) molecule capable of specific binding to an antigen through at least one antigen recognition site, located in the variable region of the immunoglobulin molecule. In some embodiments, the polypeptide is a monoclonal antibody (mAb). Monoclonal antibodies generally refer to antibodies that are made by identical immune cells that are clones of a unique parent cell. Monoclonal antibodies can be therapeutics and/or prophylactics, and some are known for their high specificity and versatility for the treatment of cancer and autoimmune disorders.

As used herein, the term “antibody” encompasses not only intact (i.e., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class/sub-type thereof). Depending on the antibody amino acid sequence of the constant domain of its heavy chains, immunoglobulins can be assigned to different classes. Classes of antibodies may be further divided into subclasses/subtypes (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. The antibodies described herein can be murine, rat, human, or any other origin (including chimeric or humanized antibodies). In some embodiments in which the polypeptide is a monoclonal antibody, the monoclonal antibody may be of any of the abovementioned classes (e.g., IgA, IgD. IgE, IgG, IgM). For example, in some such embodiments, the monoclonal antibody is of any subtype of IgG.

In some embodiments, the polypeptide can serve as a therapeutic polypeptide, as a prophylactic polypeptide, or as both a therapeutic polypeptide and as prophylactic polypeptide, details of which are described further below.

In some embodiments, the polypeptide can serve as a therapeutic polypeptide. That is, in some instances the polypeptide (e.g., protein such as antibody) has at least one indication for which the polypeptide may be used for medical treatment (e.g., post diagnosis). In this context, “treat” or “treatment” generally refers to administering an agent, such as a composition containing an active pharmaceutical ingredient, internally or externally to a subject or patient having one or more disease symptoms, or being suspected of having a disease, for which the agent has therapeutic activity. Typically, the agent is administered in an amount effective to alleviate one or more disease symptoms in the treated subject or population, whether by inducing the regression of or inhibiting, delaying or slowing the progression of such symptom(s) by any clinically measurable degree. The amount of an agent that is effective to alleviate any particular disease symptom may vary according to factors such as the disease state, age, and weight of the patient, and the ability of the composition to elicit a desired response in the subject. Whether a disease symptom has been alleviated can be assessed by any clinical measurement typically used by physicians or other skilled healthcare providers to assess the severity or progression status of that symptom. The terms further include a postponement or development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms generally denote that a beneficial result has been conferred on a vertebrate subject with a disorder, disease or symptom, or with the potential to develop such a disorder, disease or symptom.

In some instances, a therapeutic polypeptide can be used to treat diseases in which the polypeptide is lacking or deficient (e.g., insulin). In some instances, a therapeutic polypeptide can be used to treat diseases by inhibiting or initiating a biological process. For example, a therapeutic antibody (e.g., monoclonal antibody) may treat a disease by inhibiting cell growth rates (e.g., tumor cells) or triggering an immune response. Examples of potential therapeutic polypeptides include, but are not limited to, any therapeutic polypeptide having a known crystallization condition. Exemplary classes of therapeutic polypeptides include antibodies (monoclonal antibodies), fusion proteins, anticoagulants, blood factors, bone morphogenetic proteins, engineered protein scaffolds, enzymes, growth factors, hormones, interferons, interleukins, and thrombolytics. Further examples of therapeutic polypeptides (e.g., proteins) can be found in Dimitrov. D. (2012). “Therapeutic Proteins”; Voynov, V., Caravella, J A, Eds. Methods in Molecular Biology, Humana Press, 1-26, which is incorporated by reference herein in its entirety.

As mentioned above, in some embodiments, the polypeptide is a therapeutic antibody. In some embodiments, the polypeptide is a fragment of a therapeutic antibody (e.g., an antigen binding fragment of a therapeutic antibody). In some embodiments, the polypeptide is a therapeutic monoclonal antibody or an antigen binding fragment thereof. In some embodiments, the polypeptide may be a therapeutic monoclonal antibody. In some embodiments, the therapeutic monoclonal antibody is an anti-PD-1 monoclonal antibody. In some embodiments, the polypeptide is an antigen binding fragment of an anti-PD-1 monoclonal antibody. Examples of suitable anti-PD-1 monoclonal antibodies include, but are not limited to, nivolumab, cemiplimab, pidilizumab (as described in U.S. Pat. No. 7,332,582, which is incorporated herein by reference in its entirety), AMP-514 (MedImmune LLC, Gaithersburg, MD), PDR001 (as described in U.S. Pat. No. 9,683,048, which is incorporated herein by reference in its entirety), BGB-A317 (as described in U.S. Pat. No. 8,735,553, which is incorporated herein by reference in its entirety), MGA012 (MacroGenics, Rockville, MD), sintilimab (Innovent Biologics Co., San Mateo, CA), tislelizumab (Beigene, Beijing, China), camrelizumab (Jangsu Hengrui Medicine, Lianyungan, Jiangsui, China), toripalimab (Junshi Biosciences, Shanghai, China), and prolgolimab (Biocad, St. Petersburg, Russian Federation). Another example of an anti-PD-1 monoclonal antibody is pembrolizumab. In some embodiments, the therapeutic monoclonal antibody is an anti-PD-L1 antibody. In some embodiments, the polypeptide is an antigen binding fragment of an anti-PD-L1 monoclonal antibody. Examples of suitable ani-PD-L1 monoclonal antibodies include, but are not limited to, tatezolizumab, durvalumab, avelumab, BMS-936559, and an antibody comprising the heavy chain and light chain variable regions of SEQ ID NO:20 and SEQ ID NO:21, respectively, of International Patent Publication No. WO2013/019906, which is incorporated herein by reference in its entirety.

In some embodiments, the anti-PD-1 antibody is pembrolizumab. Pembrolizumab (formerly known as MK-3475, SCH 900475 and lambrolizumab) alternatively referred to herein as “pembro,” or “mAb2” is a humanized IgG4 mAb with the structure described in WHO Drug Information, Vol. 27, No. 2, pages 161-162 (2013) and which comprises the heavy and light chain amino acid sequences and CDRs described in Table 3 below. Pembrolizumab has been approved by the U.S. FDA as described in the Prescribing Information for KEYTRUDA™ (Merck & Co., Inc., Whitehouse Station, NJ USA; initial U.S. approval 2014, updated July 2021).

In some embodiments, the anti-PD-1 antibody is a pembrolizumab variant. As used herein, pembrolizumab variant means a monoclonal antibody that comprises heavy chain and light chain sequences that are identical to those in pembrolizumab, except for having three, two or one conservative amino acid substitutions at positions that are located outside of the light chain CDRs and six, five, four, three, two or one conservative amino acid substitutions that are located outside of the heavy chain CDRs. e.g. the variant positions are located in the FR regions or the constant region, and optionally has a deletion of the C-terminal lysine residues of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

In some embodiments, the anti-PD-1 antibody, or antigen binding fragment thereof, comprises: (a) light chain CDRs LC-CDR1, LC-CDR2 and LC-CDR3 comprising a sequence of amino acids as set forth in SEQ ID NOs: 1, 2 and 3, respectively, and heavy chain CDRs HC-CDR1, HC-CDR2 and HC-CDR3 comprising a sequence of amino acids as set forth in SEQ ID NOs: 6, 7 and 8, respectively. In some embodiments, the anti-PD-1 antibody or antigen binding fragment thereof is a human antibody. In other embodiments, the anti-PD-1 antibody or antigen binding fragment thereof is a humanized antibody. In other embodiments, the anti-PD-1 antibody or antigen binding fragment thereof is a chimeric antibody. In specific embodiments, the anti-PD-1 antibody or antigen binding fragment thereof is a monoclonal antibody.

In some embodiments, the PD-1 antibody, or antigen binding fragment thereof, specifically binds to human PD-1 and comprises (a) a heavy chain variable region comprising an amino acid sequence as set forth in SEQ ID NO:9, or a variant thereof, and (b) a light chain variable region comprising an amino acid sequence selected from the group consisting of SEQ ID NO:4 or a variant thereof.

A variant of a heavy chain variable region sequence or full-length heavy chain sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence or full-length light chain sequence is identical to the reference sequence except having up to five conservative amino acid substitutions in the framework region (i.e., outside of the CDRs), and preferably has less than four, three or two conservative amino acid substitution in the framework region.

In some embodiments of the treatment methods, compositions, kits and uses of the present disclosure, the PD-1 antibody or antigen-binding fragment thereof is a monoclonal antibody which specifically binds to human PD-1 and comprises (a) a heavy chain comprising or consisting of a sequence of amino acids as set forth in SEQ ID NO: 10, or a variant thereof; and (b) a light chain comprising or consisting of a sequence of amino acids as set forth in SEQ ID NO:5, or a variant thereof.

In some embodiments of the treatment methods, compositions and uses of the present disclosure, the PD-1 antibody or antigen-binding fragment thereof is a monoclonal antibody which specifically binds to human PD-1 and comprises (a) a heavy chain comprising or consisting of a sequence of amino acids as set forth in SEQ ID NO: 10 and (b) a light chain comprising or consisting of a sequence of amino acids as set forth in SEQ ID NO:5.

In some embodiments, the polypeptide is a prophylactic polypeptide. That is, in some instances the polypeptide (e.g., protein such as antibody) has at least one indication for which the polypeptide may be used for preventing a disease from occurring or reducing the likelihood of the disease from occurring (e.g., by at least 25%, by at least 50%, by at least 75%, by at least 90%, by at least 95%, by at least 99%) within a time period of at least one hour, at least one day, at least one week, at least one month, at least one year, and/or up to 2 years, up to 5 years, or more from administration of the polypeptide. For example, an antibody may be administered to a patient as a passive immunization to a disease (e.g., viral diseases such as influenza). Examples of potential prophylactic polypeptides include, but are not limited to, any prophylactic polypeptide having a known crystallization condition. Exemplary classes of prophylactic polypeptides include antibodies (monoclonal antibodies), fusion proteins, anticoagulants, blood factors, bone morphogenetic proteins, engineered protein scaffolds, enzymes, growth factors, hormones, interferons, interleukins, and thrombolytics. In some embodiments, the polypeptide is a prophylactic antibody (e.g., a prophylactic monoclonal antibody) or a fragment thereof (e.g., an antigen binding fragment thereof).

It has been observed, surprisingly, that despite extensive processing involved in the encapsulation, polypeptides in compositions described in this disclosure can maintain stability (e.g., biological activity stability, chemical stability, structural stability, and/or physical stability). Stability in this context generally refers to the polypeptide essentially retaining its biological activity, chemical stability, structural stability, and/or physical stability upon storage. Various analytical techniques for measuring stability are available in the art and are reviewed in Peptide and Protein Drug Delivery, 247-301. Vincent Lee Ed., Marcel Dekker, Inc., New York, N.Y., Pubs. (1991) and Jones, A. Adv. Drug Delivery Rev. 10:29-90 (1993), for example. Stability can be measured at a selected temperature for a selected time period. In some embodiments, the polypeptide is stable at room temperature (25° C.) or at 30° C., or at 40° C. for at least 1 month and/or stable at about 2-8° C. (e.g., 5° C.) for at least 1 year, for at least 2 years, or more (e.g., up to 3 years, up to 4 years, or longer). In some embodiments, the polypeptide is stable following freezing the composition (to, e.g., −70° C.) and thawing of the composition (a “freeze/thaw cycle”).

In some embodiments, polypeptides in compositions described in this disclosure can maintain stability in terms of biological activity. Such activity may be maintained even upon encapsulation by a carrier (e.g., a hydrogel). The activity of a polypeptide can be assessed based on the level to which it can perform a specific function of the polypeptide (e.g., for an intended purpose such as therapeutic or prophylactic applications). For example, if the polypeptide is an enzyme, the enzyme's activity for catalyzing a reaction can be quantitatively assessed using any known activity assay for that enzyme in the art. As another example, if the polypeptide is an antibody, the affinity of the antibody to an antigen can be quantitatively assessed. Such assays can be performed, for example, using an enzyme activity assay such as an enzyme-linked immunosorbent assay (ELISA) in which substrate turnover is measured (e.g., via the production or consumption of a detectable label such as a colored or fluorescent label). Substrate turnover could be measured directly in instances where the polypeptide is an enzyme, or indirectly via binding to an enzyme, for example when the polypeptide is an antibody. In some embodiments, the polypeptide of the composition is active for a period of greater than or equal to 1 month, greater than or equal to 2 months, greater than or equal to 3 months, greater than or equal to 6 months, greater than or equal to 12 months, greater than or equal to 24 months or greater at 5° C. following formation of the composition, as measured by an enzyme-linked immunosorbent activity assay. The assay would be compared to the activity of the polypeptide prior to formation of the composition under otherwise essentially identical conditions (e.g., temperature, buffer, external agitation such as stirring, etc.). The activity of the polypeptide following composition formation may be within 30%, within 20%, within 10%, within 5%, within 2%, within 1%, or equal to the activity of the polypeptide prior to formation of the composition (or at the time of formation) at the time points described above.

In some embodiments, polypeptides in compositions described in this disclosure can maintain stability in terms of chemical stability. A polypeptide generally maintains its chemical stability in a composition if the chemical stability at a given time is such that the antibody is considered to still retain its biological activity as described above. Chemical stability can be assessed by detecting and quantifying chemically altered forms of the polypeptide. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by ion-exchange chromatography, for example. In some embodiments, no more than 10%, no more than 5%, no more than 2%, no more than 1%, or no more than 0.1% of the polypeptides (e.g., proteins such as antibodies) are degraded as measured by size-exclusion chromatography-high performance liquid chromatography (SEC-HPLC) after a period of greater than or equal to 1 month, greater than or equal to 2 months, greater than or equal to 3 months, greater than or equal to 6 months, greater than or equal to 12 months, greater than or equal to 24 months or greater at 5° C. following formation of the composition. In some embodiments, no more than 10%, no more than 5%, no more than 2%, no more than 1%, or no more than 0.1% of the polypeptides (e.g., proteins such as antibodies) are clipped, as measured by a percentage of low molecular weight species using SEC-HPLC, after a period of greater than or equal to 1 month, greater than or equal to 2 months, greater than or equal to 3 months, greater than or equal to 6 months, greater than or equal to 12 months, greater than or equal to 24 months or greater at 5° C. following formation of the composition.

In some embodiments, polypeptides in compositions described in this disclosure can maintain stability in terms of physical stability. A polypeptide generally maintains its physical stability in a composition if it shows substantially no signs of aggregation, precipitation and/or denaturation upon visual examination of color and/or clarity, or as measured by UV light scattering or by size exclusion chromatography. Physical stability may be measured in terms of extent of polypeptide aggregation. In some embodiments, no more than 10%, no more than 5%, no more than 2%, no more than 1%, or no more than 0.1% of the polypeptides (e.g., proteins such as antibodies) are aggregated, as measured by a percentage of high molecular weight species using SEC-HPLC, after a period of greater than or equal to 1 month, greater than or equal to 2 months, greater than or equal to 3 months, greater than or equal to 6 months, greater than or equal to 12 months, greater than or equal to 24 months or greater at 5° C. following formation of the composition.

In some embodiments, polypeptides having defined native three-dimensional structures (e.g., proteins) in compositions described in this disclosure can maintain stability in terms of structural stability. Various analytical techniques for measuring structural stability in terms of protein folding in in vitro conditions are known in the art, including X-ray crystallography, fluorescence spectroscopy, circular dichroism, and protein nuclear magnetic resonance spectroscopy (NMR). In some embodiments, at least 90%, at least 95%, at least 98%, at least 99%, at least 99.9%, or 100% of the polypeptides (e.g., proteins such as antibodies) are folded in their native structure after a period of greater than or equal to 1 month, greater than or equal to 2 months, greater than or equal to 3 months, greater than or equal to 6 months, greater than or equal to 12 months, greater than or equal to 24 months or greater at 5° C. following formation of the composition.

In some embodiments, the polypeptide is present in the composition (e.g., encapsulated at least partially by a hydrogel) in a solid form. It has been realized in the context of this disclosure that inclusion of a solid form of a polypeptide, as opposed to a non-solid form such as dissolved polypeptide (e.g., in an aqueous buffer), can in some instances allow for relatively high concentrations of polypeptides in the composition. Such high concentrations may be desirable in some applications. Encapsulation of a solid form of a polypeptide in a carrier such as a hydrogel may then provide for potential advantages both in concentration (e.g., vs. dissolved polypeptide) and in flow properties (e.g., rheological properties) (e.g., vs. free solid polypeptide). Examples of solid forms of polypeptides include crystalline forms and amorphous forms.

In some embodiments, the solid form of the polypeptide is a crystal. For example, the composition may comprise a hydrogel at least partially encapsulating a crystal comprising the solid form of a polypeptide. Polypeptide crystals may provide for a relatively stable, concentrated form of the polypeptide, which may allow for a relatively long shelf life while being relatively easy to administer. Referring to back to FIGS. 1 and 2A, a polypeptide may be present in composition 100 (FIG. 1) or 200 (FIG. 2) as a solid as part of crystals 110 (see FIG. 1 or FIG. 2A), according to certain embodiments. A crystal comprising a solid form of a polypeptide generally refers to a solid comprising a lattice with regular repeating units that include individual polypeptide molecules. In some embodiments, the crystal comprising the solid form of the polypeptide is single-crystalline, with the crystal lattice extending unbroken to the edges of the crystal with no grain boundaries. However, in some embodiments, the crystal is polycrystalline, containing multiple sub-domains separate by grain boundaries. The composition comprising a crystal may have at least one, at least 2, at least 3, at least 5, at least 10, at least 50, and/or up to 100, up to 1,000 or more crystals comprising a solid form of the solid polypeptide, depending, for example on the size of the crystals, volume of the composition or hydrogel therein, or desired loading. The polypeptide may be present in the crystal in a relatively high amount (e.g., greater than or equal to 50 wt %, greater than or equal to 75 wt %, greater than or equal 90 wt %, greater than or equal to 99 wt %, or 100 wt % excluding solvent). Most crystals comprising polypeptides (e.g., proteins) are chiral, lacking a plane of symmetry. As such, the presence of a polypeptide in a composition in the form of a crystal may be assessed using any of a variety of suitable techniques, including second order non-linear imaging of chiral crystals (SONICC®) techniques. Second order non-linear imaging of chiral crystals is known in the art and is described, for example, in Kissick, D. J., Wanapun, D., & Simpson, G. J. (2011). “Second-order nonlinear optical imaging of chiral crystals.” Annual Review of Analytical Chemistry, 4, 419-437.

In some embodiments, the solid form of the polypeptide is crystalline pembrolizumab or a crystalline pembrolizumab variant. Non-limiting examples of crystalline pembrolizumab, including methods of making the same, are described in WO 2020/092233, published May 7, 2020 on behalf of Merck Sharp & Dohme Corp and WO 2016/137850, published Sep. 1, 2016 on behalf of Merck Sharp & Dohme Corp, each of which is incorporated herein by reference in its entirety. In some embodiments, the solid form of the polypeptide is crystalline pembrolizumab which comprises pembrolizumab complexed to caffeine. In some embodiments, the composition comprises a hydrogel at least partially encapsulating a crystal comprising the solid form of a pembrolizumab. In some embodiments, the composition comprises a hydrogel that is at least partially encapsulating a crystal comprising a solid form of pembrolizumab complexed to caffeine.

Crystalline pembrolizumab complexed with caffeine (an example of a solid form of pembrolizumab complexed to caffeine) may be prepared as described in the Examples herein or as described in WO 2020/092233. Crystalline pembrolizumab (an example of a solid form of pembrolizumab) may also be prepared as described in WO 2016/137850. In some embodiments, the composition comprises a hydrogel at least partially encapsulating a crystal comprising the solid form of a pembrolizumab which is made by the process described in the examples herein, or in WO 2020/092233 or WO 2016/137850.

In some embodiments, the crystal is a crystal of pembrolizumab which is characterized by unit cell dimensions of a=63.5 to 78.9 Å, b=110.2 to 112.2 Å, c=262.5 to 306 Å, α=90, β=90, γ=90° and a space group of P2₁2₁2₁, as described in WO 2016/137850. In further embodiments, the crystal is capable of diffracting X-rays to a resolution selected from the group consisting of 2.3 Å to 3.5 Å. 2.3 Å to 3.0 Å. 2.3 Å to 2.75 Å. 2.3 Å to 2.5 Å and 2.3 Å. In some embodiments, the crystal of pembrolizumab or the pembrolizumab variant is produced by a method comprising exposing a solution comprising pembrolizumab or the pembrolizumab variant to a precipitant solution at a temperature that is at least 25° C. and is no greater than 50° C. for a time sufficient for crystal formation, wherein the precipitant solutions has a pH of 4.0 to 5.0 and comprises 1.0 M to 2.5 M ammonium dihydrogen phosphate. In further embodiments, the precipitant solution comprises (a) 1.5 M to 2.0 M ammonium dihydrogen phosphate and 100 to 120 mM tris-HCl or (b) 1.9 M ammonium dihydrogen phosphate and 0.09 M ammonium hydrogen phosphate.

In some embodiments, the crystal comprises pembrolizumab complexed with caffeine. In some embodiments, the crystal comprises pembrolizumab complexed with caffeine wherein the crystal is characterized by space group P222₁ a=43.8 Å b=113.9 Å c=175.0 Å, α=β=γ=90°. In some embodiments, the crystal comprises pembrolizumab complexed to caffeine, characterized by solid state NMR 13C spectrum exhibiting peaks at about 182.16, 181.54, 179.99, 109.36, 108.23, 103.58, 76.88 and 76.04 ppm. In further embodiments, the crystalline pembrolizumab further exhibits peaks at about 183.07, 180.55, 110.70, 110.15, 101.49, 99.75, 98.56, 74.97, 74.41, 73.52, 72.69, 13.85, 13.27, 12.26 and 11.13 ppm. In some embodiments, the crystals of pembrolizumab or pembrolizumab variant complexed to caffeine are produced by a method comprising (a) mixing (i) an aqueous buffered solution of pembrolizumab or a pembrolizumab variant, (ii) polyethylene glycol (PEG) and (iii) an additive selected from the group consisting of caffeine, theophylline, 2′deoxyguanosine-5′-monophosphate, a bioactive gibberellin, and a pharmaceutically acceptable salt of said bioactive gibberellin; to form a crystallization solution; (b) incubating the crystallization solution for a period of time sufficient for crystal formation; and (c) harvesting the crystalline pembrolizumab or pembrolizumab variant from the solution. In further embodiments, the additive is caffeine.

It has been surprisingly observed in the context of this disclosure that crystals of polypeptides can be relatively stable in the compositions described here in terms of physical stability and/or chemical stability and/or biological stability. In some embodiments, a crystal comprising a polypeptide (e.g., a monoclonal antibody) can be still be crystalline for a period of greater than or equal to 1 month, greater than or equal to 2 months, greater than or equal to 3 months, greater than or equal to 4 months, greater than or equal to 6 months, greater than or equal to 12 months, or greater than or equal to 24 months at 5° C. following formation of the composition, as measured by a second order nonlinear imaging of chiral crystals technique. Whether a crystal remains crystalline can be assessed, for example, using second order non-linear imaging of chiral crystals (SONICC®) techniques described above. Encapsulation of the crystal in a carrier such as a hydrogel may increase the stability of a crystal comprising a polypeptide by, for example, shield the polypeptide from potentially disruptive forces. Surprisingly, methods of encapsulation, including hydrogel formation described in this disclosure may be performed without substantially disrupting crystallinity of the polypeptides.

In some embodiments, the polypeptide is in the form of an amorphous solid. While FIGS. 1 and 2A depict crystals 110, the polypeptide may be present in composition 100 or composition 200 as an amorphous solid. In some such examples, an amorphous solid comprising the polypeptide may be at least partially encapsulated by a carrier (e.g., a hydrogel). An amorphous solid is generally one lacking a definite lattice or geometric shape. In the context of this disclosure, an amorphous solid may be dry (e.g., a dried powder free of or being associated with a relatively small amount of liquid such as adsorbed moisture), or an amorphous solid may be a wet solid (e.g., associated with a liquid such as a powder suspended in a liquid). A polypeptide in the form of an amorphous solid (e.g., a wet, at least partially-encapsulated amorphous solid) stands in contrast to a dissolved polypeptide, where intermolecular attractions between polypeptides are dominated by interactions with the solvent such that a homogeneous phase mixture results.

The polypeptide solids may be provided in any of a variety of suitable ways. Amorphous solids of polypeptide may be available commercially, and may optionally undergo one or more purification steps. In some embodiments where the crystals comprising a solid form of the polypeptide are used, the polypeptide may be crystallized. The polypeptide (e.g., protein such as an antibody) may be crystallized using suitable techniques. Such techniques include, but are not limited to, batch, microbatch, vapor diffusion, hanging drop, microdialysis, and free interface diffusion. Suitable solution conditions from which the crystals are grown may depend on factors such as polypeptide concentration, choice of buffer, pH, temperature of components, and precipitating agents. Crystals of polypeptides may be recovered (e.g., by centrifugation) and stored in a suitable buffer prior to further processing (e.g., encapsulation in a carrier such as a hydrogel).

In some embodiments in which a composition comprises a hydrogel at least partially encapsulating a solid form of a polypeptide, the hydrogel may be formed from a solution containing the solid form of the polypeptide. For example, crystals comprising the polypeptides may be present (e.g., suspended) in a solution comprising precursor components for the hydrogel. Some such solutions may comprise precursor polymer chains and optionally cross-linking agents, initiators (e.g., photoinitiators, chemical initiators such as bases, etc.), porogens, and buffer molecules. Such a solution may be referred to as a “prepolymer” solution. As one example, in embodiments where crystals of antibodies are encapsulated by a polyethylene glycol-based hydrogel, the crystals may first be suspended in a prepolymer solution comprising polyethylene glycol and polyethylene diacrylates (PEGDA).

In some embodiments, an external stimulus is applied to the prepolymer solution comprising the solid form of the polypeptide (e.g., crystalline polypeptide) to cause cross-linking and hydrogel formation. Such an external stimulus may be any of the exemplary stimuli described above (e.g., electromagnetic radiation, chemical catalyst addition). Referring again to the example of a polyethylene glycol-based hydrogel, ultraviolet light may irradiate the prepolymer and cause subsequent radical-based cross-linking and subsequent hydrogel formation around and about the crystals. FIG. 2B shows an example of a composition formation process, where prepolymer solution 220 comprising polypeptide crystals 110, precursor polymer chains 225 (e.g., PEGDA), and initiator 228 in liquid buffer 206 (e.g., a PEG buffer). Crosslinking of precursor polymer chains 225 (e.g., via ultraviolet light) results in composition 200, according to some embodiments.

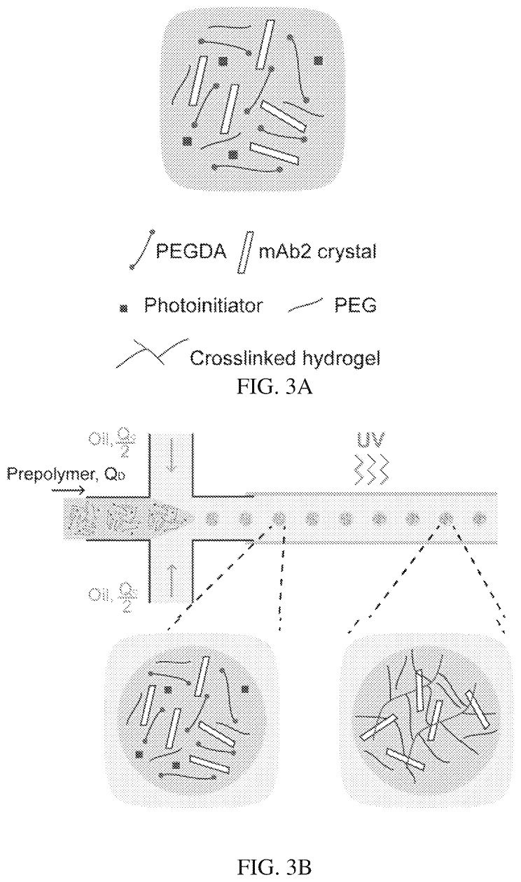

It has been observed that certain forms of carriers (e.g., hydrogels) may be advantageous for certain applications of the compositions. The shape of a hydrogel composition may be tuned using any of a variety of techniques, described in further detail below. One example is a hydrogel microsphere. One way to form suitable hydrogel microspheres is using fluidic (e.g., microfluidic) techniques. For example, a cross-junction technique where a prepolymer phase is flow across the junction in a first direction and an immiscible phase (e.g., an oil phase) is flowed across the junction at a second direction orthogonal to the first direction may be used. Such a flow pattern may result in droplets of prepolymer comprising the solid form of the polypeptide suspended in the immiscible phase. Subsequent initiation of cross-linking and hydrogel formation can occur by flowing the polypeptide-loaded prepolymer droplets past a source of the external stimulus (e.g., a source of electromagnetic radiation). The resulting composition may have dimensions and shapes that dependent on factors such as droplet size and cross-linking conditions, viscosity or surface tension of the prepolymer solution comprising the solid polypeptide, or the geometry of the device used for generating the droplets (e.g., orifice size/diameter). The resulting composition (e.g., comprising hydrogel microspheres comprising solid polypeptide) can be purified by removing the immiscible phase and washing (e.g., with buffer). FIGS. 3A-3B in the Examples below describe one example of such a process.

The solution conditions of the prepolymer during hydrogel formation may influence any of a number of factors, including stability of the solid form of the polypeptide both in terms of activity and in terms of maintaining crystallinity (in embodiments comprising crystalline polypeptides). The solution conditions may also be important for stable formation of the hydrogel. For example, the pH of the prepolymer solution may affect the stability of the polypeptide and/or the safety of the polypeptide (e.g., for therapeutic and/or prophylactic applications), as well as the stability of the hydrogel. It has been discovered in the context of this disclosure that certain pH ranges are suitable for stable polypeptide handling and hydrogel formation. In some embodiments, the pH of the prepolymer solution is greater than or equal to 4, greater than or equal to 5, greater than or equal to 6, greater than or equal to 7, and/or up to 8, up to 9, or up to 10. In some embodiments, the pH of the prepolymer solution is from 5 to 8.

The compositions described herein (e.g., comprising carriers such as hydrogels at least partially encapsulating solid forms of polypeptides such as antibodies) may come in any of a variety of forms. For example, in some embodiments where a composition comprises a hydrogel, the hydrogel is in the form of particles having the shape of sphere, spheroid, or fiber. A fiber may have an aspect ratio (e.g., ratio of length to largest cross-sectional dimension perpendicular to the length) of greater than or equal to 2, greater than or equal to 5, greater than or equal to 10, and/or up to 50, up to 100, or greater. In some cases, a composition comprises a plurality of hydrogel particles, with solid forms of polypeptides (e.g., antibodies) at least partially encapsulated within the hydrogel particles. It has been observed that in some instances it is advantageous for the composition to be in the form of relatively small particles (e.g., microparticles or smaller). For example, in some embodiments where case of flowing the composition (e.g., by causing motion of a fluid, fluid suspension, or particulate composition in, through, or out of a container/vessel/conduit) is desirable (e.g., for subcutaneous administration), it may be advantageous for the composition to comprise relatively small particles of encapsulated polypeptides. In some embodiments, the hydrogel particles have an average largest cross-sectional dimension of greater than or equal to 50 nanometers, greater than or equal to 0.1 micron, greater than or equal to 0.2 microns, greater than or equal to 0.5 microns, greater than or equal to 1 micron, greater than or equal to 10 microns, greater than or equal to 30 microns, greater than or equal to 100 microns, or greater. In some embodiments, the hydrogel particles have an average largest cross-sectional dimension of less than or equal to 300 microns, less than or equal to 200 microns, less than or equal to 100 microns, less than or equal to 50 microns, less than or equal to 10 microns, less than or equal to 5 microns, less than or equal to 1 micron, less than or equal to 0.5 microns, or less. Combinations of these ranges are possible. For example, in some embodiments, the hydrogel particles have an average largest cross-sectional dimension of greater than or equal to 100 microns and less than or equal to 300 microns, greater than or equal to 30 microns and less than or equal to 200 microns, greater than or equal to 10 microns and less than or equal to 100 microns, greater than or equal to 1 micron and less than or equal to 10 microns, or greater than or equal to 50 nanometers and less than or equal to 1 micron. In some embodiments, the hydrogel particles have an average largest cross-sectional dimension of greater than or equal to 1 micron and less than or equal to 30 microns (e.g., greater than or equal to 1 micron and less than or equal to 5 microns, or greater than or equal to 10 microns and less than or equal to 30 microns). In some embodiments, the hydrogel particles have an average largest cross-sectional dimension of less than or equal to 1 micron. The largest cross-sectional dimension of a particle can be determined, for example, by analyzing microscopy images.

Hydrogels having the shapes and sizes described herein may be formed using any of a variety of suitable techniques that include, but are not limited to emulsion techniques (e.g., microfluidic emulsion, batch emulsion), extrusion (e.g., from a syringe/needle), spraying, and lithography. One example of an extrusion technique is centrifugal extrusion. Particle dimensions can be controlled by altering experimental parameters, for example, based on solution viscosities, flow rates, and the like. It has been observed, for example, that mixing conditions can affect the dimensions of the resulting hydrogel particles. In some embodiments, the hydrogel particles are formed at least in part via vortex mixing. In some embodiments, the hydrogel particles are formed at least in part via sonication. It has been observed that ultrasonication may, in some instances, provide relatively small hydrogel particles (e.g., hydrogel particles with largest cross-sectional diameters of less than or equal to 1 micron). It has also been observed that particle shape (e.g., aspect ratio) can be affected by collection distance (e.g., when using extrusion techniques) and/or centrifugation speed (when centrifugation techniques are used for hydrogel particle formation). It has also been observed that particle shape (e.g., aspect ratio) can be affected by chemical considerations such as cross-linking conditions (e.g., amount of crosslinking agent present) and/or molecular weight of hydrogel precursor components (e.g., molecular weight of polymer precursors).

In some embodiments, the solid form of the polypeptide (e.g., crystalline polypeptides, amorphous solids of polypeptides) are present in the composition in a relatively high amount. As mentioned above, having such a high loading of polypeptide in the composition may be advantageous in some instances. For example, a high concentration of polypeptide in the composition may allow for a given dose of a therapeutic or prophylactic to be delivered in a smaller volume than in instances with a lower concentration of polypeptide. Smaller volumes may, in some instances, be more convenient and comfortable for patients (e.g., when administered subcutaneously). Certain compositions (e.g., comprising carriers such as hydrogels) may allow for relatively high loadings while reducing or avoiding problems typically associated with high loadings, such as aggregation and/or poor flow properties. The loading of the solid form of the polypeptide in the composition may be expressed in any of a variety of suitable ways known to those of ordinary skill in the art. For example, one way the loading of the solid form of the polypeptide may be expressed is as a weight percentage as determined on a dry basis excluding the weight of the solvent. Another way of expressing the loading is as a volume percentage.

In some embodiments, the composition comprises the solid form of the polypeptide (e.g., crystalline polypeptides) in an amount of greater than or equal to 1 wt %, greater than or equal to 2 wt %, greater than or equal to 5 wt %, greater than or equal to 6 wt %, greater than or equal to 10 wt %, greater than or equal to 25 wt %, greater than or equal to 40 wt %, and/or up to 50 wt %, or greater. These weight percentages may be determined on a dry basis excluding weight of solvent. In some embodiments, the concentration of the solid form of the polypeptide (e.g., crystalline polypeptides) in the composition is greater than or equal to 1 mg/mL, greater than or equal to 2 mg/mL, greater than or equal to 5 mg/mL, greater than or equal to 10 mg/mL, greater than or equal to 20 mg/mL, greater than or equal to 50 mg/mL, greater than or equal to 100 mg/mL, greater than or equal to 150 mg/mL, greater than or equal to 200 mg/mL, or greater. In some embodiments, the concentration of the solid form of the polypeptide (e.g., crystalline polypeptides) in the composition is less than or equal to 500 mg/mL, less than or equal to 400 mg/mL, less than or equal to 330 mg/mL, less than or equal to 300 mg/mL, less than or equal to 250 mg/mL, or less. Combinations of these ranges are possible. For example, in some embodiments, the concentration of the solid form of the polypeptide (e.g., crystalline polypeptides) in the composition is greater than or equal to 1 mg/mL and less than or equal to 500 mg/mL, greater than or equal to 50 mg/mL and less than or equal to 330 mg/mL, or greater than or equal to 100 mg/mL and less than or equal to 300 mg/mL. The amount of polypeptide in the composition may be determined, for example, using thermogravimetric analysis (TGA) techniques.

As mentioned above, compositions described in this disclosure may have any of a variety of flow properties (e.g., rheological properties such as viscosity, shear-thinning, etc.) that can be beneficial for at least some applications (e.g., administration to a patient). Surprisingly, for example, it has been observed that compositions comprising relatively high concentrations of a solid form of a polypeptide can be prepared while having relatively low dynamic viscosity. Such an unexpected combination of polypeptide concentration and low dynamic viscosity may make some such compositions particularly suitable for administration for therapeutic and/or prophylactic applications, such as via injection (e.g., subcutaneous injection).

The dynamic viscosity of a composition generally refers to the composition's resistance to deformation at a given rate. Newtonian fluids have a dynamic viscosity independent of shear strain rate, while non-Newtonian fluids can present phenomena such as shear-thinning (where viscosity decreases with the rate of shear strain), shear-thickening (where viscosity increases with the rate of shear strain), and thixotropic fluids. In some, but not necessarily all embodiments, the compositions herein show non-Newtonian fluidic behavior. For example, in some embodiments, the compositions herein shear-thin. Shear-thinning may depend, for example, on the concentration of the polypeptide in the composition. Shear-thinning may be advantageous in some instances in which the compositions undergo relatively high shear stresses (e.g., injection).

In some embodiments, the compositions described herein demonstrate relatively low dynamic viscosity at a given shear rate. The dynamic viscosity of a composition (e.g., as a function of shear rate) can be determined by experimentally generating a flow curve for the composition using a rheometer. A parallel plate rheometer, as is known in the art, may be used to generate such flow curves, from which dynamic viscosity measurements can be made. An example of such a rheometer measurement is provided in the Examples below. In some embodiments, the composition has a dynamic viscosity of less than or equal to 0.3 Pascal seconds (Pa s), less than or equal to 0.2 Pas, less than or equal to 0.1 Pas, less than or equal to 0.05 Pas, less than or equal to 0.05 Pa s, and/or as low as 0.02 Pa s, as low as 0.01 Pas, or lower at a temperature of 25° C. and under a shear rate of greater than or equal to 10 s⁻¹, greater than or equal to 10 s⁻¹, greater than or equal to 10 s⁻¹, greater than or equal to 100 s⁻¹, greater than or equal to 500 s⁻¹, greater than or equal to 1,000 s⁻¹, and/or up to 2,000 s⁻¹, up to 4,000 s⁻¹, or greater. In some embodiments, the composition has a dynamic viscosity of less than or equal to 0.3 Pascal seconds (Pa s), less than or equal to 0.2 Pa s, less than or equal to 0.1 Pa s, less than or equal to 0.05 Pas, less than or equal to 0.05 Pa s, and/or as low as 0.02 Pa s, as low as 0.01 Pa s, or lower at a temperature of 25° C. and under a shear rate of 100 s⁻¹.

In some embodiments, a composition provided in this disclosure having a crystal comprising a solid form of a polypeptide has a dynamic viscosity that is lower than that of an aqueous suspension having an equivalent concentration of crystalline polypeptides under otherwise essentially identical conditions. Certain aspect of compositions described herein, such as encapsulation by a carrier material (e.g., a hydrogel), composition of the carrier, and dimensions of the carrier, may contribute to such a reduction in dynamic viscosity compared to free crystalline polypeptides in aqueous solutions. In this context, conditions may be essentially otherwise identical if parameters such as temperature, shear rate, instrument configuration, crystal morphology, and crystal size distribution (if comparing crystals) are kept essentially the same (e.g., within 5%, within 2%, within 1%, or closer), while the medium within which the crystals are present (e.g., encapsulation environment or lack thereof) is variable. For example, a batch of crystalline polypeptides may be prepared and separated into two sub-batches—a first of which is incorporated into a composition described herein (e.g., at least partially encapsulated by a hydrogel) and a second of which is suspended in an amount of aqueous solution resulting in an equal concentration of crystalline polypeptides as the inventive composition. The dynamic viscosity of the inventive composition and the comparative aqueous solution may then be determined using a rheometer under essentially identical parameters in terms of temperature and shear rate. The resulting dynamic viscosities may then be compared.

In some embodiments, a composition having a crystal comprising a solid form of a polypeptide has a dynamic viscosity that is at least 1.1 times, at least 1.2 times, at least 1.5 times, at least 2 times, at least 3 times, at least 4 times, and/or up to 4.5 times, up to 5 times, up to 5.2 times, up to 6 times, up to 8 times, up to 10 times lower than that of an aqueous suspension having an equivalent concentration of crystalline polypeptides under otherwise essentially identical conditions. It has been discovered herein that, surprisingly, such ratios of dynamic viscosity are achievable even for relatively concentrated compositions, such as those having a concentration of crystalline polypeptides of at least 1 mg/mL, at least 10 mg/mL, at least 50 mg/mL, at least 100 mg/mL, at least 200 mg/mL, and/or up to 300 mg/mL, up to 330 mg/mL, up to 500 mg/mL, or higher). In some embodiments, the above ranges in comparative reduction in dynamic viscosity is observed under at least one shear rate between 10 s⁻¹ and 4,000 s⁻¹ at 25° C. In some embodiments, the above ranges in comparative reduction in dynamic viscosity is observed under all shear rates between 10 s⁻¹ and 4,000 s⁻¹ at 25° C.

In some embodiments, a composition provided in this disclosure having a crystal comprising a solid form of a polypeptide has a dynamic viscosity that is lower than that of an aqueous suspension having an equivalent concentration of non-encapsulated non-crystalline polypeptides under otherwise essentially identical conditions. Non-encapsulated, non-crystalline polypeptides in an aqueous suspension may be at least partially dissolved, completely dissolved, or in a solid form suspended in the aqueous solution provided that they are not at least partially encapsulated (e.g., by a hydrogel).

In some embodiments, a composition having a crystal comprising a solid form of a polypeptide has a dynamic viscosity that is at least 50 times, at least 75 times, at least 100 times, and/or up to 500 times, or up to 1,000 times lower than that of an aqueous suspension having an equivalent concentration of non-encapsulated non-crystalline polypeptides under otherwise essentially identical conditions.

It has been observed that in some embodiments the compositions described release the polypeptides. For example, exposure of the compositions to dissolution conditions (in vivo or in vitro) may result in dissolution of the solid form of the polypeptide within or in proximity of the composition and ensuing separation of the polypeptide into bulk solution (e.g., via diffusion). It has further been observed the rate at which the polypeptide is released from the composition may depend on certain characteristics of the composition. For example, in some embodiments involving the at least partial encapsulation of a solid form of a polypeptide (e.g., as a crystal, as an amorphous solid) by a hydrogel (e.g., a hydrogel particle), certain characteristics of the hydrogel may affect the rate at which the polypeptide is released. Without wishing to be bound by any particular theory, some such hydrogel characteristics may include the pore size of the hydrogel, the chemical composition of the cross-linked polymer chains, the molecular weight of the polymer chains, the crosslink density of the hydrogel, the size of the hydrogel particles (when in particulate form), the concentration of porogens, the extent to which the hydrogel swells upon immersion in a dissolution medium (e.g., buffer solution or biological fluid), the extent and speed at which the hydrogel degrades in a dissolution medium, and the extent to which local viscosity increases within the hydrogel are observed upon immersion in the dissolution medium. The release rate may also depend at least in part on the size and/or concentration of the polypeptide. Adjustment of hydrogel characteristics (e.g., polymer chains, pore size, etc.) may allow for adjustment of release kinetics of the polypeptide. This may allow for tuning a release profile of a polypeptide for appropriate applications. For applications in which a fast release of polypeptide (e.g., antibody) is desired, hydrogels with relatively large pores may be employed. For applications in which a relatively slow release of polypeptide is desired, hydrogels with small pores, polymer chains having relatively high intermolecular interactions with the polypeptide, and/or a propensity for local viscosity increases may be employed. It has been observed that slower release may also be achieved via relatively high loadings of polypeptide in the composition.

In some embodiments, the composition releases the polypeptide at a relatively high rate. The release may be quantitatively determined using, for example, a time series of concentration measurements of the dissolution medium normalized by the amount of polypeptide initially incorporated in the sample. One way of performing the concentration measurements is using the Bradford protein assay method. The dissolution medium can be, for example, phosphate buffered saline (PBS) solution. In some embodiments, upon exposure to a phosphate buffered saline solution, greater than or equal to 70%, greater than or equal to 80%, greater than or equal to 90%, greater than or equal to 95%, greater than or equal to 99%, or more of the polypeptide is released into the solution 5 hours after the exposure. These percentages may be determined based on the fractional release of the polypeptide. These percentages may be determined at room temperature (25° C.).

In some embodiments, the composition releases the polypeptide at a relatively low rate. In some embodiments, upon exposure to a phosphate buffered saline solution, less than or equal to 90%, less than or equal to 85%, less than or equal to 80%, less than or equal to 75%, less than or equal to 70%, less than or equal to 65%, or less of the polypeptide is released into the solution 5 hours after the exposure. These percentages may be determined based on the fractional release of the polypeptide. These percentages may be determined at room temperature (25° C.).

While some compositions described comprise a solid form of a polypeptide as a therapeutic and/or prophylactic agent, additional therapeutic and/or prophylactic agents may be included in the compositions. For example, in some embodiments, the polypeptides are a first therapeutic and/or prophylactic agent, and the composition further comprises a second therapeutic and/or prophylactic agent. The second therapeutic and/or prophylactic agent may be a different type of polypeptide (e.g., protein such as an antibody, a therapeutic and/or prophylactic small molecule, etc.) Embodiments involving co-formulations with a first therapeutic and/or prophylactic agent and a second therapeutic and/or prophylactic agent (and optionally a third therapeutic agent and/or prophylactic agent, etc.) may include pharmaceutical cocktails. For example, a composition herein may be a cocktail comprising more than one therapeutic agents for treating infections such as HIV infection. As another example, it is known that there are a number of cancer indications for which there is a synergistic effect from having multiple different types of therapeutics in the same treatment. In some embodiments, a composition herein may include a multi-component cancer therapeutic in which one or more of the components is a solid form of a polypeptide at least partially encapsulated (e.g., by a hydrogel). In some embodiments, the second therapeutic and/or prophylactic may be reactive toward the polypeptide (e.g., when free in solution), but encapsulation of the polypeptide in solid (e.g., crystal form) may reduce or prevent reaction between the polypeptide and the second therapeutic and/or prophylactic agent (e.g., via sequestration). The second therapeutic and/or prophylactic agent may be present in the composition in a solid form (e.g., crystalline, amorphous solid) or dissolved in a solution (e.g., buffer). In some embodiments, the second therapeutic and/or prophylactic agent is also at least partially encapsulated by a carrier (e.g., hydrogel) of the composition. However, in some embodiments, the second therapeutic and/or prophylactic agent is present in the composition but is not encapsulated by the carrier (e.g., hydrogel).

The compositions described in this disclosure may be suitable for any of a variety of applications, as mentioned above. Surprisingly, it has been observed herein that some compositions described can be safe for administration to patients. The safety (e.g., biocompatibility) of a composition may depend at least in part on the composition of carriers (e.g., hydrogels) if used, the size of the particles, and the buffer used for forming the hydrogel.

In some embodiments, the compositions comprising solid forms of polypeptides described herein may be delivered to patients. Some such embodiments comprise administering to a patient the composition. Some such compositions may include a hydrogel and a solid form of the polypeptide, as described above. The polypeptide (e.g., a protein such as an antibody) may be at least partially encapsulated by the hydrogel. In some embodiments, the patient is a human patient. However, the patient may be an animal (e.g., a mammal other than a human) in some instances. Administration can be in vivo in some instances, or in vitro in other instances.

The compositions may be administered to the patient via any of a variety of techniques known in the art for medicinal administration. In some embodiments, the composition is administered to the patient via injection. For example, the composition may be injected as a bolus into the patient. Administration via injection can comprise subcutaneous, intramuscular, intravenous, intraperitoneal, intraosseous, intracardiac, intraarticular, and/or intracavernous injection using, for example, a hypodermic needle and a syringe. However, other types of administration are possible, such as via a patch, via inhalation, or via a patient implant. As mentioned above, it may be desirable to deliver relatively small volumes (e.g., less than or equal to 5 mL, less than or equal to 3 mL, less than or equal to 1.5 mL, less than or equal to 1 mL, or less) of compositions in certain types of injection, such as subcutaneous injections. Compositions comprising relatively high concentrations of polypeptide while maintaining good flow properties (e.g., relatively low viscosities) may therefore be desirable in some such instances.

Certain methods of administration may involve exposing the compositions to relatively high shear rates. For example, administering a composition via injection through an aperture such as a needle may expose the composition to high shear rates. Some embodiments may comprise injecting the composition into the patient by passing the composition through an aperture such that the composition experiences a shear rate of greater than or equal to 4000 s⁻¹, greater than or equal to 5000 s⁻¹, greater than or equal to 8000 s⁻¹, and/or up to 10,000 s⁻¹, up to 50,000 s⁻¹, up to 100,000 s⁻¹, or greater. Such high shear rates may be observed, for example, during the use of certain gauge needles. For example, the composition may be administered via injection by passing the composition through a needle having a gauge of greater than or equal to 20, greater than or equal to 23, greater than or equal to 25, greater than or equal to 26, and/or up to 28, up to 30, up to 31, or greater. It has been observed herein that some compositions (e.g., comprising hydrogels and solid forms of polypeptides) can maintain stability, integrity, and good flow properties at such high shear rates.

U.S. Provisional Patent Application No. 63/059,477, filed on Jul. 31, 2020, and entitled “Compositions Including Solid Forms of Polypeptides and Related Methods” is incorporated herein by reference in its entirety for all purposes.

The following examples are intended to illustrate certain embodiments of the present invention, but do not exemplify the full scope of the invention.

### Example 1

This Example describes experimentation and results related to compositions comprising polypeptides, according to certain embodiments.

### Introduction

Monoclonal antibodies (mAbs) are therapeutics known for their high specificity and versatility for the treatment of cancer and autoimmune disorders. Typically, mAbs are administered every few weeks via intravenous infusion in clinics with each administration requiring a few hours of time and the aid of a healthcare professional. The development of suitable formulations for subcutaneous injection of monoclonal antibodies is a significant therapeutic goal toward greater patient convenience and self-administration. For the subcutaneous route, these formulations can require a high concentration of mAbs (>>100 mg mL⁻¹) to meet volume requirements (<1.5 mL) for injection, although a formulation with such concentrations typically introduces additional challenges. At high concentrations, mAbs can self-associate and form clusters in solution, which manifests as high viscosity. Proactive strategies to engineer the viscosity of the formulation, such as changes in buffer conditions, addition of thinning excipients, or minor modifications to the mAb may be considered throughout development to avoid unacceptably high injection forces for administration. High concentration antibody solutions are also susceptible to accelerated protein degradation due to aggregation, which potentially impacts protein activity, pharmacokinetics, and safety. Small molecules drugs are commonly prepared in solid forms (e.g. amorphous solid dispersions, crystals) to impart the formulation with certain flow properties, greater solubility, enhanced stability, and tunable release properties. The crystalline form of proteins, while traditionally used for purification and structural characterization, can analogously be utilized to stabilize high concentration formulations of mAbs or other proteins. Crystals themselves are naturally densely packed with stable and folded protein at very high concentration (potentially >500 mg mL⁻¹). Further, some suspensions of protein crystals exhibit lower viscosities when compared to protein solutions of equivalent concentrations. Due to difficulties in developing protein crystal formulations (e.g. finding safe, suitable crystallization and stabilization conditions; scale-up of crystallization batch size), there has been limited commercial success outside of crystalline insulin, where crystals impart the formulations with long-lasting release. Consequently, there is significant room for development and innovation in this area.

Hydrogel materials are often studied as carriers for drug delivery due to their high water content, softness, and biocompatibility. Hydrogels can be produced with a variety of chemistries and microstructures, which can allow for design of hydrogels with diverse surface affinity and tunable drug release kinetics (e.g. fast release via hydrogel matrix degradation, slow release via diffusion). Certain existing works have exploited hydrogels for delivery of small molecule drugs in either the aqueous or crystalline form, and proteins in the aqueous form. These hydrogels are either formed in situ after injection by triggered gelation (e.g. pH, temperature), or formed beforehand for use as an oral formulation or implantable depot. Hydrogels can also be prepared as microsphere suspensions which exhibit lower viscosities when injected through a needle (i.e. high shear) and reach high volume fractions due to their ability to de-swell and deform when densely packed. It has been realized in the context of this disclosure that these properties make microsphere suspensions an interesting carrier to explore for a high concentration, low viscosity formulation.

Reported in this Example is a hydrogel/crystal microsphere formulation of a monoclonal antibody “mAb2”. The mAb2 was prepared as a concentrated suspension of crystals (>300 mg mL⁻¹) which was then encapsulated within hydrogel microspheres. The hydrogel microspheres were characterized to validate mAb2 crystallinity, mAb2 loading, and encapsulation efficiency. Further, in vitro dissolution experiments were conducted to demonstrate drug release from the hydrogel/crystal formulation. Finally, the flow curves of concentrated hydrogel/crystal microsphere suspensions demonstrate the improved flow properties of this formulation when compared to other forms of concentrated mAb2.

### Results and Discussion

### Production of Hydrogel/Crystal Microspheres

A hydrogel prepolymer was designed to polymerize under ultraviolet (UV) irradiation and to stabilize suspended mAb2 crystals (FIG. 3A). FIG. 3A shows that the hydrogel prepolymer is prepared by direct mixing of a concentrated suspension of mAb2 crystals in a PEG buffer, PEGDA and photoinitiator. FIG. 3B shows that the hydrogel prepolymer droplets were produced by using a microfluidic crossjunction; each droplet crosslinked by exposure to UV light. FIG. 3B shows that the crystals were well suspended within prepolymer droplets before crosslinking. After UV exposure, micron-scale crystals were trapped within the nanoporous matrix of the crosslinked microsphere. The schematic is not to scale.

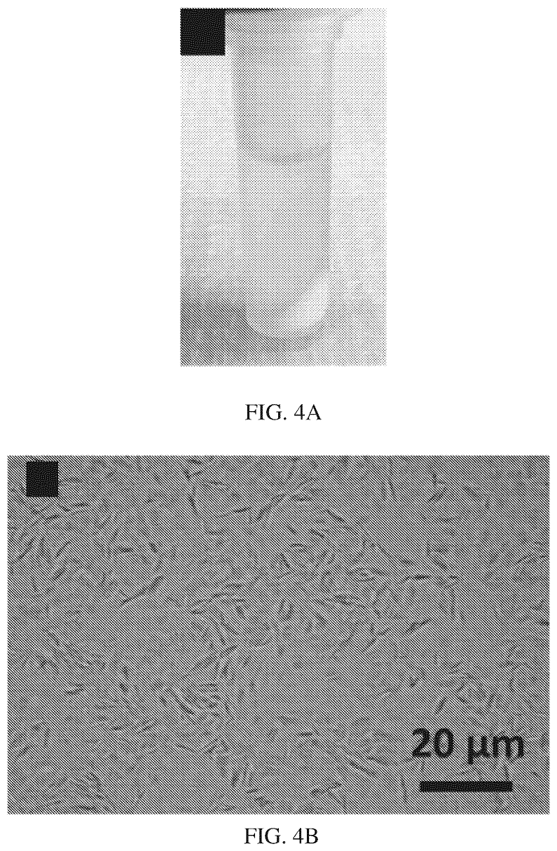

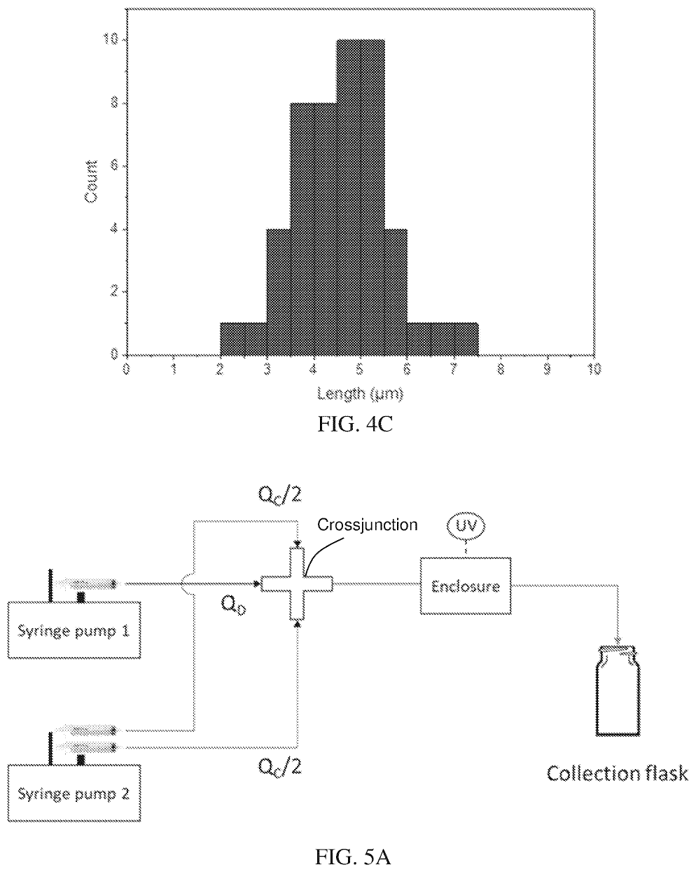

A concentrated suspension of mAb2 crystals in a 10% w/v poly(ethylene glycol) (PEG, MW 3350 Da), 50 mM HEPES, pH 7.0 stabilization buffer was first prepared (FIGS. 4A-4C) and then mixed with poly(ethylene glycol) diacrylate (PEGDA), a molecule which forms biocompatible hydrogels with predictable mesh size and degradability. FIGS. 4A-4C are images of the mAb2 crystal suspension. FIG. 4A shows the crystals separated from their crystallization buffer by sedimentation, which can be accelerated by centrifugation at 1700 RCF. FIG. 4B is a micrograph of mAb2 crystals. FIG. 4C is a size distribution of mAb2 crystals with an average length of 5 μm. Because mAb2 crystals were prepared and stabilized in a buffer containing a high concentration of PEG, it was anticipated that formulation in the presence of PEGDA would not significantly disrupt mAb2 crystallinity throughout processing. The amount of PEGDA and photoinitiator (Darocur 1173) were adjusted (10% and 1.5% v/v respectively) such that PEGDA rapidly polymerized (<60 s) when exposed to UV, and that the photoinitiator was fully soluble in the blend. The balance of the prepolymer blend (88.5% v/v) was the mAb2 crystals in their stabilization buffer. The PEG component of the buffer both stabilized the mAb2 crystals and induced the formation of interconnected pores within the polymerized hydrogel, which increased diffusion rates through the hydrogel.

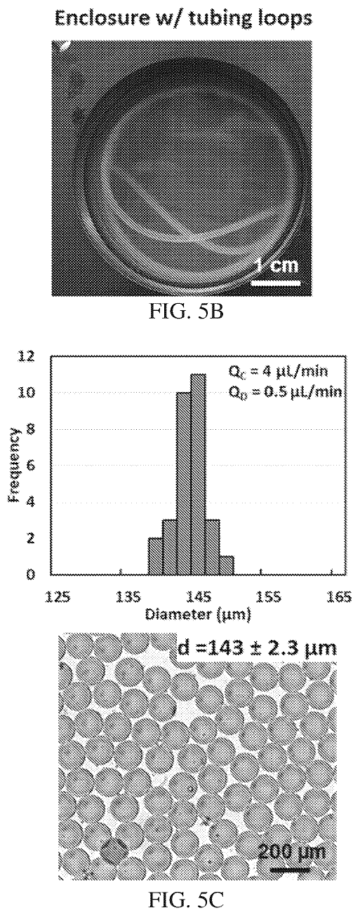

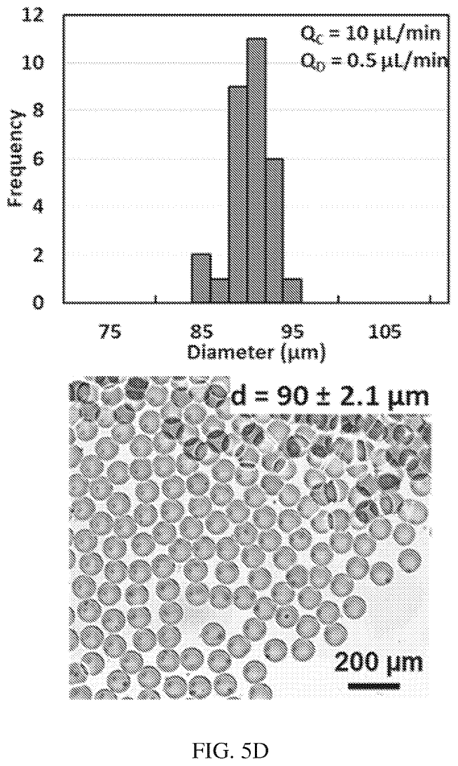

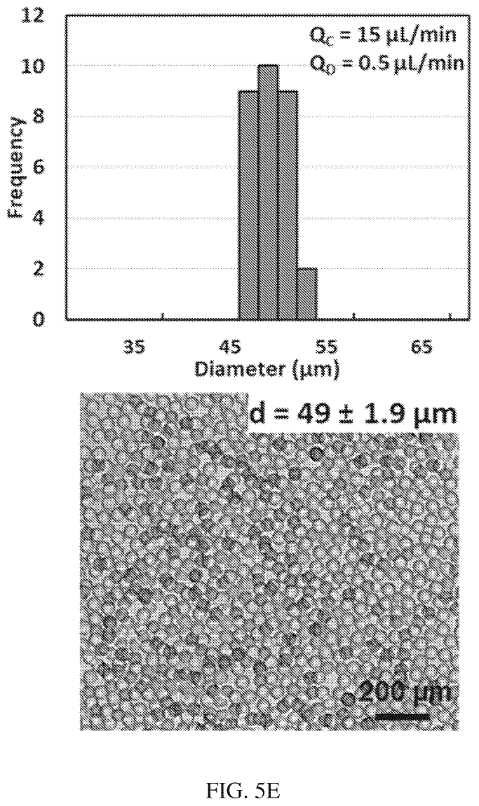

A simple microfluidic crossjunction and a UV LED were utilized to produce hydrogel/crystal microspheres as small as 30 μm in diameter (FIGS. 3A-3B and FIGS. 5A-5E). FIG. 5A is a schematic of the flow system, where Syringe pump 1 delivers dispersed phase (prepolymer) at flow rate Q_{D} and Syringe pump 2 delivers continuous phase (oil) at flow rate Q_{C}/2. At the crossjunction, the continuous phase impinges the dispersed phase and forms droplets, which photocrosslink downstream as they pass through the UV exposure enclosure. FIG. 5B is a photograph of the UV enclosure. The tubing was sandwiched between a lens cap and an optical diffuser, to which the mounted LED directly attached. FIGS. 5C-5E show characteristic microspheres produced with a prepolymer comprising 10% v/v PEGDA (MW 700 Da), 10% w/v PEG (MW 3350 Da) in 50 mM HEPES pH 7.0 at various flow rates.

The size and dispersity of hydrogel particles were influenced by flow rates of the oil and prepolymer, Q_{C} and Q_{D} (respectively), their viscosities μ_{C} and μ_{D}, and the interfacial tension. Microspheres were continuously produced at a rate Q_{D} of 0.1-1 μL min⁻¹ in an immiscible carrier fluid (mineral oil). Microsphere loading C_{load} was controlled by mixing a certain volume of concentrated mAb2 crystals into the prepolymer, and defined as:

$${C}_{load} = \frac{{v}_{m} {C}_{m}}{{v}_{t}} \qquad ( 1 )$$

where vₘ was the volume of mAb2 crystal suspension of concentration Cₘ, and vₜ was the final volume of mixed prepolymer. Microspheres with 50 μm in diameter (CV=0.04) were used for characterization at 50 and 100 mg mL⁻¹ mAb2 loadings. High concentration suspensions of mAb2 crystals exhibit high viscosities (>>0.1 Pa·s), and likewise, prepolymer solutions containing high concentrations of mAb2 crystals were also very viscous. For prepolymers with >200 mg mL⁻¹ mAb2, the microfluidic device yielded populations of smaller microspheres with higher polydispersity (30 μm diameter, CV=0.3).

### Characterization of Hydrogel/Crystal Microspheres

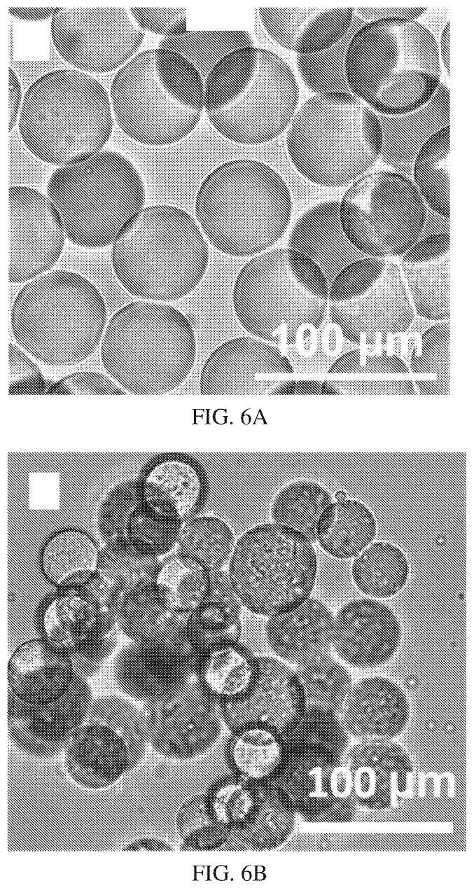

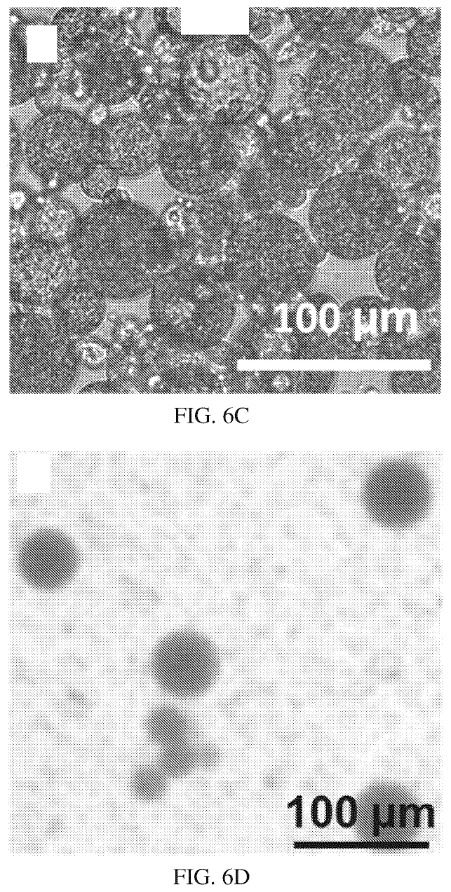

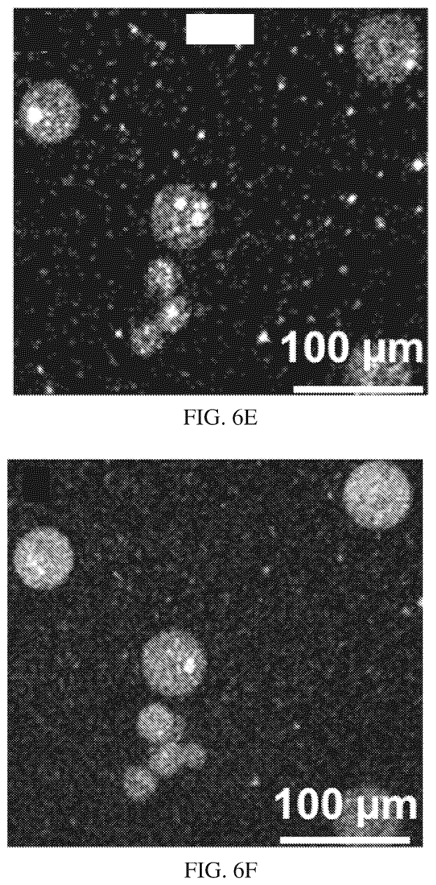

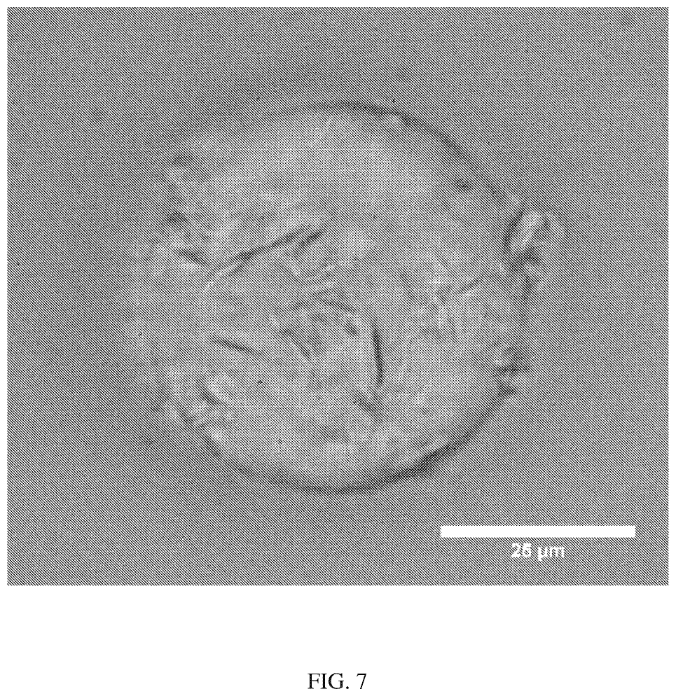

Hydrogel/crystal microspheres were spherical in shape and opaque with an apparently rough texture (FIGS. 6A-6F). The same sample of microspheres containing 200 mg mL⁻¹ of mAb2 crystals were imaged in bright field (FIG. 6D), second harmonic generation (SHG) (FIG. 6E), and ultraviolet two-photon excited fluorescence (UV-TPEF) (FIG. 6F). The features of small, needle-shaped mAb2 crystals could be distinguished within the microspheres under high magnification (FIG. 7). In FIG. 7, the shape of mAb2 crystals is distinguishable within the hydrogel. At high concentrations, individual crystals are difficult to identify visually due high sample opacity with traditional optical microscopy. Second harmonic generation (SHG) microscopy confirmed the presence of chiral crystals, and ultraviolet two-photon emission fluorescence (UV-TPEF) microscopy confirmed the presence of mAb2, which together confirmed that the hydrogel particles were packed with mAb2 crystals. The crystals were encapsulated and constrained within the hydrogel mesh, and they remained localized within hydrogels throughout polymerization and wash procedures without leakage. Further, the porous hydrogel enabled sufficient solvent access of PEG buffer to mAb2 crystals such that encapsulated material did not prematurely lose crystallinity or dissolve.

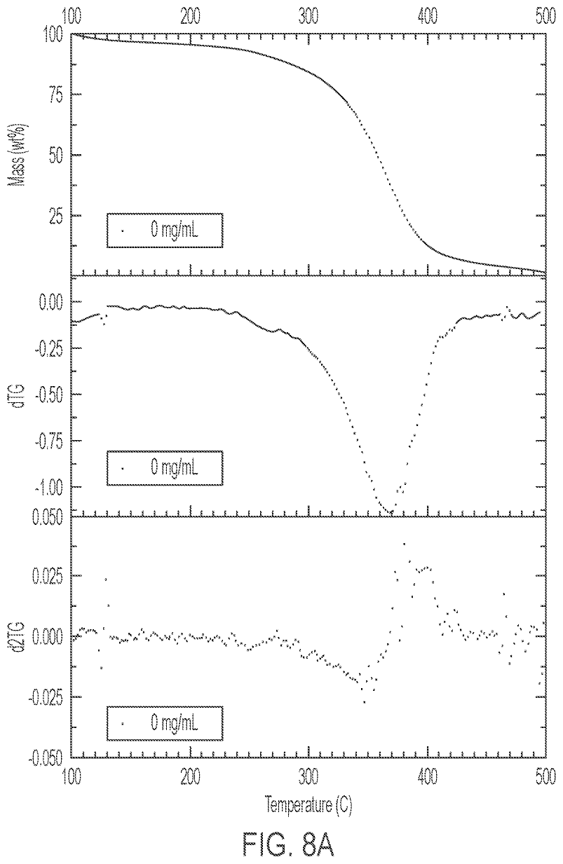

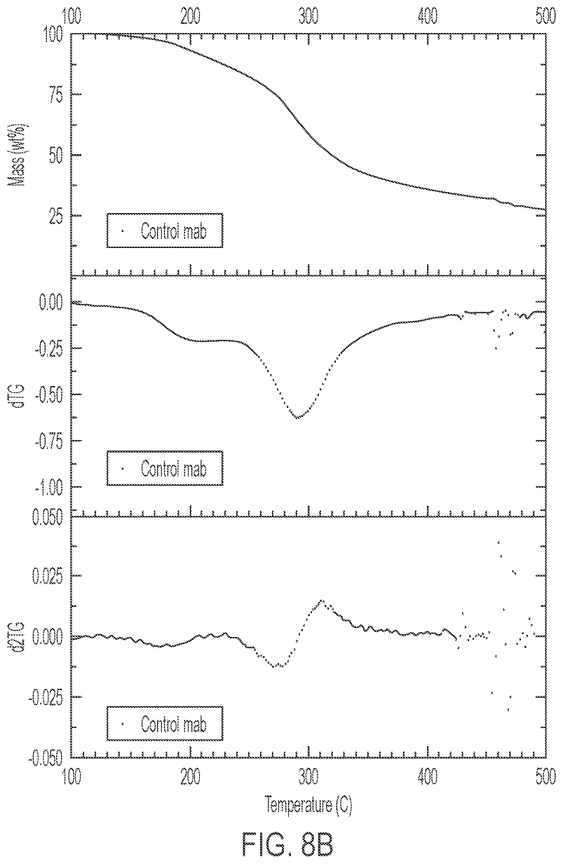

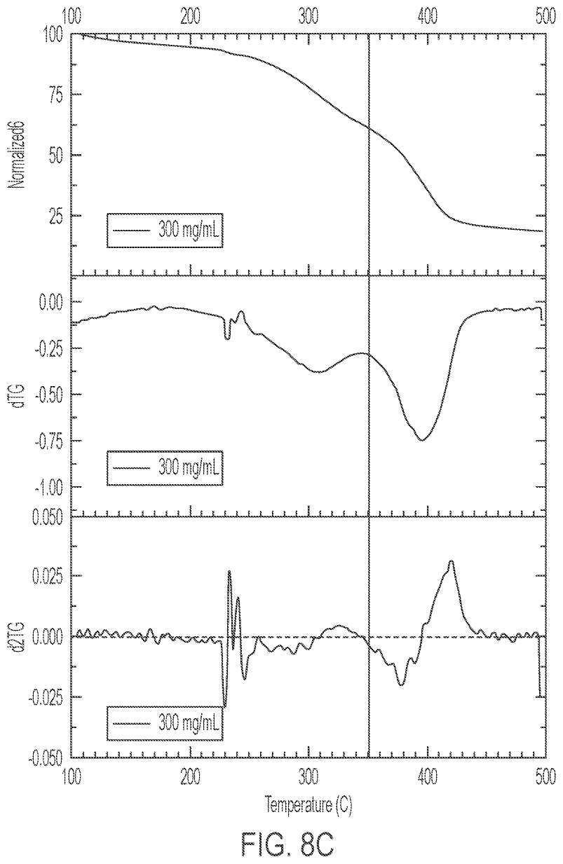

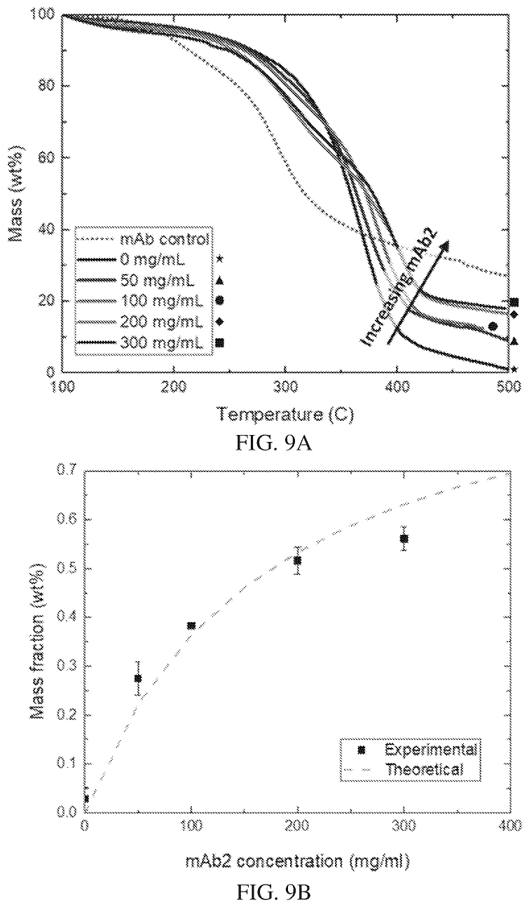

The loading of the mAb2 antibodies in the hydrogels was measured through thermogravimetric analysis. Control mAb2 decomposed over the temperature range 150-350° C., and a residual mass was present at 500° C. A PEG hydrogel control sample decomposed sharply between temperatures of 350-425° C. and was fully decomposed at 500° C. Details of the calculations and analysis are included FIGS. 8A-8C. FIG. 8A shows thermograms of hydrogel samples without mAb2. FIG. 8B shows thermograms of mAb2 without hydrogel. FIG. 8C shows thermograms of hydrogel-crystal composite samples. In the second differential thermogram, a zero is located at ˜350 C. corresponding to an inflection point in the data, indicating that hydrogel decomposition has become dominant over mAb2 decomposition. The inflection point is marked with a vertical line. Microspheres prepared with 50 mg mL⁻¹, 100 mg mL⁻¹, 200 mg mL⁻¹, and 300 mg mL⁻¹ of mAb2 were determined to have loadings of 27.5 wt %, 38.3 wt %, 51.6 wt % and 56.1 wt % respectively. 100% encapsulation was achieved for microsphere loadings <200 mg mL⁻¹, and 88% at 300 mg mL⁻¹ loading (FIGS. 9A-9B, Table 1 below). FIG. 9A shows the decomposition profiles of samples at microsphere loadings from 0-300 mg mL⁻¹. FIG. 9B shows the comparison of measured and theoretical values of mass for each hydrogel sample. Standard deviations are represented as error bars in FIGS. 9A-9B for three replicate samples.

TABLE 1
Dry loading, wet loading, and encapsulation
efficiency for hydrogel microspheres

| C_{load} (mg/mL) | w_{mAb2} (wt %) | w_{wet} (wt %) | Encapsulation efficiency |
|---|---|---|---|
| 50 | 27.5 | 6.0 | 100% |
| 100 | 38 | 11.7 | 100% |
| 200 | 51.6 | 22.3 | 97% |
| 300 | 56.1 | 30.2 | 89% |

The high encapsulation efficiency observed was attributed to the oil-in-water method used to sequester all crystalline material into droplets. Further, high loadings indicated that the mesh of the hydrogel was densely filled with mAb2.

In Vitro Release of mAb2 from Hydrogel Microspheres

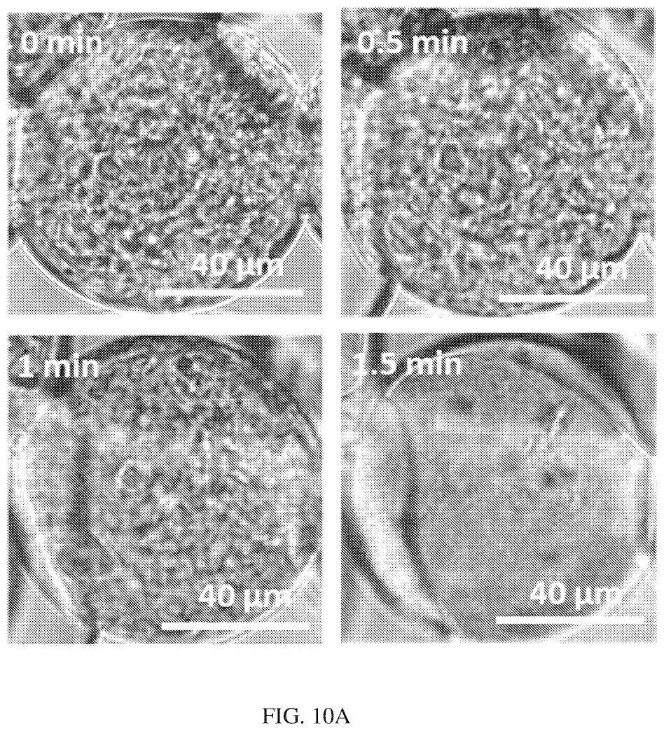

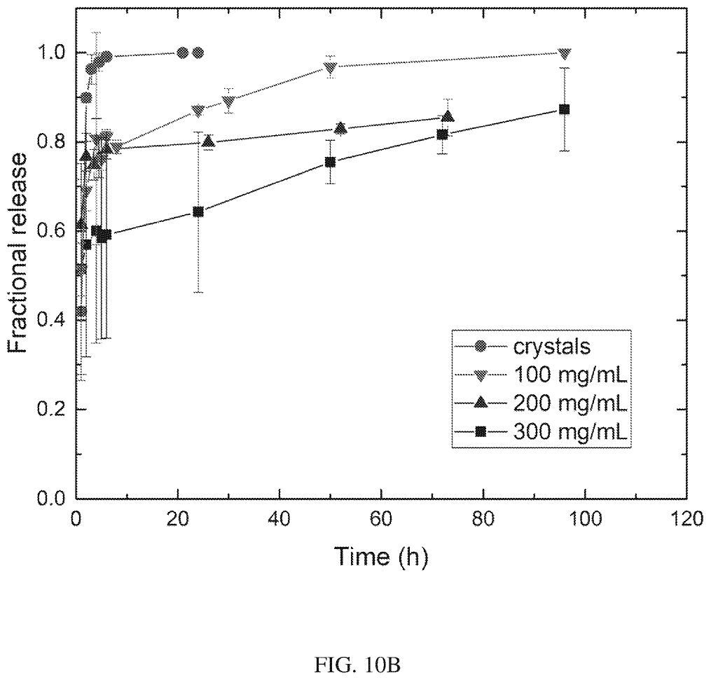

Hydrogel microspheres loaded with mAb2 crystals were immersed in phosphate buffered saline (PBS). It was observed that mAb2 dissolved from the crystals and released by diffusion through the porous polymer matrix, and the release rate was influenced by factors such as size of the antibody, polymer molecular weight, concentration of porogens, crosslink density, and size of the hydrogel particle. Within minutes, the appearance of the hydrogel microspheres changed from opaque to transparent and were no longer birefringent under crossed polarizers (FIG. 10A), indicating that the embedded crystals had dissolved. Interestingly, the dissolution profile indicated that after an initial burst release, mAb2 slowly released from hydrogels over for several hours to several days, with a slight dependence on the concentration of mAb2 encapsulated (FIG. 10B). FIG. 10A shows time-lapse imaging of crystal dissolution. The observed texture of the hydrogels evolved over 1.5 minutes until the microsphere is no longer opaque. FIG. 10B shows fractional release profiles from microspheres loaded with 100, 200, or 300 mg mL⁻¹ of mAb2. Standard deviations are represented as error bars for three replicate samples.

Without wishing to be bound by any particular theory, the burst release was attributed to heterogeneous crosslinking along the radius of a particles due to oxygen-inhibition of free radical polymerization and mild swelling of the hydrogels upon transfer to dissolution media. Further, the observation of rapid crystal dissolution and prolonged release indicated a two-step mechanism for the release of mAb2. First, the dissolution media rapidly penetrated the hydrogel microsphere, and diluted the stabilizing PEG buffer surrounding and within the crystals, which led to mAb2 crystal dissolution. The large mAb2 molecules (Dₕ˜11.1 nm by dynamic light scattering) then diffused through the porous hydrogel matrix over several days. The slower observed dissolution at high mAb2 concentrations likely arose from a local increase in viscosity within the hydrogel microspheres upon immersion and the dissolution of mAb2 crystals, which led to a suppression of the effective diffusion coefficient, although a complete investigation of release kinetics would be required to elucidate this phenomenon.

### Flow Curves of Concentrated Hydrogel & Crystal Suspensions

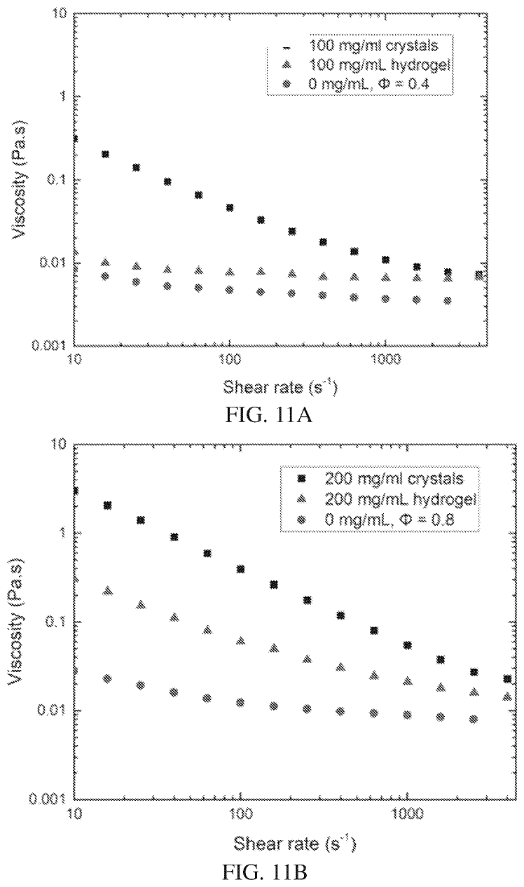

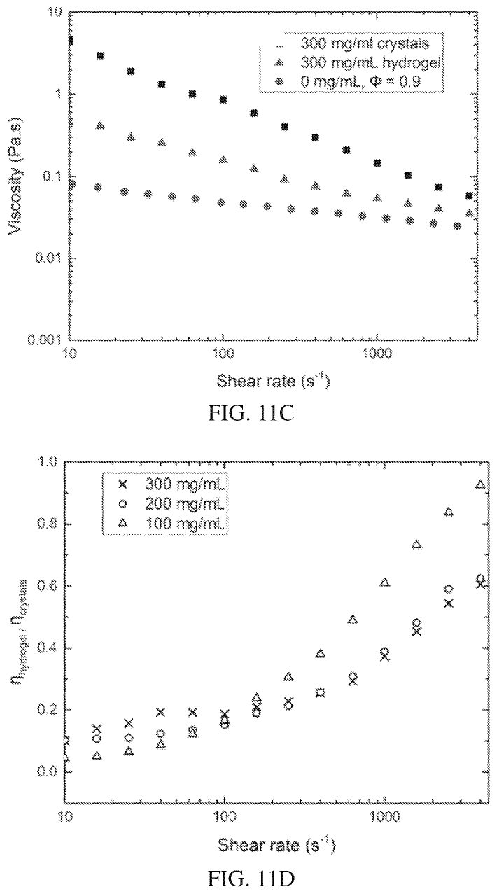

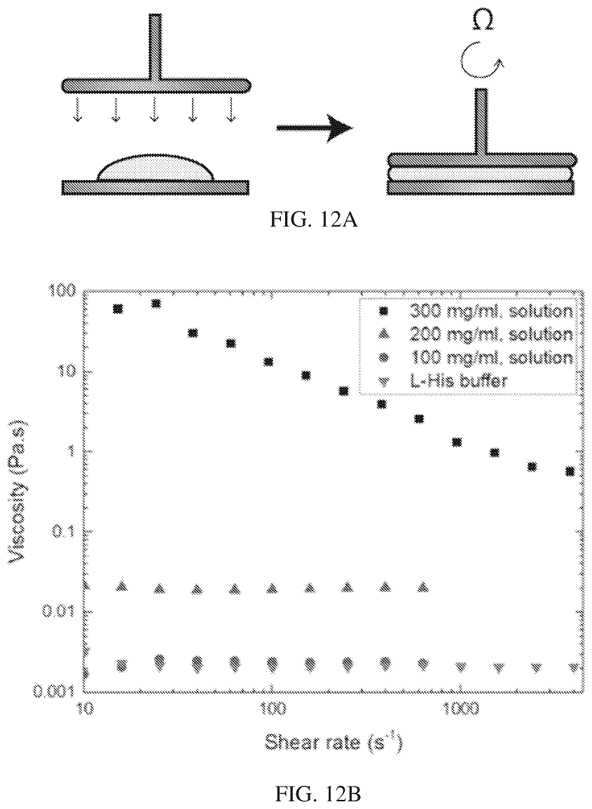

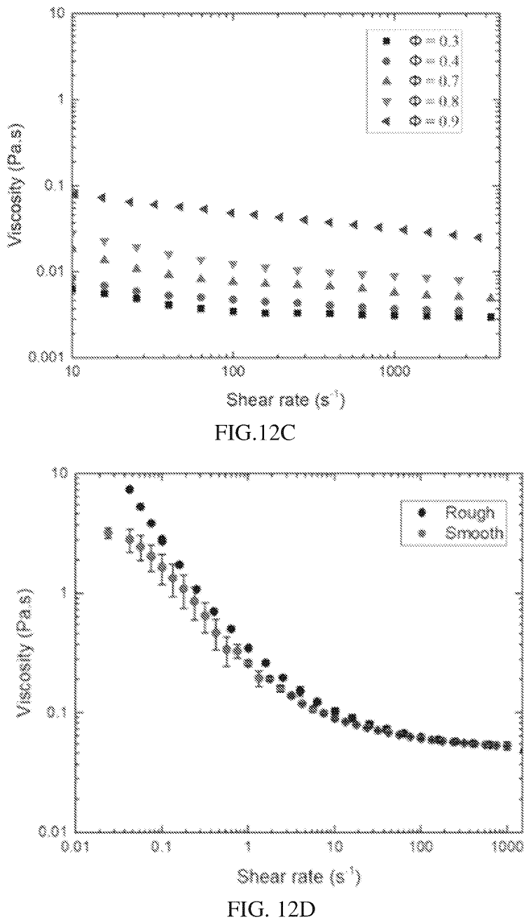

To evaluate how hydrogel/mAb2 crystal microspheres would perform in an injection, high loading microspheres were prepared as dense suspensions of microspheres for analysis of flow curves. Nominal particle volume fraction of hydrogel microspheres was defined as:

$$Φ = \frac{{v}_{t}}{{v}_{f}} \qquad ( 2 )$$

where vₜ was the volume of prepolymer converted into microspheres, and v_{f} was the final volume of the sample for rheometry. The formulated hydrogel load was defined as:

$${C}_{form} = {C}_{load} Φ \qquad ( 3 )$$

where C_{load} was the microsphere loading (Equation 1) and Φ was the nominal volume fraction of microspheres (Table 2 below). The nominal volume fraction of microspheres in each suspension was tuned to achieve a final formulated load to compare the hydrogel form to equivalent mAb2 dosages in either the crystal suspension form or concentrated solution form. For example, to prepare the most concentrated hydrogel formulation studied here of 300 mg mL⁻¹, microspheres were prepared with a microsphere loading of 333 mg mL⁻¹ mAb2 and centrifuged to a nominal particle volume fraction of 0.9. The rheometer gap size was set to 0.25 mm to approximate the inner diameter of a 26-gauge needle to optimize sample while reducing the potential flow effects of confinement. The gap size was at least 5× larger than the mean particle diameter. In the case of a subcutaneous injection where 1 mL is delivered in ˜10 s, the wall shear rate inside a 26-gauge needle is >100,000 s⁻¹. Due to sample and instrumentation limitations, flow curves were measured with a maximum shear rate of 4,000 s⁻¹. At this limit, the viscosities of hydrogel microspheres suspensions approached a viscosity plateau, and previous reports show that the viscosities of suspensions of soft particles often plateau and typically do not shear thicken. As a comparison, concentrated mAb2 solutions, mAb2 crystal slurries, and unloaded hydrogel microspheres were analyzed using the same experimental setup (FIGS. 11A-11D and FIGS. 12A-12D).

TABLE 2
Parameters for preparation of formulated loadings

| C_{form} (mg/mL) | Φ | C_{load} (mg/mL) |
|---|---|---|
| 100 | 0.4 | 250 |
| 200 | 0.8 | 250 |
| 300 | 0.9 | 333 |

FIGS. 11A-11D are plots of flow curves for mAb2 samples in the form of a suspension of non-encapsulated crystals (squares), hydrogel microspheres with encapsulated crystals (triangles), and a comparable volume fraction of hydrogel microsphere without mAb2 (circles). Viscosity was plotted against shear rate for formulated mAb2 concentrations of 100 mg mL⁻¹ (FIG. 11A), 200 mg mL⁻¹ (FIG. 11B), and 300 mg mL⁻¹ (FIG. 11C) suspended in a HEPES buffer at pH 7.0 containing 10% w/v PEG. FIG. 11D shows the viscosity reduction ratio for encapsulated crystals versus suspended crystals at each formulated loading.

FIG. 12A shows a schematic procedure for the rheometry measurements of using a Discovery Hybrid Rheometer 3 operated with constant angular velocity, and a parallel plate geometry with a 0.25 mm gap. FIG. 12B shows a flow curve of concentrated mAb2 solutions in 20 mM L-His, pH 5.4. FIG. 12C shows unloaded hydrogel particles at various nominal volume fractions suspended in 10% w/v PEG, 50 mM HEPES. FIG. 12D is a comparison of flow curves of a hydrogel sample with Ø=1.0 using a smooth plate (steel) and rough plate (240 grit, silica carbide). Suspensions of hydrogels may experience wall slip under shear conditions on the rheometer, which would reduce the measured viscosity; however, well above the yield stress, slip is negligible compared to the bulk flow, and the measured viscosity should approach the true viscosity at a given shear rate. This behavior was confirmed in PEGDA hydrogel microspheres at low shear rates (FIG. 12D), thus limiting the interpretation of viscosity to data collected in the high shear regime (>100 s⁻¹).

Solutions of mAb2 at 100 mg mL⁻¹ and 200 mg mL⁻¹ had a constant, low viscosity under shear. At 300 mg mL⁻¹ mAb2 solutions exhibited shear thinning and high viscosity (>0.6 Pa·s) (FIG. 12B). This behavior agreed with prior studies of antibody solutions that observed reversible self-association under shear which was attributed to mAb-mAb interactions and clustering at high concentrations. It was observed in this Example that mAb2 crystal slurries shear thinned at all concentrations measured, and at 200 mg mL⁻¹ and 300 mg mL⁻¹ had equivalent or lower viscosity than the corresponding solution form at a shear rate of 4000 s⁻¹. Unloaded hydrogel microsphere suspensions also shear thinned and plateaued at a viscosity that depended on the particle volume fraction (FIG. 12C). Hydrogel microspheres containing mAb2 crystals shear thinned, and interestingly their behavior was bounded by an equivalent volume fraction of unloaded hydrogel microspheres and an equivalently concentrated suspension of crystals across all shear rates (FIGS. 11A-11C). This behavior was consistent for 100, 200 and 300 mg mL⁻¹ formulated loads. Notably, the measured viscosity of the 300 mg mL⁻¹ formulation under shear was <0.035 Pa·s, an indication of potential suitability as an injectable formulation. In a qualitative injectability test, a hydrogel microsphere suspension with 300 mg mL⁻¹ formulated load was successfully ejected from a 26-gauge needle by hand without difficulty.

Without wishing to be bound by any particular theory, the improved flow behavior of the crystal-laden microspheres was rationalized to have arisen from three effects: (1) the hydrogel cloaked mAb-mAb interactions of embedded crystals, (2) the spherical microsphere shape minimized surface area-to-volume ratio of particles such that exposed (surface) mAb crystals have a smaller contribution to viscosity and (3) the hydrogel formulation was soft and deformable, resulting in enhanced flow behavior under shear. At low concentrations, the cloaking effect was most pronounced as the microspheres contained a low volume of crystals, and the flow properties of the hydrogel microsphere dominated. At high concentrations, a large volume of the hydrogel microsphere was occupied by mAb2 crystal, and thus the hydrogels were expected to behave effectively as a “spherical crystal” with lower viscosity than an equivalent mass of the freely suspended mAb2 crystals. In this report, all hydrogel formulations resulted in a decrease in viscosity relative to crystal suspensions (FIG. 11D). Notably, at a shear rate of 100 s⁻¹ the 300 mg mL⁻¹ hydrogel formulation had a 5.2-fold decrease in viscosity compared to the crystal suspension, and over a 50-fold decrease in viscosity compared to the concentrated free mAb2 solution.

### Conclusions

In summary, hydrogel microspheres containing monoclonal antibody crystals were produced with high loadings and low shear viscosity. It was demonstrated that maintaining these formulations in a PEG-rich buffer preserved the crystallinity of the mAb2 cargo, and that upon transfer to dissolution conditions, crystals dissolved and mAb2 released from the hydrogel matrix. When hydrogel/crystal microspheres were formulated as dense suspensions at high formulated loadings, they shear thinned and had lower viscosities than equivalent concentrations of mAb2 in crystal suspensions or free mAb2 solutions, demonstrating that crystal-loaded hydrogel microspheres may help overcome flowability issues for high concentration therapeutic dosages. While PEGDA was used to synthesize hydrogel microspheres in this study, the approach can be applied to many other hydrogel systems to further tune the release properties and performance of the formulation.

### Materials and Methods

Purified, humanized monoclonal antibody (mAb2) was provided by Merck & Co., Kenilworth, NJ, USA. Mineral oil, poly(ethylene glycol) diacrylate (PEGDA, molecular weight 700), 2-hydroxy-2-methylpropiophenone (Darocur 1173), sorbitane monooleate (Span 80), caffeine, and 4-(2-hydroxyethyl)-1-piperazineethanesulfonic acid (HEPES) were from Sigma-Aldrich Corporation. Poly(ethylene glycol) (PEG, molecular weight 3350) was from Hampton Research.

For crystallization, ‘PEG buffer’ was prepared as a 10% w/v PEG solution in 50 mM HEPES, pH 7.0. ‘Caffeine buffer’ was prepared as a 2.5% w/v caffeine solution in 20 mM L-His, pH 5.4. mAb2 was prepared at 40 mg mL⁻¹ in 20 mM L-His, pH 5.4. Solutions were prepared with distilled water and were sterile filtered with a 0.22 μm SUPOR filter (Acrodisc).

mAb2 Sequence Information

Table 3 includes the following sequence information for mAb2: complementarity-determining regions for variable domain of the light chain (VL-CDR1, VL-CDR2, VL-CDR3), variable domain of the light chain (VL), light chain, complementarity-determining regions for variable domain of the heavy chain (VH-CDR1, VH-CDR2, VH-CDR3), variable domain of the heavy chain (VH), and heavy chain.

TABLE 3
Sequence information for mAb2

| SEQ | | |
|---|---|---|
| ID | Descrip- | |
| NO | tion | Sequence |
| 1 | mAb2, | RASKGVSTSGYSYLH |
| | VL-CDR1 | |
| 2 | mAb2, | LASYLES |
| | VL-CDR2 | |
| 3 | mAb2, | QHSRDLPLT |
| | VL-CDR3 | |
| 4 | mAb2, | EIVLTQSPATLSLSPGERATLSCRASKGVST |
| | VL | SGYSYLHWYQQKPGQAPRLLIYLASYLESGV |
| | | PARFSGSGSGTDFTLTISSLEPEDFAVYYCQ |
| | | HSRDLPLTFGGGTKVEIK |
| 5 | mAb2, | EIVLTQSPATLSLSPGERATLSCRASKGVST |
| | light | SGYSYLHWYQQKPGQAPRLLIYLASYLESGV |
| | chain | PARFSGSGSGTDFTLTISSLEPEDFAVYYCQ |
| | | HSRDLPLTFGGGTKVEIKRTVAAPSVFIFPP |
| | | SDEQLKSGTASVVCLLNNFYPREAKVQWKVD |
| | | NALQSGNSQESVTEQDSKDSTYSLSSTLILS |
| | | KADYEKHKVYACEVTHQGLSSPVTKSFNRGE |
| | | C |
| 6 | mAb2, | NYYMY |
| | VH-CDR1 | |
| 7 | mAb2, | GINPSNGGTNFNEKFKN |
| | VH-CDR2 | |
| 8 | mAb2, | RDYRFDMGFDY |
| | VH-CDR3 | |
| 9 | mAb2, | QVQLVQSGVEVKKPGASVKVSCKASGYTFTN |
| | VH | YYMYWVRQAPGQGLEWMGGINPSNGGTNFNE |
| | | KFKNRVTLTIDSSTTTAYMELKSLQFDDTAV |
| | | YYCARRDYRFDMGFDYWGQGTTVTVSS |
| 10 | mAb2, | QVQLVQSGVEVKKPGASVKVSCKASGYTFTN |
| | heavy | YYMYWVRQAPGQGLEWMGGINPSNGGTNFNE |
| | chain | KFKNRVTLTTDSSTTTAYMELKSLQFDDTAV |
| | | YYCARRDYRFDMGFDYWGQGTTVTVSSASTK |
| | | GPSVFPLAPCSRSTSESTAALGCLVKDYFPE |
| | | PVTVSWNSGALTSGVHTFPAVLQSSGLYSLS |
| | | SVVTVPSSSLGTKTYTCNVDHKPSNTKVDKR |
| | | VESKYGPPCPPCPAPEFLGGPSVFLFPPKPK |
| | | DTLMISRTPEVTCVVVDVSQEDPEVQFNWYV |
| | | DGVEVHNAKIKPREEQFNSTYRVVSVLIVLH |
| | | QDWLNGKEYKCKVSNKGLPSSIEKTISKAKG |
| | | QPREPQVYTLPPSQEEMTKNQVSLTCLVKGF |
| | | YPSDIAVEWESNGQPENNYKTTPPVLDSDGS |
| | | FFLYSRLTVDKSRWQEGNVFSCSVMHEALHN |
| | | HYTQKSLSLSLGK |

Crystallization of mAb2

Crystals of mAb2 were grown in batches at the 2.5 mL scale, with each batch yielding about 30 mg of mAb2 in the crystalline form. For each batch, mAb2, PEG buffer, and caffeine buffer were combined at a volume ratio of 3:6:1. Crystallization mixtures were incubated at room temperature for 2 hours while rotating at 24 rpm on a rotisserie (Thermo Scientific, model 88881001). mAb2 crystals were recovered from the batches by centrifugation at 1700 RCF for 10 minutes (Eppendorf MiniSpin Plus), transferred into fresh PEG buffer, resuspended and stored at room temperature for up to 1 week prior to further processing.

Preparation of Prepolymer with mAb2 Crystals mAb2 crystal suspensions were concentrated through centrifugation at 1700 RCF. The crystal suspension was concentrated up to ˜333 mg mL⁻¹ (determined volumetrically) and then diluted to the desired mAb2 concentration by addition of PEG buffer. The prepolymer was prepared by direct addition of PEGDA and Darocur 1173 to the mAb2 crystal suspension, and then vortexed until the mixture was well dispersed.

### Microfluidic Formation of Microspheres

Prepolymer droplets were produced with a microfluidic apparatus consisting of two syringe pumps (PHD2000, Harvard Apparatus), a crossjunction (P-891, IDEX; 150 μm orifice), and transparent perfluoroalkoxy alkane tubing (PFA, 1902L, IDEX; OD 1/16″, ID 0.001″). Prepolymer was delivered to a single inlet of the crossjunction, and mineral oil was introduced via the 2 inlets oriented perpendicular to the prepolymer inlet. Droplet formation was controlled by modulating the continuous phase and prepolymer flow rates (Q_{C} and Q_{D}, respectively). Droplets were polymerized within the tubing downstream of the crossjunction outlet in a 2″ diameter cylindrical enclosure positioned in close contact with a UV LED (M365LP1, Thor Labs; 365 nm, 1150 mW). To accommodate higher flow rates, the tubing was coiled several times inside of the enclosure to increase time of exposure. Polymerized droplets were collected in a flask located downstream from the UV LED. Particles were produced with diameters ranging from 30-200 μm. Excess oil was removed from particle suspensions, and then samples were washed in fresh PEG buffer by vortexing for 30 s and centrifugation at 2000 RCF for 2 min at least 4 times to remove residual oil and unreacted hydrogel formers. For flow curve measurements, suspensions of crystal-loaded hydrogel microspheres were centrifuged at 1700 RCF to increase the nominal volume fraction to reach the target mAb2 loading.

mAb2 Loading and Encapsulation Efficiency

mAb2 content of hydrogels was measured using thermogravimetric analysis (Q500, TA Instruments). Approximately 5 mg of a microsphere suspension was transferred to sampling trays. Excess solvent was wicked from the samples, and they were further dehydrated at 100° C. under 25 mL min⁻¹ N₂ flow for 10 minutes prior to measurement. Samples were heated from 100° C. to 500° C., followed by an isothermal hold at 500° C. for 10 min. Sample mass was recorded continuously throughout the experiment from which drug loading and encapsulation efficiency were determined. The experiments were performed in triplicate with a 10° C. min⁻¹ temperature increase.

### Microscopic Characterization of Microspheres

Particle size distribution was evaluated using a Zeiss Axiovert microscope. A minimum of 30 particles were measured (ImageJ) for each sample for each reported mean diameter and coefficient of variation.

Second-order non-linear imaging of chiral crystals (SONICC, Formulatrix) was utilized to collect micrographs of microsphere samples in the following modes: bright-field, ultraviolet two-photon excited fluorescence (UV-TPEF), and second harmonic generation (SHG).

### In Vitro Dissolution

Microsphere samples were immersed in 1 mL of PBS and incubated on a rotisserie mixer at 24 rpm. At each sampling interval, the sample was centrifuged for 2 min at 1700 RCF and 0.5 mL of the supernatant was withdrawn and stored at 4° C. until analysis. 0.5 mL of fresh PBS was added to the dissolution sample and it was returned to the rotisserie. Concentration was determined by the Bradford method.

### Rheometry

Flow curves were measured using a DHR-3 rheometer (TA Instruments) with a steel parallel plate geometry (40 mm). A parallel plate was used to accommodate microspheres which are incompatible with a cone-plate geometry due to the small truncation length. The gap size was set to 0.25 mm, and >0.35 mL of sample was loaded for each measurement. The rheometer was operated with constant angular velocity and equilibrated for 20 s at each point. A correction was applied to account for the effect of inhomogeneous shear stress on non-Newtonian compositions. In the shear rate range tested (up to 4000 s⁻¹), all reported values were above the instrument's torque resolution and below shear rates at which effects of inertia and secondary flows become an issue.

Characteristics of mAb2 Crystal Suspensions

Batch crystallized mAb2 was centrifuged into a pellet at 1700 RCF then diluted and resuspended in 10% w/v PEG 3350, 50 mM HEPES pH 7.0 buffer. Within the suspension, crystals were intact needles with a typical length of 5 μm. The length and width of the crystals were 1 μm.

### Details for Microfluidic Particle Production

Hydrogel particles were synthesized using a microfluidic crossjunction coupled to a UV LED to initiate photocrosslinking. Monodisperse populations of particles can be produced as small as 50 μm.

### Mesh Size Estimation of Hydrogels Via Swelling

Bulk hydrogels composed of 10% v/v PEGDA (MW 700 Da), 8.8% w/v PEG (MW 3350 Da), 50 mM HEPES pH 7.0 were produced by photocrosslinking for 60 s exposure under a UV LED (M365LP1, ThorLabs) in the presence of 1.5% v/v Darocur 1173. Hydrogels were cut into 8×8×5 mm sections for swelling experiments. Each hydrogel section was soaked in an agitated bath of DI water for 24 h to fully swell and remove unreacted components. Each hydrogel was removed from the bath, patted dry to remove excess water, and then its swollen mass W_{swell} was recorded. The hydrogels were then dried for 48 h at 37° C. and the dry mass Wary was recorded. The mesh size of the hydrogels was estimated from the swelling Q by using Canal-Peppas theory, parameters determined by Merrill et al., and as applied by Cavallo et al. for photocrosslinked PEGDA hydrogels in A. Cavallo, M. Madaghiele, U. Masullo, M. G. Lionetto, A. Sannino, J. Appl. Polym. Sci. 2017, 134, 1, which is incorporated by reference herein in its entirety. Briefly, the swollen polymer volume fraction v_{2,s} was calculated from the swelling:

$$Q = \frac{{W}_{swell} - {W}_{dry}}{{W}_{dry}}$$

$${v}_{2 , s} = \frac{1}{Q \frac{ρ}{{ρ}_{H 2 O}} + 1}$$

where ρ was the density of PEGDA (1.12 g/mL) and ρ_{H2O} the density of water (1.0 g/mL). The average molecular weight between crosslinks Me was then estimated:

$$\frac{1}{{M}_{c}} = \frac{2}{{M}_{n}} - \frac{\frac{1}{{V}_{1}} [ ln ( 1 - {v}_{2 , s} ) + {v}_{2 , s} + χ {v}_{2 , s} ]}{ρ {v}_{2 , r} [ {( \frac{{v}_{2 , s}}{{v}_{2 , r}} )}^{\frac{1}{3}} - \frac{1}{2} ( \frac{{v}_{2 , s}}{{v}_{2 , r}} ) ]}$$

where Mₙ was the molecular weight of the monomer (700 Da), V₁ was the molar volume of water (18 mL/mol), χ was the Flory-Huggins solvent interaction parameter (0.426), and v_{2,r} the polymer volume fraction in the relaxed state (0.1, estimated as the volume fraction in the prepolymer). The average end-to-end distance between adjacent crosslinks (r⁻²)^{1/2} and the mesh size ξ of the hydrogel were calculated:

$${( {r}^{- 2} )}^{\frac{1}{2}} = l {( 2 \frac{{M}_{C}}{{M}_{r}} )}^{\frac{1}{2}} {C}_{n}^{\frac{1}{2}}$$

$$ξ = {( {r}^{- 2} )}^{\frac{1}{2}} {v}_{2 , s}^{- \frac{1}{3}}$$

where l was the bond length (1.5 Å), Mᵣ was the molecular weight of the PEG repeat unit (44 g/mol), and Cₙ was the characteristic ratio for PEG.

The calculated mesh size for 10% v/v PEGDA hydrogels was 2 nm. Notably, mAb2 molecules (Dₕ˜11.1 nm, DLS, Nanobook 90plus PALS) rapidly dissolved from the hydrogel, indicating that the mesh size of 2 nm for an unloaded hydrogel was a significant underestimate for the loaded hydrogels by at least an order of magnitude. It was suspected that mAb crystals (˜5 μm length) acted as a porogen and increase the porosity of the hydrogel, resulting in a larger, more interconnected porous network within the hydrogel.

Analysis of mAb2/Hydrogel Samples

An individual hydrogel was imaged under high magnification with a low content of mAb2 crystals, revealing that encapsulated mA2 crystals were visible and qualitatively intact. The loading of mAb2 in a given microsphere sample was determined by thermogravimetric analysis. The decomposition profiles of hydrogel and mAb2 slightly overlapped, but their peaks were distinguished in a composite sample where the majority of mAb2 degraded from 200-300° C., and hydrogel material from 320° C.-400° C. mAb2 left a residue (W_{residual}) while hydrogel fully decomposed by 500° C. To determine the transition between mAb2 and hydrogel decomposition, the second differential thermogram (d2TG) was utilized. At the zero of the d2TG, the data inflected and at this point it was approximated that hydrogel decomposition was the primary component decomposing. Using this point, the decomposed weight percent was separated into w_{d1} and w_{d2}, corresponding to mAb2 and hydrogel respectively. The weight percentage of mAb2 was calculated from TGA measurement:

W_{mAb2}=W_{d1}+W_{residual}

Expected weight percentage mAb2 loading was calculated from a mass balance:

$${w}_{expected} = \frac{{m}_{load}}{{m}_{load} + {m}_{PEG} + {m}_{PEGDA}}$$

Where m_{load} was the loaded mass of mAb2 in the crystal suspension, MPEG was the mass of PEG incorporated in the hydrogel, and PEGDA was the mass of the hydrogel material. Encapsulation efficiency was calculated as:

$$EE = \frac{{w}_{mAb 2}}{{w}_{expected}}$$

The loading was also estimated on a wet basis (i.e. for wet particles as in a suspension):

$${w}_{w e t} = \frac{{m}_{mAb 2}}{{m}_{mAb 2} + {m}_{PEG} + {m}_{PEGDA} + {m}_{water}}$$

Where m_{water} was the estimated water content, assuming: (1) the hydrogel particle had a fixed volume, (2) crystals contained 60% solvent by volume (based on a crystallographic Matthews Coefficient), and (3) the crystal density was 1.2 g/mL, similar to that of the crystallization buffer.

### Flow Curves of Control Samples

mAb2 solution in 20 mM L-His buffer was concentrated to 100, 200 and 300 mg/mL using a 50 kDa cutoff centrifugal filter. 100 and 200 mg/mL solutions behaved as Newtonian fluids with 0.0025 and 0.0195 Pa·s viscosities respectively, and at 300 mg/mL the solution exhibited a non-Newtonian response to shear with 0.58 Pa·s viscosity at a shear rate γ^{⋅}=4000 s⁻¹.

Bare (unloaded) hydrogels were prepared as concentrated suspensions in 10% PEG 3350, 50 mM HEPES pH 7.0 at several nominal volume fractions and exhibited mild shear-thinning. At nominal volume fractions of Φ=0.3, 0.4, 0.7, 0.8, and 0.9, the viscosities at γ^{⋅}=4000 s⁻¹ were 0.0030, 0.0035, 0.0049, 0.0080, 0.023 Pa·s respectively. The viscosity of the suspending buffer was 0.0026 Pa·s. Because the behavior of non-Newtonian fluids is dependent on the shear stress, and shear stress is a function of radius for parallel plate geometries, the Weissenberg-Rabinowitsch correction was applied for determining the shear rate and shear stress at the rim of the plate, and evaluating the viscosity η from the torque M and angular velocity Ω for a parallel plate of radius R and gap size h:

### Example 2

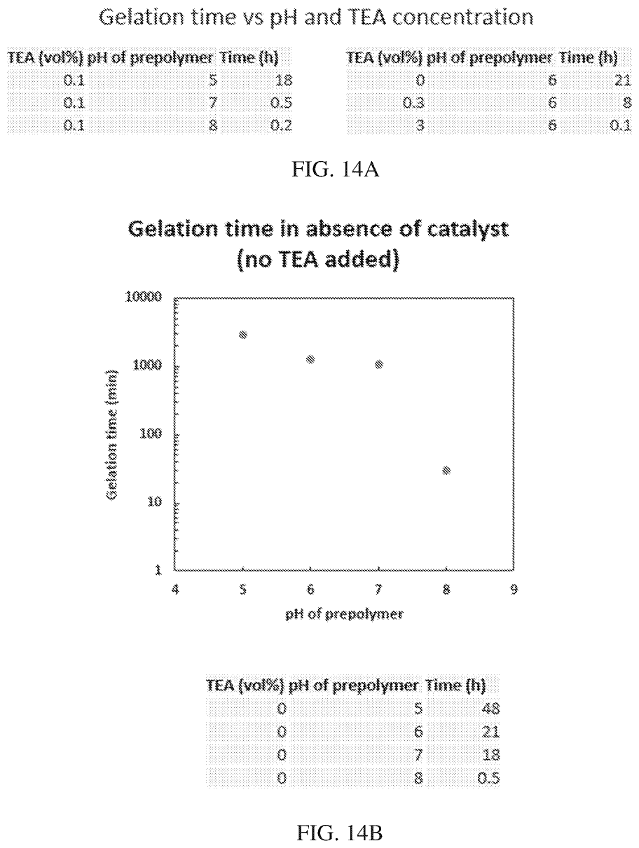

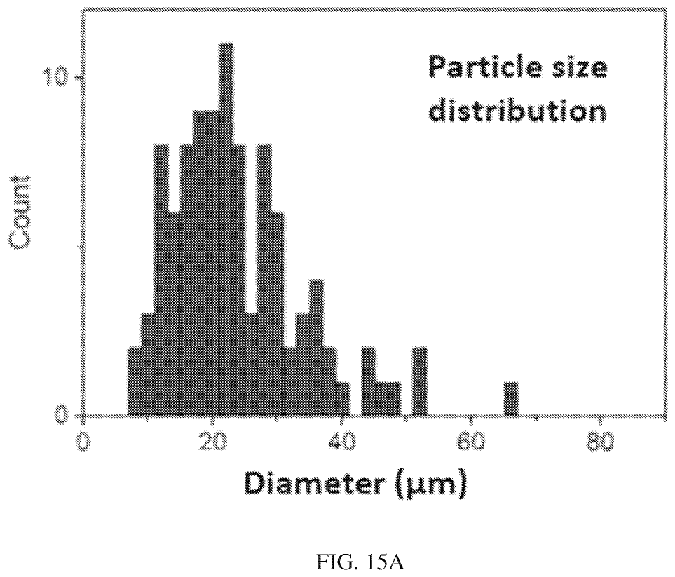

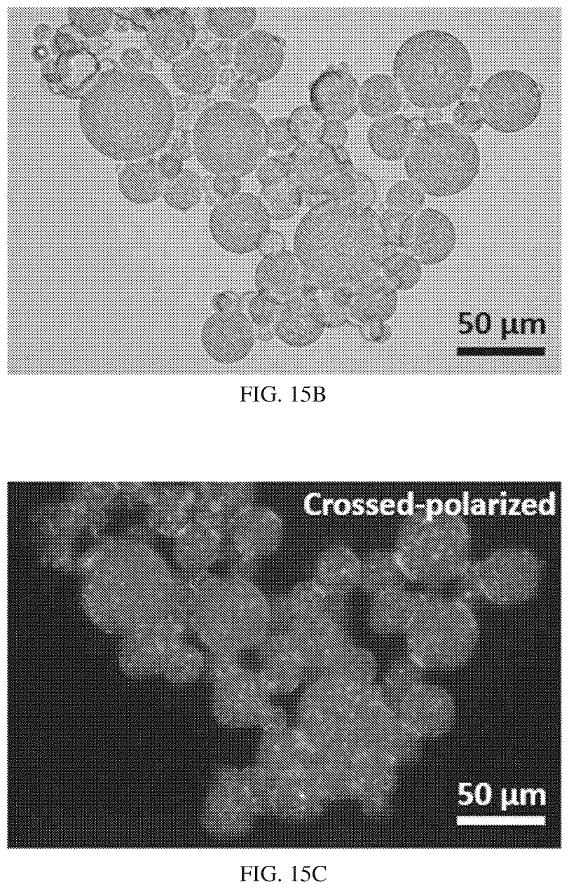

The following example describes a composition comprising a solid form of a polypeptide at least partially encapsulated by a hydrogel. A hydrogel comprising 4-arm poly(ethylene) glycol (PEG) monomers comprising vinylsulfone end-groups (FIG. 13A; PEG-VS. MW 10 kDa) covalently linked to one another by linear PEG molecules functionalized with thiol end-groups (FIG. 13C; PEG-DT, MW 3.4 kDa) was formed via thiol-Michael addition chemistry (FIG. 13D). The hydrogel crosslinked at a rate controlled by the pH of the solution (FIG. 14A,B), or instantaneously in the presence of an organic catalyst such as triethylamine (FIG. 13B; TEA). Hydrogel particles were prepared via a batch emulsification method. Prepolymer comprising 10% w/v PEG-VS/PEG-DT (3:2 mass ratio) in ‘PEG buffer’ (10% w/v PEG 3350, 50 mM HEPES, pH 5-8) and 100 mg/mL mAb2 crystals was added to an oil bath agitated at 200-2000 rpm with a stir bar until gelation completed (or, optionally, gelation was induced by addition of triethylamine via the oil phase). The resulting cross-linked microparticles were recovered by centrifugation and washed 4× with ‘PEG buffer’ to remove residual oil and excess reactants. This process produced microparticles with moderate polydispersity (FIG. 15A).

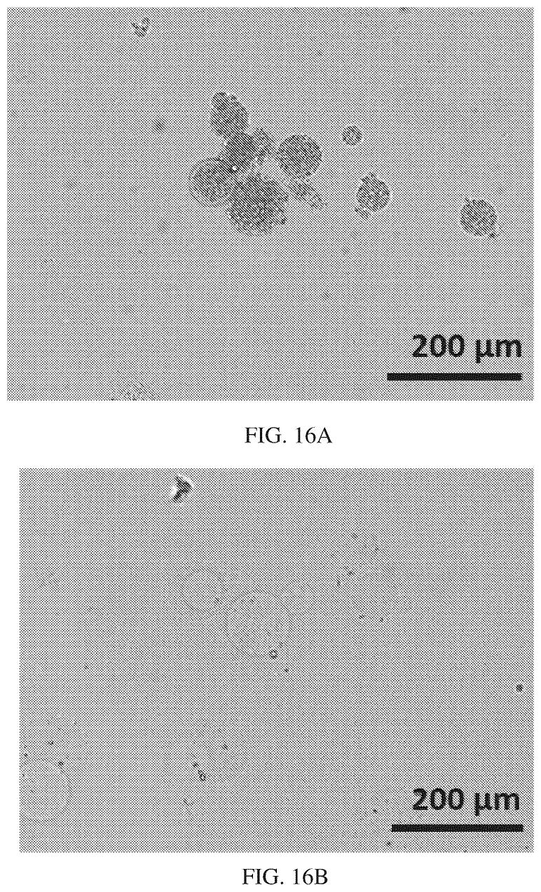

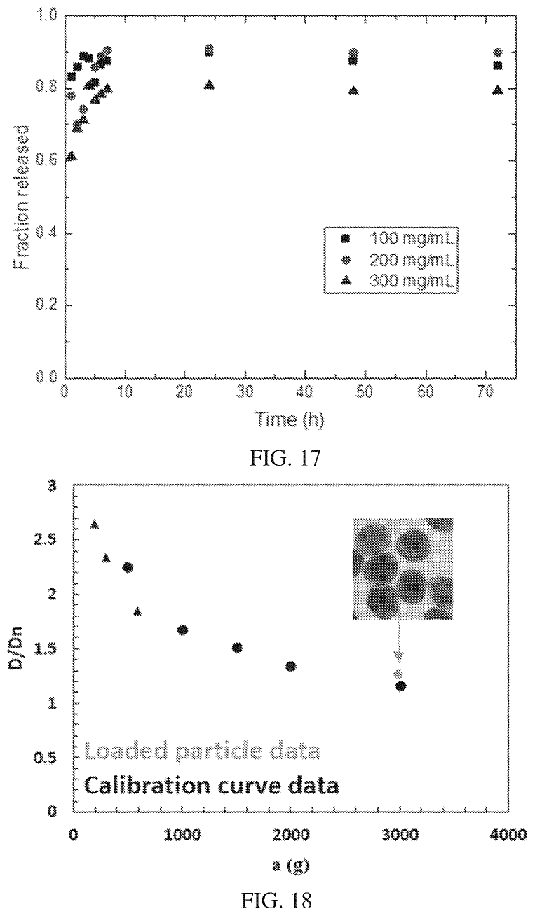

Crystal-loaded hydrogel microparticles were characterized by microscopy in bright field and with crossed polarizers. The microparticles were opaque in bright field (FIG. 15B), and bright between crossed-polarizers (FIG. 15C), indicating that the encapsulated material was likely crystalline. Upon immersion in a PBS pH 7.4 buffer, the particles clarified, indicating that the encapsulated crystals had dissolved (FIGS. 16A-B). Release was also measured over a time series and quantitated using the Bradford protein assay method, indicating that release completed within a few hours (FIG. 17).

### Example 3

The following example describes a composition comprising a solid form of a polypeptide at least partially encapsulated by a hydrogel. A hydrogel comprising a natural polysaccharide, alginate, was formed through ionic crosslinking. The hydrogel cross-linked in the presence of divalent cations, such as Ca²⁺. Hydrogel particles were prepared via a centrifugal extrusion method which deposited droplets into an aqueous bath containing a dissolved calcium salt. Prepolymer comprising 2% w/v sodium alginate in ‘PEG buffer’ (10% w/v PEG 3350, 50 mM HEPES, pH 7) and 200 mg/mL mAb2 crystals was centrifuged at 100-3000 RCF. The resulting cross-linked microparticles were recovered by centrifugation and washed 4× with ‘PEG buffer’ to remove excess calcium ions. This process produced microparticles with good monodispersity or bidispersity (FIG. 18).

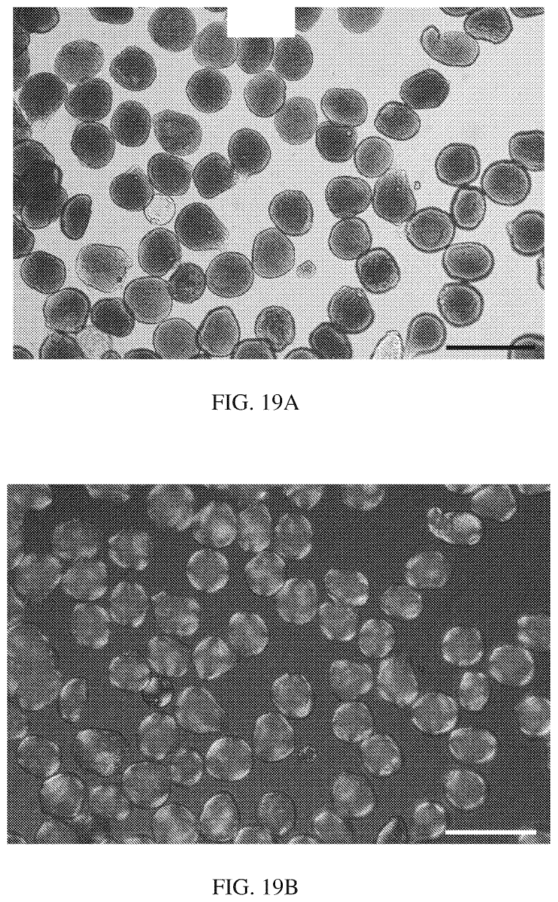

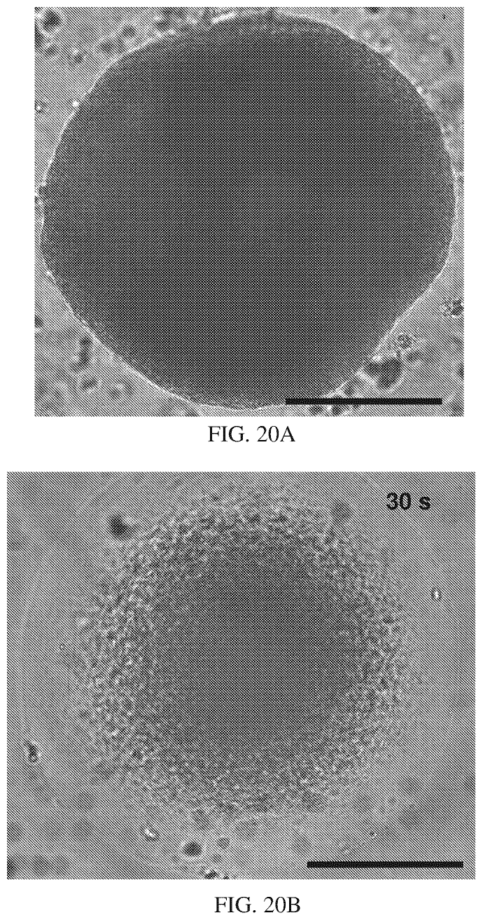

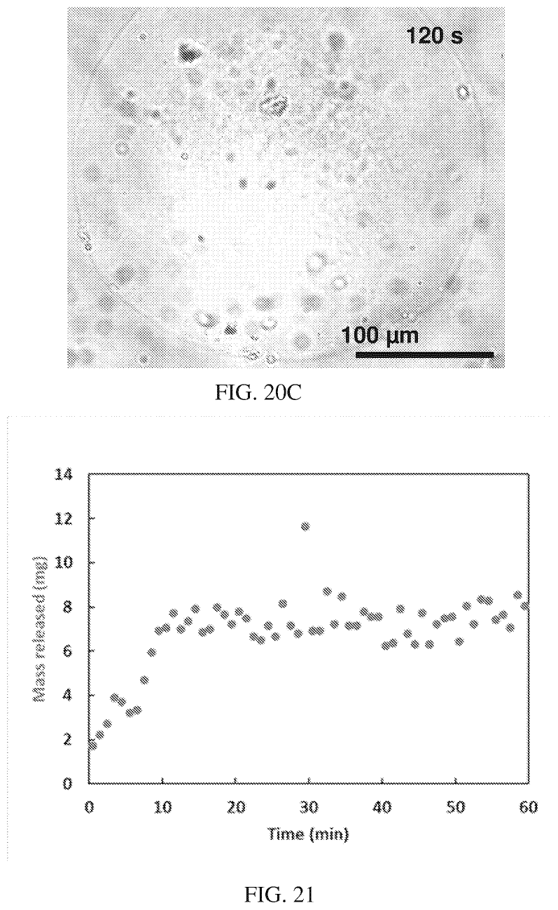

Crystal-loaded hydrogel microparticles were characterized by microscopy in bright field and with crossed polarizers. The microparticles were opaque in bright field (FIG. 19A), and bright between crossed-polarizers (FIG. 19B), indicating that the encapsulated material were likely crystalline. Upon immersion in a PBS pH 7.4, the particles clarified, indicating that the encapsulated crystals had dissolved (FIGS. 20A,B,C). FIG. 20A shows an alginate hydrogel microspheres immersed in PBS at t=0. FIG. 20B shows the alginate hydrogel microsphere immersed in PBS at t=30 s. FIG. 20C shows the alginate hydrogel microsphere immersed in PBS at t=120 s. The scale bars are in FIGS. 20A, B, C are 100 μm.

Release was also measured over a 120 minute time series and quantitated using a USP-2 dissolution apparatus with an in situ absorbance probe, indicating complete release in under an hour (FIG. 21).

### Prophetic Example 4

The following prophetic example describes an in vivo rat study of release of a polypeptide composition comprising a solid form of a polypeptide at least partially encapsulated by a hydrogel. It is noted that the hydrogel microspheres containing up to 56 wt % (dry basis) monoclonal antibody showed release within 4 days under in vitro dissolution conditions, as shown in FIG. 10D and described in Example 1. This release rate is relatively slower than the in vitro release rate of a few hours under similar in vitro dissolution conditions of a crystalline suspension of non-encapsulated mAb2 (also shown in FIG. 10D of Example 1). Based on the in vitro release data, a mAb2 crystal encapsulated by a hydrogel is expected to show protracted release and pharmacological activity in vivo in an animal model.

Using a rat model, mAb2 crystals encapsulated in hydrogel is dosed subcutaneously (SC) at 50 mg/kg. After a single injection, blood samples are drawn periodically over 7 days. A control formulation of mAb2 is used for intra venous (IV) administration. Blood levels of mAb2 are assayed and an area under the drug concentration time-curve (AUC), maximum concentration (Cₘₐₓ), minimum concentration (C trough), time of maximum concentration (Tₘₐₓ), and half-life of the antibody (t_{1/2}), are each assessed. A sample study design is provided below (Table 4). From the AUC comparison between IV and SC samples, absolute bioavailability is calculated:

Absolute bioavailability=AUC_{IV}/AUC_{SC}

TABLE 4
Rat Release Rate and Pharmacological Study Sample Design

| Group | Number of | Dose Level | | Administration |
|---|---|---|---|---|
| Number | Animals | (mg/kg) | Formulation | Route |
| 1 | 4 | 50 | Liquid | IV |
| 2 | 4 | 50 | mAb2 Crystal | SC |
| | | | Suspension | |
| 3 | 4 | 50 | mAb2 | SC |
| | | | Crystal- | |
| | | | encapsulated | |
| | | | Hydrogel | |

### Example 5

The following example describes a composition comprising a solid form of a polypeptide at least partially encapsulated by a hydrogel, and methods of tuning the hydrogel particle sizes. A hydrogel comprising 4-arm poly(ethylene) glycol (PEG) monomers comprising vinylsulfone end-groups (PEG-VS, MW 10 kDa) covalently linked to one another by linear PEG molecules functionalized with thiol end-groups (PEG-DT, MW 3.4 kDa) was formed via the thiol-Michael addition chemistry and buffers described in Example 2 above, and mAb2 crystals were encapsulated in the hydrogels. The size of the hydrogel particles encapsulating the mAb2 crystals was varied by tuning the presence of catalyst and the mixing conditions during particle formation. It was observed that the low shear method of vortex mixing can be used to create PEG-VS hydrogel particles laden with mAb2 crystals, with the hydrogel particles having diameters from 1 micron to 30 microns.

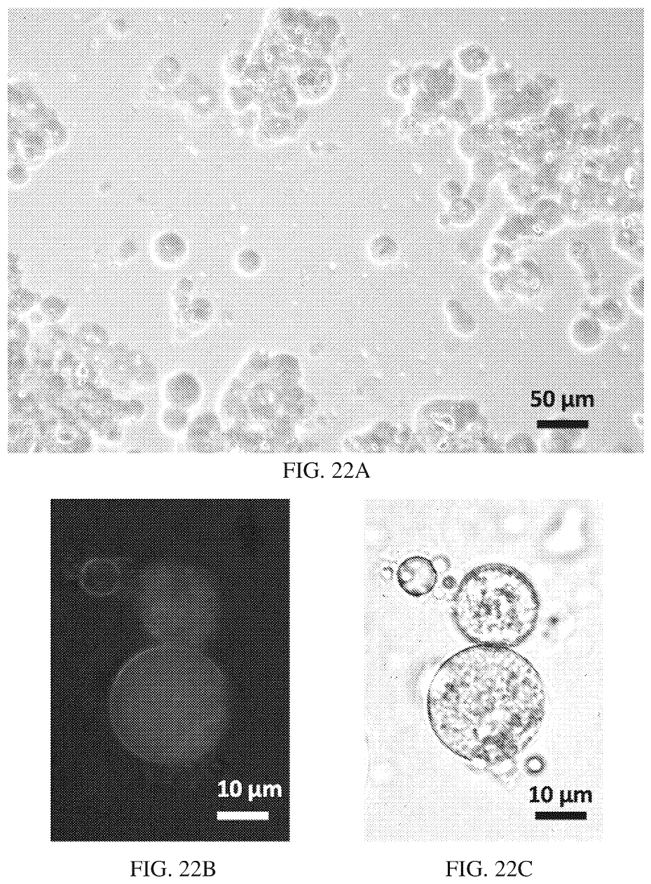

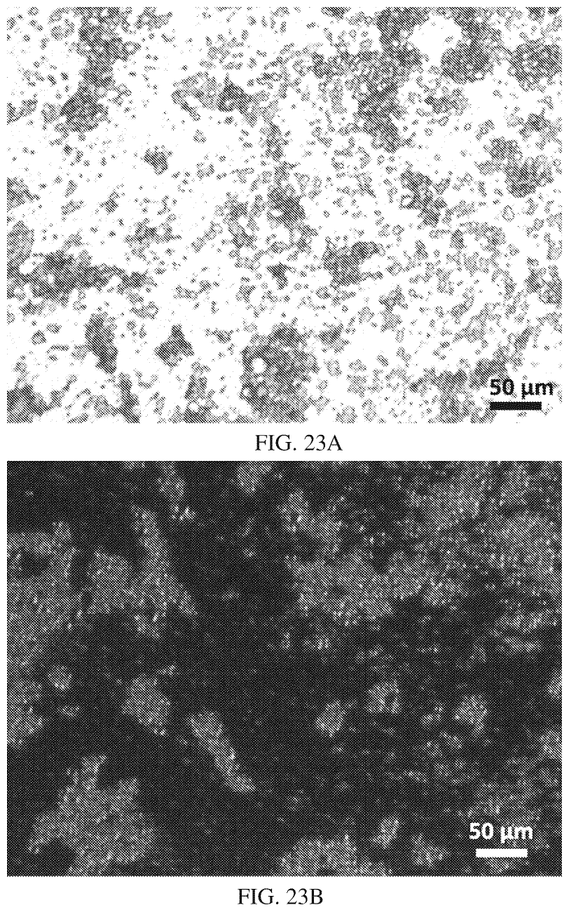

Crystal-loaded hydrogel microparticles formed under vortex mixing conditions and 0.05% v/v TEA were produced and characterized by microscopy in bright field and with crossed polarizers. FIGS. 22A-22C show bright field (FIGS. 22A and 22C) and crossed-polarized (FIG. 22B) microscopy images of resulting mAb2 crystal-laden PEG-VS hydrogel particles having diameters of 10-30 microns. FIGS. 23A-23B show bright field (FIG. 23A) and crossed-polarized (FIG. 23B) microscopy images of resulting mAb2 crystal-laden PEG-VS hydrogel particles having diameters of 1-5 microns.

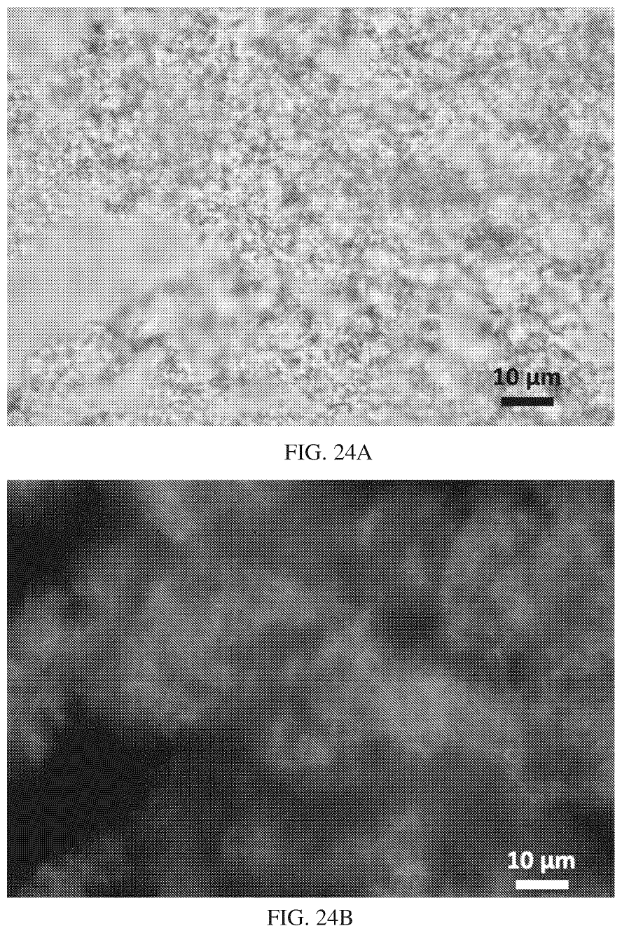

Crystal-loaded hydrogel microparticles formed under ultrasonication mixing conditions and no catalyst present were also produced and characterized by microscopy in bright field and with crossed polarizers. FIGS. 24A-24B show bright field (FIG. 24A) and crossed-polarized (FIG. 24B) microscopy images of resulting mAb2 crystal-laden PEG-VS hydrogel particles having diameters of less than 1 micron. As can be seen in FIGS. 24A-24B, the mAb2 crystals can be partially encapsuled in these small particles and/or can bridge the small particles together. The crossed-polarized images suggest the existence of the crystals within these hydrogel particles.

### Example 6

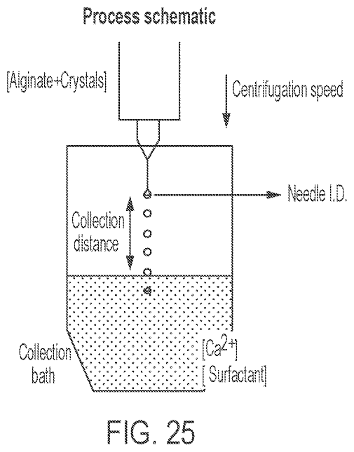

The following example describes a composition comprising a solid form of a polypeptide at least partially encapsulated by a hydrogel. A hydrogel comprising a natural polysaccharide, alginate, was formed through ionic crosslinking. The hydrogel cross-linked in the presence of the divalent cation Ca²⁺. The hydrogel particles were prepared via a centrifugal extrusion method in which droplets of prepolymer were deposited into an aqueous bath containing a dissolved calcium salt. FIG. 25 shows a schematic process diagram of the experimental setup for the centrifugal extrusion method. The process in which the particles were manufactured was tuned to generate injectable particles (spherical, smaller, softer particles) with relatively high encapsulation efficiency (due to smaller loss of mAb2 in the bath) with manufacturing considerations (desiring in some instances a fast process). It was determined in the context of this disclosure that manufacturing alginate crystal hydrogels by extrusion posed distinct challenges. It was determined that the presence of shear-thinning crystals affected hydrogel particle formation, and non-spherical particle shapes could, in some instances, affect performance properties related to flowing hydrogel microparticle suspensions. Additionally, it was determined that crystalline monoclonal antibodies can benefit from specific conditions to reduce or prevent premature dissolution throughout processing. To overcome these challenges specific considerations were considered.

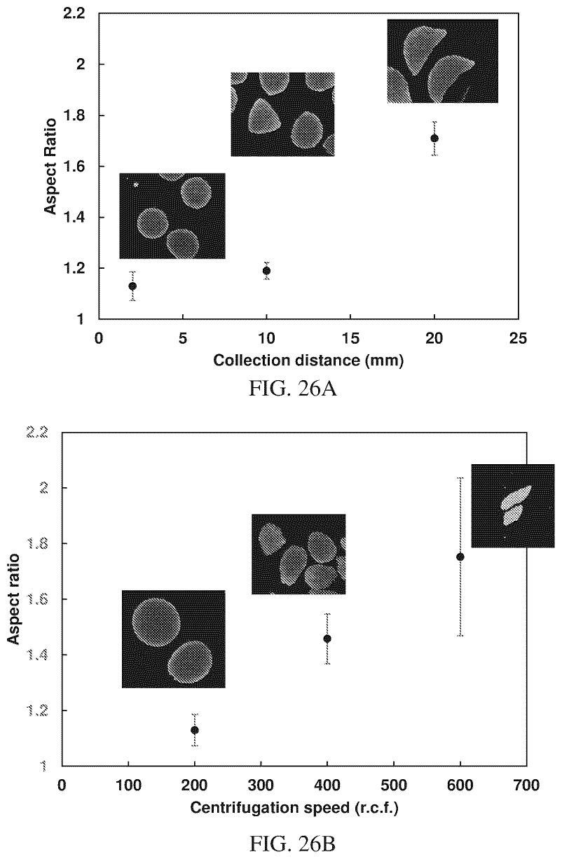

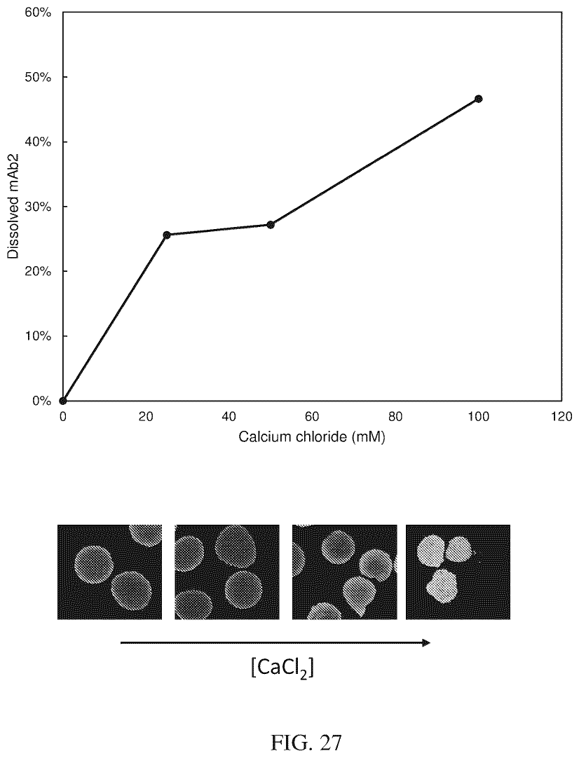

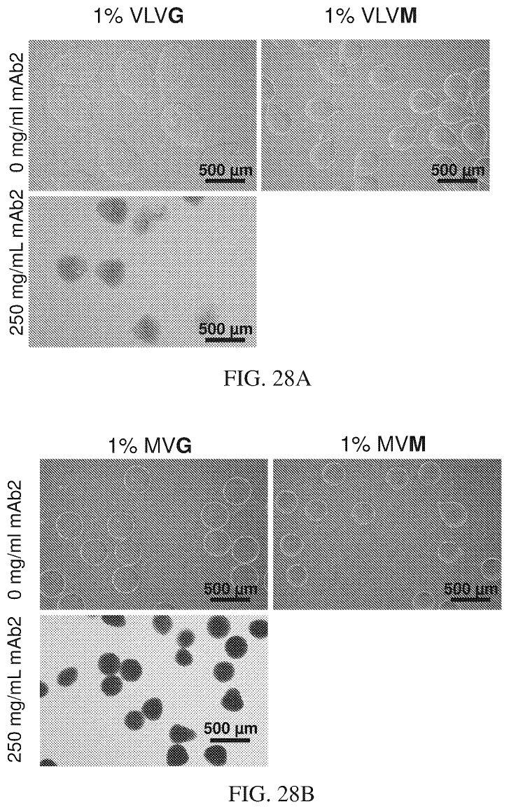

FIGS. 26A-26B provide data illustrating physical considerations in the particle manufacturing process, while FIGS. 27-28B illustrate chemical considerations. As FIG. 26A indicates, the collection distance (shown in FIG. 25) affected particle shape, and particles can flatten as collection distance increases. It is believed that the observed flattening was due to greater droplet velocities at higher collection distances. It was also determined that the centrifugation force and the flow rate can also be controlled to reduce or prevent jetting of the hydrogel particles. As can be seen in FIG. 26B, the centrifugation speed affects particle morphology.

FIG. 27 shows data for antibody crystal dissolution versus concentration of calcium chloride (CaCl₂)) in the aqueous solution that receives the droplets (top), and associated images of the resulting particles with increasing CaCl₂) concentration (bottom). FIG. 27 illustrates the effect of Ca²⁺ excess concentration on disturbing the antibody crystal stability and resulting dissolution of the crystal. Furthermore, it was observed that the hydrogel particles developed teardrop shapes at higher Ca²⁺ concentrations. It was determined based on the results that for at least some embodiments, Ca⁺² concentrations of 5-20 mM can be advantageous for achieving desired formulations.

Four different types of alginate formulations were tested in to determine the effect of polymer chain viscosity (e.g., alginate viscosity) and molecular weight on the hydrogel particles. The first type was VLVM (very low viscosity (<20 mPa sec; MW<75 kDa) alginate high mannuronic acid), and the second type was VLVG (very low viscosity (<20 mPa sec; MW<75 kDa) alginate high guluronic acid), the third type was MVG (medium viscosity (>200 mPa sec; MW>200 kDa) alginate high mannuronic acid), and the fourth type was MVM (medium viscosity (>200 mPa sec; MW>200 kDa) alginate high guluronic acid). FIG. 28A shows microscopy images of hydrogels of 1% VLVG and VLVM in the absence of mAb2 crystals (top) and hydrogels of 1% VLVG with 250 mg/mL of encapsulated mAb2 crystals (bottom). FIG. 28B shows microscopy images of hydrogels of 1% MVG and MVM in the absence of mAb2 crystals (top) and hydrogels of 1% MVG with 250 mg/mL of encapsulated mAb2 crystals (bottom). As demonstrated in these figures, the methods described in this disclosure can be used to generate injectable hydrogels with alginate concentrations as low as 1% and viscosities as low as 20 mPa sec or lower, and that the viscosity can affect the shapes of the hydrogel particles.

### Example 7

This example describes testing of the injection performance and stability of a composition comprising a solid form of a polypeptide at least partially encapsulated by a hydrogel. Alginate hydrogel particles containing mAb2 crystals were prepared according to the procedure described in Example 3. Suspensions comprising the mAb2 crystal-laden alginate hydrogel particles with mAb2 crystal loadings of either 50 mg/mL or 200 mg/mL were loaded into syringes and were observed to have favorable flow properties when ejected from subcutaneous needles. It was observed that the stability and potency of the mAb2 polypeptides were unperturbed when encapsulated and released from the alginate hydrogels.

### Example 8

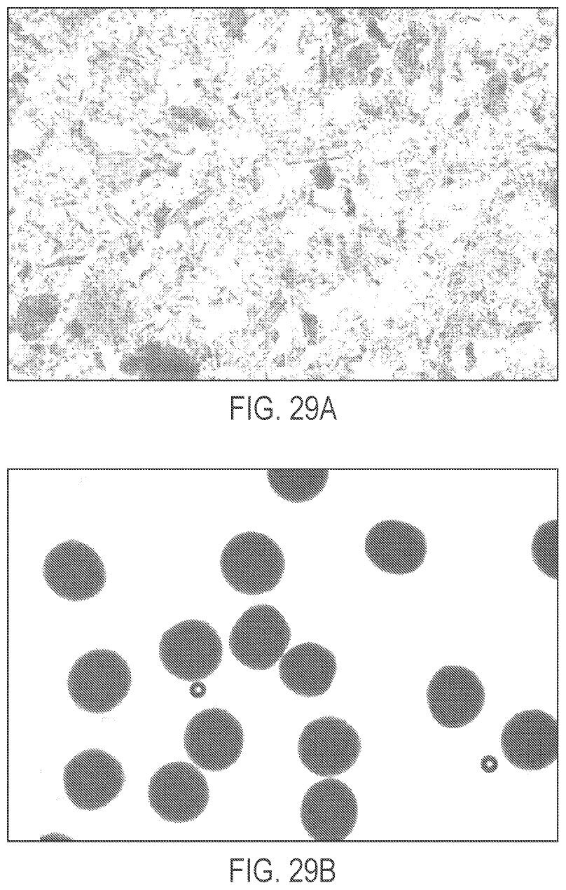

The following example describes a composition comprising a solid form of a polypeptide at least partially encapsulated by a hydrogel. Specifically, a composition comprising an amorphous monoclonal antibody solid encapsulated by alginate hydrogel particles was prepared. A precursor composition for generating amorphous mAb2 was developed and included a PEG 3350 concentration of 16 to 25 mg/ml to form the amorphous mAb2. FIG. 29A shows a microscopy image of the precursor composition comprising the amorphous mAb2. The amorphous mAb precursor composition was loaded inside sodium alginate hydrogel particles by combining with a 1% alginate VLVM (very low viscosity alginate high mannuronic acid rich) solution and using the centrifugal extrusion method described above at 400 RCF using a 30G nozzle with a 7 mm collection distance. FIG. 29B shows a microscopy image of the resulting amorphous mAb2-laden alginate hydrogel particle composition.

### Example 9

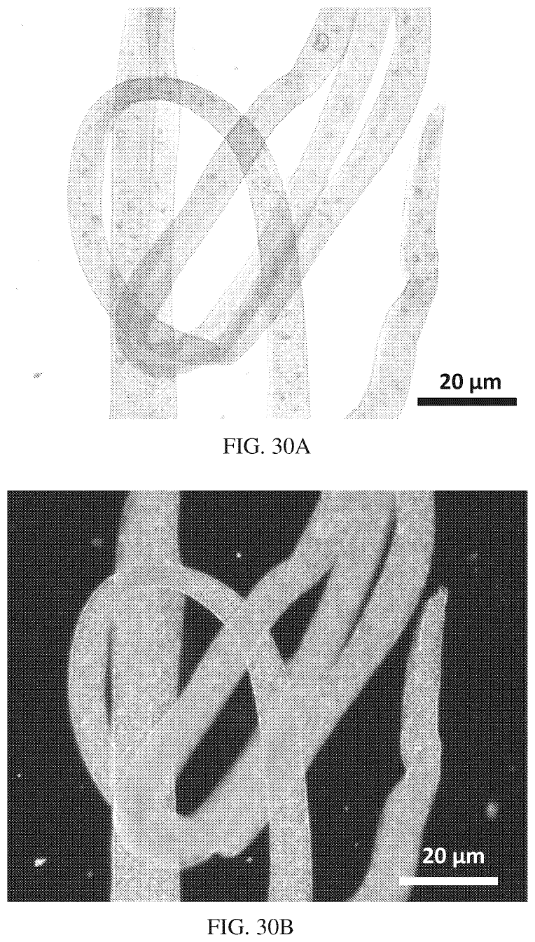

The following example describes a composition comprising a solid form of a polypeptide at least partially encapsulated by a hydrogel. A hydrogel comprising a natural polysaccharide, alginate, was formed through ionic crosslinking. The hydrogel cross-linked in the presence of the divalent cation Ca²⁺. The hydrogel particles were prepared via a centrifugal extrusion method as described above, but using 600 RCF and a tapered dispenser to provide higher alginate flow. FIGS. 30A-30B show bright field (FIG. 30A) and crossed-polarized (FIG. 30B) microscopy images of resulting mAb2 crystal-laden alginate hydrogel fiber particles.

### Example 10

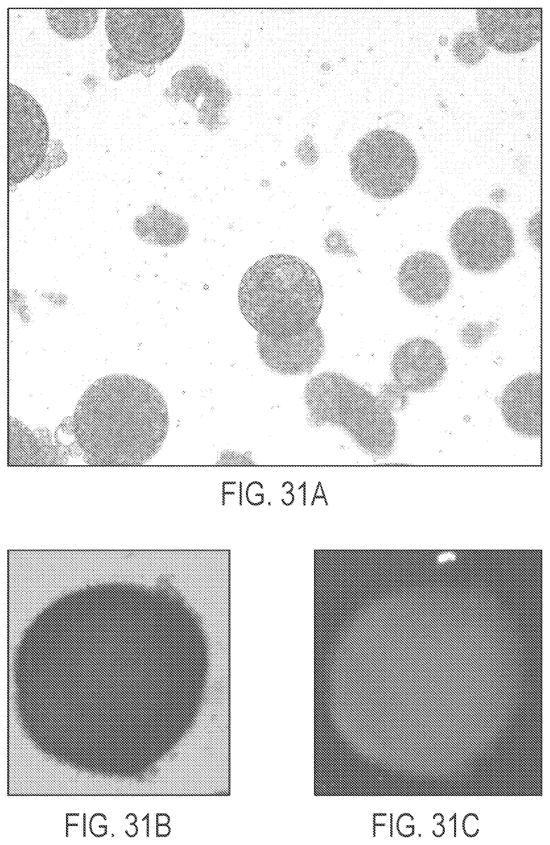

The following example describes a composition comprising a solid form of a polypeptide at least partially encapsulated by a hydrogel. A hydrogel comprising gelatin was formed through thermally gelation of particles. A prepolymer solution having a pH of 7.4 and containing 5% gelatin, 10% w/v PEG 3350, 50 mM HEPES, and mAb2 crystals was prepared at elevated temperatures (35-40° C.), and the hydrogel particles were later thermally formed at 4-20° C. The hydrogel particles were prepared via emulsion polymerization. FIGS. 31A-31C show microscopy images of mAb2 crystal-laden gelatin hydrogel particles formed via the thermal gelling and batch emulsification polymerization techniques.

### Example 11

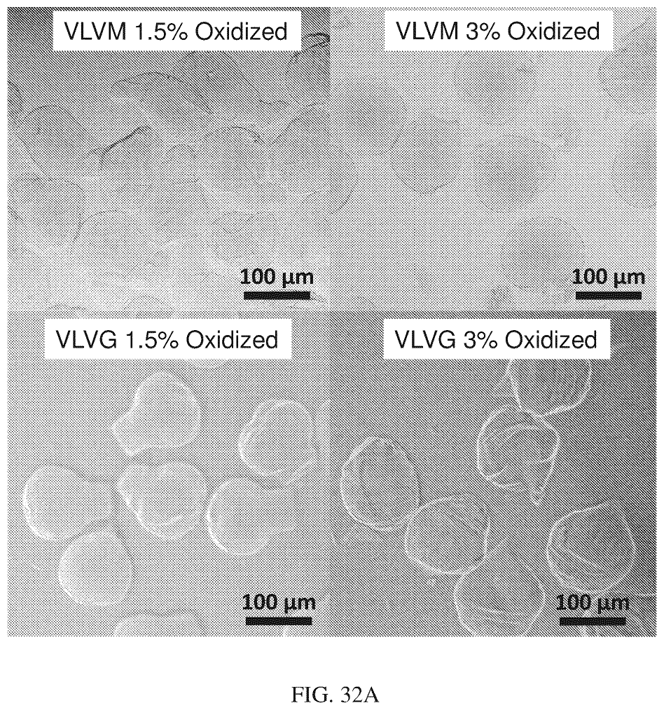

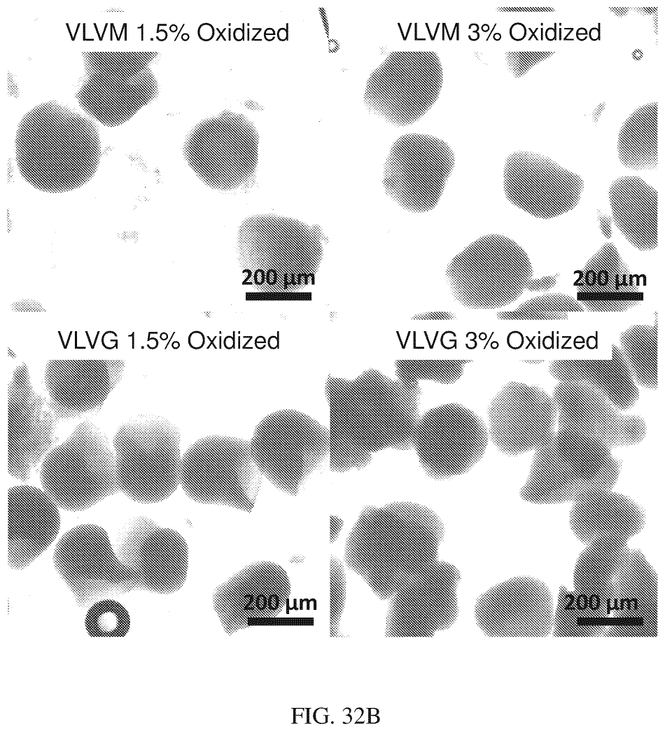

The following example describes a composition comprising a solid form of a polypeptide at least partially encapsulated by a hydrogel. A hydrogel comprising a chemically modified polysaccharide, partially oxidized alginate, was formed through ionic crosslinking. The hydrogel particles were formed of polysaccharide polymer chains that were partially oxidized, in this instance, to enhance their biodegradability properties. The partially oxidized alginate polymer chains were prepared by reacting sodium alginate with sodium periodate. The ratio of sodium periodate to uronic acid groups in the alginate was varied to generate 1.5% and 3% partially oxidized alginates. The hydrogel particles were formed using the techniques and conditions described in Example 3. FIG. 32A shows microscopy images of the hydrogel particles formed with the partially oxidized alginate. FIG. 32B shows microscopy images of mAb2 crystal-laden hydrogel particles formed with the partially oxidized alginate.

### Example 12

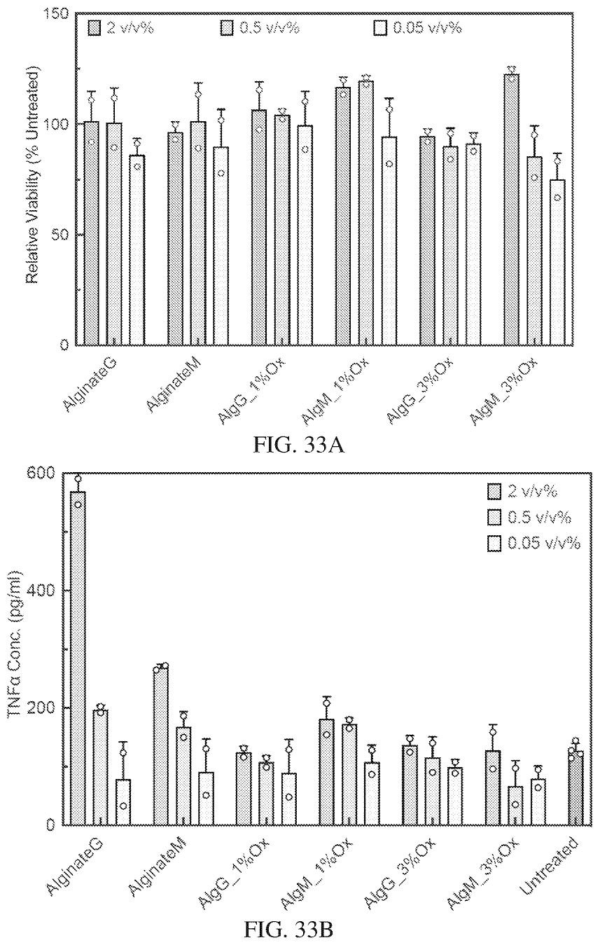

The following example describes testing of cell behavior upon exposure to hydrogel particles formed using the procedures described in this disclosure. FIG. 33A shows plots of cell viability of NIH Raw 264.7 cells in the presence of 0.05%, 0.5% and 2% v/v alginate hydrogel particles using alginates with varying viscosities and oxidation extents as indicated in the figure. Suitable viability was observed for each alginate hydrogel tested.

FIG. 33B shows plots of the amount of the cytokine TNF α (Tumor Necrosis Factor alpha) secretion from NIH Raw 264.7 cells in the presence of 0.05%, 0.5% and 2% v/v alginate hydrogel particles using alginates with varying viscosities and oxidation extents as indicated in the figure. It was observed that higher concentrations of hydrogel particles generally induced greater secretion of TNF α from the cells.

### Example 13

The following example describes testing of the quality of polypeptides released from hydrogel particles. Specifically, the functional stability and aggregation of antibodies released from hydrogel particles loaded with crystalline antibody was assessed.

To assess the functionality of mAb2 crystals subjected to hydrogel processing, a sample of mAb2 crystal-laden hydrogel particles was dissolved in PBS and analyzed by an enzyme-linked immunosorbent assay. The sample with mAb2 dissolved from the hydrogel particles indicated no loss in potency, demonstrating that the overall process (crystallization, encapsulation, dissolution, and subsequent handling) did not negatively affect the competitive binding functionality of mAb2 within the error of the assay.

Ultra-performance size exclusion chromatographic analysis of mAb2 samples was also performed to assess aggregation. A sample of untreated control mAb2 sample (i.e., free mAb2) was tested as a baseline measurement, and indicated 1.1% of sample eluting as an aggregate. A sample of mAb2 dissolved from 200 mg/mL PEGDA hydrogel microparticles that had been loaded with mAb2 crystals was tested and indicated 6.6% of sample eluting as an aggregate.

### Example 14

The following example describes testing of the quality of polypeptides within and released from hydrogel particles. Specifically, the amorphous nature and the functional stability amorphous solid-laden mAb2 alginate hydrogel particles were assessed using SONICC and enzyme-linked immunosorbent assay (ELISA) techniques.

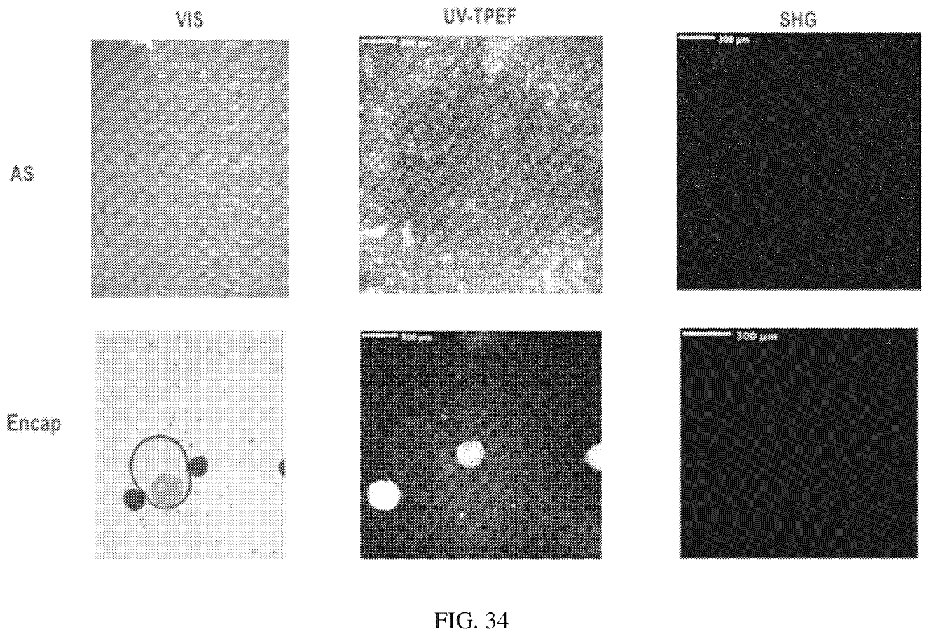

FIG. 34 shows bright field (VIS; left column), ultraviolet two-photon excited fluorescence (UV-TPEF; middle column), and second harmonic generation (SGH; right) microscopy images of a free suspension of amorphous mAb2 (top row, labeled as “AS” for amorphous suspension) and amorphous mAb2 solid-laden alginate hydrogel particles (bottom row, labeled as “Encap” for encapsulated). The results shown in FIG. 34 indicated that an amorphous solid form of mAb2 that was not crystalline in nature could be partially encapsulated within the hydrogel particles.

To assess the functionality of mAb2 subjected to hydrogel processing, a sample of amorphous solid mAb2-laden alginate hydrogel particles was dissolved in PBS and analyzed by an enzyme-linked immunosorbent assay. The sample with mAb2 dissolved from the hydrogel particles indicated no loss in potency, demonstrating that the overall process (preparation of solid form of polypeptide, encapsulation, dissolution, and subsequent handling) did not negatively affect the competitive binding functionality of mAb2 within the error of the assay.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B.” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of.” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of.” “only one of.” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States

Patent Office Manual of Patent Examining Procedures, Section 2111.03.

### SEQUENCE LISTING

The present specification is being filed with a computer readable form (CRF) copy of the Sequence Listing. The CRF entitled M092570816WO00-SEQ-TJO.txt, which was created on Jul. 30, 2021 and is 9,726 bytes in size, is incorporated herein by reference in its entirety.

1. A composition, comprising:
a hydrogel; and
a crystal comprising a solid form of a polypeptide at least partially encapsulated by the hydrogel.

2. A composition, comprising:
crystals comprising a solid form of a polypeptide present in an amount of greater than or equal to 1 mg/mL and less than or equal to 500 mg/mL,
wherein the composition has a dynamic viscosity that is at least 1.1 times lower than that of an aqueous suspension having an equivalent concentration of crystalline polypeptides under otherwise essentially identical conditions.

3. A composition, comprising:
crystals comprising a solid form of a polypeptide associated with one or more hydrogels such that less than or equal to 10 wt % of the crystals are aggregated.

4. (canceled)

5. The composition of claim 2, wherein the crystalline polypeptides are at least partially encapsulated by a carrier.

6. The composition of claim 5, wherein the carrier comprises a hydrogel.

7. (canceled)

8. The composition of claim 1, wherein the hydrogel comprises covalently cross-linked polymer chains, ionically cross-linked polymer chains, and/or thermally cross-linked polymer chains.

9. The composition of claim 8, wherein the cross-linked polymer chains are formed from polymer chains having a molecular weight of less than or equal to 75 kDa.

10. (canceled)

11. The composition of claim 1, wherein the hydrogel comprises a cross-linked polyalkylene oxide.

12. (canceled)

13. The composition of claim 1, wherein the hydrogel comprises a cross-linked polysaccharide.

14-16. (canceled)

17. The composition of claim 1, wherein the hydrogel comprises polypeptide chains.

18. (canceled)

19. The composition of claim 1, wherein at least some of the hydrogel is in the form of particles having the shape of sphere, spheroid, or fiber.

20. The composition of claim 19, wherein the particles have an average largest cross-sectional dimension of greater than or equal to 100 microns and less than or equal to 300 microns.

21. The composition of claim 19, wherein the particles have an average largest cross-sectional dimension of greater than or equal to 10 microns and less than or equal to 100 microns.

22. (canceled)

23. The composition of claim 19, wherein the particles have an average largest cross-sectional dimension of greater than or equal to 1 micron and less than or equal to 10 microns.

24. The composition of claim 19, wherein the particles have an average largest cross-sectional dimension of less than or equal to 1 micron.

25-31. (canceled)

32. The composition of claim 1, wherein the composition has a dynamic viscosity of less than or equal to 0.3 Pas at a temperature of 25° C. and under a shear rate of 100 s⁻¹.

33. The composition of claim 1, wherein the polypeptide can serve as a therapeutic polypeptide, as a prophylactic polypeptide, or as both a therapeutic polypeptide and as prophylactic polypeptide.

34. (canceled)

35. The composition of claim 1, wherein the polypeptide is an antibody.

36. The composition of claim 1, wherein the polypeptide is a monoclonal antibody.

37. The composition of claim 1, wherein the polypeptide is a monoclonal antibody of any subtype of IgG.

38-75. (canceled)


SEQUENCE LISTING (10 sequences)

SEQ ID NO: 1 - protein, 15 aa, Artificial Sequence
  Synthetic polypeptide
RASKGVSTSGYSYLH

SEQ ID NO: 2 - protein, 7 aa, Artificial Sequence
  Synthetic polypeptide
LASYLES

SEQ ID NO: 3 - protein, 9 aa, Artificial Sequence
  Synthetic polypeptide
QHSRDLPLT

SEQ ID NO: 4 - protein, 111 aa, Artificial Sequence [sequence omitted]

SEQ ID NO: 5 - protein, 218 aa, Artificial Sequence
  Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, Artificial Sequence
  Synthetic polypeptide
NYYMY

SEQ ID NO: 7 - protein, 17 aa, Artificial Sequence
  Synthetic polypeptide
GINPSNGGTNFNEKFKN

SEQ ID NO: 8 - protein, 11 aa, Artificial Sequence
  Synthetic polypeptide
RDYRFDMGFDY

SEQ ID NO: 9 - protein, 120 aa, Artificial Sequence [sequence omitted]

SEQ ID NO: 10 - protein, 447 aa, Artificial Sequence
  Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK